SEQUENCE LISTING (90 sequences)

SEQ ID NO: 1 - protein, 825 aa, Bifidobacterium adolescentis
MTSPVIGTPWKKLNAPVSEEAIEGVDKYWRAANYLSIGQIYLRSNPLMKEPFTREDVKHRLVGHWGTTPGLNFLIGHINRLIADHQQNTVIIMGPGHGGPAGTAQSYLDGTYTEYFPNITKDEAGLQKFFRQFSYPGGIPSHYAPETPGSIHEGGELGYALSHAYGAVMNNPSLFVPAIVGDGEAETGPLATGWQSNKLINPRTDGIVLPILHLNGYKIANPTILSRISDEELHEFFHGMGYEPYEFVAGFDNEDHLSIHRRFAELFETVFDEICDIKAAAQTDDMTRPFYPMIIFRTPKGWTCPKFIDGKKTEGSWRSHQVPLASARDTEAHFEVLKNWLESYKPEELFDENGAVKPEVTAFMPTGELRIGENPNANGGRIREELKLPKLEDYEVKEVAEYGHGWGQLEATRRLGVYTRDIIKNNPDSFRIFGPDETASNRLQAAYDVTNKQWDAGYLSAQVDEHMAVTGQVTEQLSEHQMEGFLEGYLLTGRHGIWSSYESFVHVIDSMLNQHAKWLEATVREIPWRKPISSMNLLVSSHVWRQDHNGFSHQDPGVTSVLLNKCFNNDHVIGIYFPVDSNMLLAVAEKCYKSTNKINAIIAGKQPAATWLTLDEARAELEKGAAEWKWASNVKSNDEAQIVLAATGDVPTQEIMAAADKLDAMGIKFKVVNVVDLVKLQSAKENNEALSDEEFAELFTEDKPVLFAYHSYARDVRGLIYDRPNHDNFNVHGYEEQGSTTTPYDMVRVNNIDRYELQAEALRMIDADKYADKINELEAFRQEAFQFAVDNGYDHPDYTDWVYSGVNTNKQGAISATAATAGDNE

SEQ ID NO: 2 - DNA, 2478 bp, synthetic construct
  source 1..2478: mol_type = other DNA
atgacctctccagttattggtactccatggaaaaaattgaacgccccagtttctgaagaagccattgaaggtgttgataagtattggagagctgccaactatttgtccattggtcaaatctacttgagatccaacccattgatgaaggaaccattcactagagaagatgtcaagcacagattggttggtcattggggtactactccaggtttgaattttttgatcggtcacatcaacagattgatcgccgatcatcaacaaaacaccgttattatcatgggtccaggtcatggtggtccagctggtactgctcaatcttatttggatggtacttacaccgaatacttcccaaacattactaaggatgaagccggtttacaaaagttcttcagacaattttcttacccaggtggtatcccatctcattatgctccagaaacaccaggttctattcatgaaggtggtgaattgggttatgctttgtctcatgcttatggtgctgttatgaacaacccatctttgtttgttccagctatagttggtgatggtgaagctgaaactggtccattggctactggttggcaatctaacaaattgattaacccaagaaccgacggtatcgttttgccaattttacatttgaacggttacaagatcgccaacccaacaattttgtccagaatctctgatgaagaattgcacgaattcttccatggtatgggttatgaaccatacgaatttgttgctggtttcgacaacgaagatcatttgtctatccatagaagattcgccgaattattcgaaaccgttttcgacgaaatttgcgatattaaggctgctgctcaaactgatgatatgactagaccattttacccaatgatcatcttcagaactccaaagggttggacttgtccaaagtttatcgatggtaaaaagaccgaaggttcttggagatctcatcaagttccattggcatctgctagagatactgaagctcatttcgaagttttgaagaactggttggaaagttacaagcctgaagaattatttgacgaaaatggtgccgtcaaacctgaagttactgcttttatgccaacaggtgaattgagaattggtgaaaatccaaatgctaacggtggtagaatcagagaagaattgaaattgccaaagttggaagattacgaagtcaaagaagttgccgaatacggtcatggttggggtcaattggaagctactagaagattgggtgtttacaccagagacatcatcaagaacaatccagactccttcagaatttttggtccagacgaaactgcttctaacagattgcaagctgcttacgatgttaccaacaaacaatgggatgctggttatttgtctgcccaagttgatgaacatatggctgttactggtcaagttaccgaacaattgtcagaacatcaaatggaaggttttttggaaggttacttgttgactggtagacatggtatttggtcctcttacgaatctttcgttcacgttatcgattccatgttgaatcaacacgctaaatggttggaagccactgttagagaaattccttggagaaagccaatctcctctatgaacttgttggtttcttcacacgtttggagacaagatcataacggtttttcacatcaagatccaggtgttacttctgtcttgttgaacaagtgtttcaacaacgatcatgtcatcggtatctacttcccagttgactctaatatgttgttggctgttgctgaaaagtgctacaaatctaccaacaagatcaacgctattatcgctggtaagcaaccagctgctacttggttgactttggatgaagctagagctgaattggaaaaaggtgctgctgaatggaaatgggcttctaatgttaagtctaacgacgaagctcaaatcgttttggctgctacaggtgatgttccaactcaagaaattatggctgcagctgataagttggatgctatgggtattaagttcaaggttgttaacgttgtcgacttggttaagttgcaatccgctaaagaaaacaacgaagccttgagtgacgaagaatttgctgaattattcaccgaagataagccagttttgttcgcctatcattcttacgctagagatgtcagaggtttgatatacgatagaccaaaccacgataacttcaacgttcatggttatgaagaacaaggttctactactaccccatacgatatggttagagttaacaacatcgacagatacgaattgcaagccgaagctttgagaatgattgatgcagataagtacgccgacaagattaacgaattagaagctttcagacaagaagccttccaattcgctgttgataatggttacgatcatccagattacaccgattgggtttattctggtgttaacactaacaagcaaggtgctatttctgctactgctgcaactgctggtgataatgaatga

SEQ ID NO: 3 - protein, 788 aa, Lactiplantibacillus pentosus
MSTDYSSPAYLQKVDKYWRAANYLSVGQLYLKDNPLLQRPLKASDVKVHPIGHWGTIAGQNFIYAHLNRVINKYGLKMFYVEGPGHGGQVMVSNSYLDGTYTDIYPEITQDVEGMQKLFKQFSFPGGVASHAAPETPGSIHEGGELGYSISHGVGAILDNPDEIAAVVVGDGESETGPLATSWQSTKFINPINDGAVLPILNLNGFKISNPTIFGRTSDEKIKQYFESMNWEPIFVEGDDPEKVHPALAKAMDEAVEKIKAIQKNARENDDATLPVWPMIVFRAPKGWTGPKSWDGDKIEGSFRAHQIPIPVDQTDMEHADALVDWLESYQPKELFNEDGSLKDDIKEIIPTGDARMAANPITNGGVDPKALNLPNFRDYAVDTSKHGANVKQDMIVWSDYLRDVIKKNPDNFRLFGPDETMSNRLYGVFETTNRQWMEDIHPDSDQYEAPAGRVLDAQLSEHQAEGWLEGYVLTGRHGLFASYEAFLRVVDSMLTQHFKWLRKANELDWRKKYPSLNIIAASTVFQQDHNGYTHQDPGALTHLAEKKPEYIREYLPADANSLLAVGDVIFRSQEKINYVVTSKHPRQQWFSIEEAKQLVDNGLGIIDWASTDQGSEPDIVFAAAGTEPTLETLAAIQLLHESFPEMKIRFVNVVDILKLRSPEKDPRGLSDAEFDHYFTKDKPVVFAFHGYEDMVRDIFFDRHNHNLHVHGYRENGDITTPFDVRVMNQMDRFDLAKSAIAAQPAMENTGAAFVQDMDNMLAKHNAYIRDAGTDLPEVNDWQWKGLK

SEQ ID NO: 4 - DNA, 2367 bp, Lactiplantibacillus pentosus
  source 1..2367: mol_type = other DNA
atgtctacagattactcatcaccagcatatttgcaaaaagttgataagtactggcgtgctgccaactatttatcagttggtcaactttatttaaaagataatcctttattacaacggccattaaaggctagtgacgttaaggttcacccaatcggtcactggggcacgattgccggccaaaacttcatctatgctcatcttaaccgggtcatcaacaagtacggtttgaagatgttctacgttgaaggtccaggtcatggtggccaagtgatggtctccaactcataccttgatgggacttacacggatatttatcctgaaattacgcaggatgttgaagggatgcaaaaacttttcaagcaattctcattcccaggtggcgtggcttcccatgctgctcctgaaacaccaggctcaatccacgaaggtggcgaacttggttactcaatttcacacggtgttggggcaatccttgacaaccctgatgaaatcgccgcagtcgttgttggtgacggggaatccgaaaccggtccattagcaacttcatggcaatcaacgaagttcatcaacccaatcaacgatggggcagtgttaccaatcttgaaccttaacggctttaagatttctaacccaacgatctttggtcggacttctgatgaaaagatcaagcaatacttcgaaagcatgaactgggaaccaatctttgttgaaggtgacgatcctgaaaaggttcacccagctttagctaaggctatggatgaagccgtcgaaaagatcaaagccattcaaaagaacgctcgtgaaaacgatgacgctactttaccagtatggccaatgatcgtcttccgcgcacctaagggctggactggtcctaagtcatgggatggcgacaagatcgaaggttcattccgagctcaccaaattccaattcctgttgaccaaaccgacatggaacatgccgatgcgttagttgactggttggaatcgtatcaaccaaaggaactcttcaatgaagatggttctttgaaggatgatatcaaagaaattatcccaactggcgatgcacggatggccgctaacccaatcactaatggtggggttgatccaaaggccttgaacttacctaacttccgtgattacgccgttgatacgtctaagcatggtgccaacgttaagcaagatatgatcgtttggtcagactacttgcgtgatgttatcaagaagaacccagataacttccggttatttggccctgatgaaaccatgtcaaaccggttatatggtgtctttgaaaccactaaccgtcaatggatggaagatattcacccagatagtgaccaatacgaagcacctgctggtcgggtcttggatgctcagttatctgaacaccaagctgaaggttggttagaaggttacgtcctaactggtcgtcatggcttgtttgcaagttacgaagccttcttacgggttgtcgactcaatgttgacgcaacacttcaagtggttacgtaaggccaacgaacttgactggcggaagaagtacccgtcactcaacattatcgcggcttcaactgtgttccaacaagaccataatgggtacacccaccaagatccaggtgctttgactcatttggctgaaaagaagcctgaatatatccgcgaatatttaccagccgacgccaactccttgttagctgttggggacgtcatcttccgtagccaagaaaagatcaactacgtggttacgtcgaagcacccacgtcaacaatggttcagcattgaagaagctaagcaattagttgacaacgggcttggtatcattgactgggcaagtacggaccaaggtagcgaaccagatatcgtgttcgctgctgccggaacggaaccaacgcttgaaacattggctgcaatccaattgctccacgaaagcttcccagaaatgaagattcgtttcgtgaatgtggtcgacatcttgaagttacgtagccctgaaaaggaccctcgtggcttgtcagacgctgaatttgaccattacttcactaaggacaaaccagttgtcttcgccttccatggttacgaagacatggttcgtgacatcttctttgatcgtcacaaccacaacttacacgttcatggctaccgtgaaaatggtgacattacgacaccattcgatgtccgggtcatgaaccaaatggaccgtttcgacttagcaaaatctgcaattgcggcgcaaccagcaatggaaaacaccggtgcagcctttgttcaagacatggataacatgcttgcaaaacacaacgcatacatccgtgacgccggaaccgacttgccagaagttaacgactggcaatggaaaggtttgaaataa

SEQ ID NO: 5 - protein, 796 aa, Lactiplantibacillus pentosus
MSEAIKSKSVDYSSDEYLKRVDEYWRAANYISVGQLYLLNNPLLQEPLKATDVKVHPIGHWGTIAGQNFIYAHLNRAINKYGLNMFYIEGPGHGGQVMVSNSYLDGTYTETYPKITQDKAGMKRLFKQFSFPGGVASHADPKTPGSIHEGGELGYSILHGAGAVLDNPGLIAATVVGDGESETGPLATSWQVNKFLNPITDGTVLPILNLNGFKISNPTVLSRESHEELEDYFKGLGWDPHFVEGTDPAKMHHLMAEELDKVIEEIHAIRQNAKDNNDESRPKWPMIVFRAPKGWTGPKSWDGEPIEGSFRAHQIPIPVDRNHMEHADKLVDWLKSYKPEELFDENGTLKPEIAAIIPEGQARMAANPVTNGGKLTKDLITPNIDDYALDNKDHGKEDGSDMTELGKYIRDLIKLNKDNKNFRGWGPDETLSNKLGAAFEDTKRQWMEPIHEPNDALLAPQGRIIDSMLSEHMDEGMLEAYNLTGRYGFFASYESFLRVVDSMLTQHFKWLRNSHEETPWRADVPSLNVIASSTAFQQDHNGYSHQDPGIISHLAEKKTEYVRAYLPGDANTLIATFDKAIQSKQLINLIIASKHPRPQWFTMDEAKHLVRDGLGVVDWASTDNGEEPDVVFATAGSEPTTESLAAVSILHARFPEMKIRFINVVDLLKLKKDDPRGLSDAEFDAFFTKDKPVIFAYHAYDDLVKTLFFDRHNHNLHVHGYREEGDITTPFDMRVRNELDRFHLVKAALLATPAYAEKGAHVIQEMNDILDKHHDYIRAEGTDIPEVENWKWTPLK

SEQ ID NO: 6 - DNA, 2391 bp, Lactiplantibacillus pentosus
  source 1..2391: mol_type = other DNA
atgagtgaagcaattaaatccaaatccgttgattactcttcagatgaatacttaaagcgtgttgatgaatactggcgcgcggcgaactacatctcagttggtcaactctatctgttgaacaatccactacttcaggaacctttaaaggcaactgatgttaaagtccacccaatcggccactggggtacgattgcgggtcaaaactttatctatgcccatttgaaccgggcaatcaataagtacgggctcaacatgttctacatcgaaggtcctggtcatggtggccaagtcatggtctcaaactcctacttagatgggacttacactgaaacctatcctaagatcacacaagataaagctgggatgaagcgcctattcaaacagttctcattcccaggtggggttgcttctcatgccgatcctaagacaccgggctcaatccacgaaggtggcgaacttggttactcaatcctgcatggtgctggtgcggtacttgataaccctggtttaattgcagcaacggttgttggtgacggcgaatccgaaactggcccattggcaacttcttggcaagttaacaagttcctcaacccgattactgacgggaccgtcttaccaatcttgaacttaaacggttttaagatttctaacccaacggtactttcacgtgaatcacatgaagaactagaagactacttcaaaggcctcggctgggatccacactttgttgaaggaacggatcctgctaagatgcatcacctcatggctgaagaattggataaagtcattgaagaaatccacgcaattcgtcagaatgccaaggataacaatgatgaatctcgtcctaagtggccaatgatcgtcttccgggcacctaaaggctggactggccctaagagctgggacggcgaaccaattgaaggttcattccgggcccaccaaattccaattcctgtcgatcgtaaccatatggaacacgccgacaagctggttgactggctcaagtcatacaaaccagaagaattatttgatgaaaacggcacgttaaaaccagaaattgcggcaatcattccggaaggccaagcacgaatggcagccaaccccgttaccaacggtggtaagttaactaaagatttaattacaccaaatattgatgattatgctttggataacaaggatcatggcaaggaagatggctcagatatgactgagcttggtaagtacatccgtgatctgattaaattaaataaggataacaagaacttccgtggttggggtcctgatgaaaccttgtctaacaaattaggcgctgcttttgaagatactaagcggcaatggatggaaccaatccatgagcctaacgatgctttgttagcccctcaaggccggatcattgattccatgttgtcagaacacatggacgaagggatgttggaagcttacaacttaactggtcgttacggtttctttgcaagttatgaatcattcttacgcgttgtggattcaatgttgacgcaacatttcaagtggttacggaattctcacgaagagactccatggcgggctgatgtgccttcattgaacgtgattgcctcatcaacggccttccagcaagatcataatggatactcccatcaagatccaggtatcatttcacatttggccgaaaagaagaccgaatatgtacgcgcttatcttccaggcgatgccaatacgttgattgcaacctttgacaaggcaattcaaagtaagcaattaatcaacttgattattgccagcaagcaccctcgtccacaatggttcacgatggacgaagctaaacacttagttcgcgatggtctgggtgtcgttgactgggcaagcactgataacggcgaagaaccagacgtggtctttgcaactgctggttccgaaccaacaaccgaaagcttagctgcggtttcaatcctgcatgcacgcttccctgaaatgaagattcgcttcattaacgttgttgatcttctgaagctgaagaaggatgatccgcggggcttatcagatgccgaatttgacgccttcttcactaaggataagccagtcatcttcgcataccatgcctatgatgacttagtaaagacactcttcttcgatcgtcataaccacaacttacatgttcatggttatcgtgaagaaggggacattaccaccccattcgacatgcgtgttcgtaatgaacttgaccgtttccacttagtcaaagctgcattattagcaacacctgcttatgccgaaaaaggtgcgcacgtcattcaagaaatgaacgacatcttggacaagcatcatgattatattcgcgctgaaggtactgatattcctgaagttgaaaactggaaatggacgccattgaaataa

SEQ ID NO: 7 - protein, 377 aa, Lactiplantibacillus pentosus
MSEDLMFVSPDKYIQGAGLLEHSASYINALGSHPLIMADALIWKIVGEDFATYLSQHDFDVTRVTFNGEASVQEIARITQIAKDNHNDIIIGLGGGKTLDTSKGIAEEVGAKLVIAPTAASADAPTAGLSVLYTEEGSFDQYKFYQRHSDLVLMDTQVIANAPVRTLISGIADAMATNIEAKATAQSHGTTMGNGAATFTGRAIAAECERVIWKYAYEAVESNVQHIVTPALDAIVEANTLLSGLGFENGGLAAAHAIHNGFTAVKGDVHHLTHGEKVAFGTMVELVLQGTSHEELNRYIEFYLTLGLPITLEATHLDQLSDEEMLQIGKMATMPDETMKNMPFKVTPREVVNAMKAASTFAHTYEATHTIKPILPA

SEQ ID NO: 8 - DNA, 2134 bp, Lactiplantibacillus pentosus
  source 1..2134: mol_type = other DNA
taacgcttacaagtacattatacaacttggatagtgattgtccagtttgaactgaaaatttttactttcttgcacctttcttaatatttgcgctagtcaaccagtcactgcctacagcgtaacaccaattgatgaattttctccaactaaacacatcgttttaggccaatttaaggtttacaaaattccagcattgcgcacaggccaaacgtgggtcaaaccggtctagcgcgcgacttcattccagtttattagcggcattcagcggctcacatcatggccacgcggacacattttcaattctgtccgttgcggattgtgtaagcgctaccataaactataagtgttaagtaacccaggccagcacgcgttgacactcatccccacttgggccagcgcaccgttttatcagccgccgctccatttccccgctgaccgtgttaccaaacagtgtcttaaattagttatcaaccaatccgattttgaggaggaaatttatcatgagtgaagatttaatgtttgttagccccgataagtatattcaaggtgccggactcttggaacattccgcaagctatatcaacgccctcgggagccatccgttgatcatggccgacgcgctgatttggaaaatcgtcggtgaagacttcgcgacttacttgagccaacacgattttgacgtcacacgggtcacctttaatggtgaagcctcagttcaagaaatcgcgcggatcacacagattgccaaggataaccataacgatattattatcggtctcggcggcggcaagactctcgatactagtaaagggatcgctgaagaagtgggtgccaagttagtcatcgcgccgactgcggcttcagcggatgcaccaaccgcgggcttatcagtgctctatactgaggaaggtagcttcgaccaatacaagttctaccaacggcacagcgacttagtcttaatggacacacaagtcatcgctaacgcgccagtccggacactcatctctgggattgctgacgcgatggccactaatattgaagcaaaggcgaccgcgcaatcccacggcacaacgatgggcaacggtgctgctacctttactggtcgggcaattgcggccgaatgtgaacgtgtcatttggaagtacgcctatgaagccgtggaatccaacgtccaacacattgtaacaccggcacttgacgccatcgtcgaagccaacaccctactctctgggctcggatttgaaaatggtggtttagcggctgcccacgccattcataatggcttcaccgccgttaagggcgacgtccatcatttgactcacggcgagaaagtggcctttggtaccatggttgaactcgtattacagggtacttcacacgaagaactcaatcgctacattgaattctacttgacccttggcttaccaatcacgctagaggcgacccatctcgatcaattatccgatgaagaaatgctgcaaattggtaagatggcgacaatgcctgacgaaaccatgaagaacatgcctttcaaagtcaccccacgcgaggtggtcaacgccatgaaggctgcatcgaccttcgcccacacttacgaagccacccatacgatcaaaccaatcttgccggcttagtaatcactagtagtcacagtcactgcttactttggaagtattgcggcttgatgtctgcaatgcacgtcggttctaataattaaaatggccgtttcagattcgagtctgaaacggccattttggcgtcgctgtaacgctattatcatgagtactggcaggcttaccttgattagtcggccttaaatccgcaacggacagtaccgccagtttaagctactagctatagagctactctgatcggtagcgcacgccaacgattagctgaattgatttttgaccaatcagtctagttggtatgcaatttcgctgcgattaccgaatcgtgccggtatattggaaggcatccgacgttgggtcctgcaaaaaggcttttaaatcgtccatattttcaacgacggccttgcctgacttcgccaacatgacgttgaatgtcccttctgccagggtttcgccatagtaagcaactgggatatccaagccatacgccgtacccgtctccc

SEQ ID NO: 9 - protein, 332 aa, Lactiplantibacillus pentosus
MKKIINDPKNVVEEMVDGLVRSYPQYLTKLPDTEAMVRSDQASMKGKVGLVSGGGSGHEPAHAGFVGQGMLSAAVAGQVFTSPTPDQIYAAIKAVDQGKGVFLVIKNYSGDVMNFDMAKDMAELDDIQVKSIVVDDDIAVKDSLYTQGRRGVAGTVLMHKILGAAADQGASLDELETLANQVMPQLKTIAVALSAATVPEVGKPGFELADDEIEFGVGIHSEPGYRREKIKPSKALVQELLGKLDDELKLQADHQYAVLVNGMGATPLMDQYVFCHDLLDELAARDIEPAFMKVGNYMTSIDMAGISLTLLDMTTTNWLDYLQYPVKTIAWS

SEQ ID NO: 10 - DNA, 999 bp, Lactiplantibacillus pentosus
  source 1..999: mol_type = other DNA
atgaagaaaattattaatgatccaaaaaacgtggttgaagagatggtcgatggtttggtacggtcctacccgcagtatttgacgaaattaccagatactgaagcgatggttcgtagcgaccaagcctcgatgaaaggtaaggtcggtcttgtaagtggtgggggcagtggtcatgaacccgcccacgctggttttgttggccaaggcatgttgagtgccgcggtcgccggtcaagtcttcacctcaccaacgccggatcagatttacgcggcgatcaaagccgttgaccagggtaaaggtgttttcctcgtcatcaaaaactattccggtgatgtaatgaatttcgacatggccaaggatatggctgagttggatgacattcaagtgaaatcgatcgtggtcgatgacgatattgcggtcaaggatagcttgtacacgcaaggtcggcgcggtgttgcgggaaccgttttgatgcataagattctcggtgccgcggcagatcaaggggcgagtctcgatgagcttgaaacgctagccaaccaggtcatgccacagctcaaaacgattgcggtcgcgttatccgctgctacggtcccagaagtcggcaagccaggctttgaattggcggacgatgagattgaatttggcgtcggcattcatagtgaacccggttaccgacgtgaaaaaatcaagccatccaaggcgttagtccaagaattgttaggtaagctagatgacgaactcaagttgcaagccgaccatcaatacgcggtgctcgttaatggaatgggcgcaacgccactcatggaccagtacgtgttctgccatgacttattggatgaattagctgcccgtgacatcgaaccggcgtttatgaaagtcggcaattacatgacgtcaatcgatatggcggggatttcactgacactgctagatatgacaaccactaactggcttgattacttacaatacccagttaaaacgattgcttggtcataa

SEQ ID NO: 11 - protein, 193 aa, Lactiplantibacillus pentosus
MLTVENTTAWLNQFAAQIEQHQSYLSELDTPIGDGDHGGNMARGLAAVKTALATQPADLTKLFQATAMALISKVGGASGPLYGTAFLEMAKQSKTDTDLGTLLAAALAGIEKRGGATTGDKTLIDVWAPVVAAVQAGKLTQTVIDDAVASTKPMVAKKGRASYLGERSAGHLDPGAVSSGYLFTTLLATEGIL

SEQ ID NO: 12 - DNA, 582 bp, Lactiplantibacillus pentosus
  source 1..582: mol_type = other DNA
atgttaacagttgaaaatacgacggcatggctgaaccaatttgccgctcagattgaacaacaccagtcttatttaagtgaattggacacccccatcggtgacggcgatcatggtggtaacatggcgcggggcttagcagccgtcaagacggctctagccacgcaacccgcagatttgacgaagctgtttcaagcgaccgccatggcgctgatcagcaaagtgggtggcgcttcaggccccttatacgggacggccttcttagagatggcaaaacagagtaagaccgatacggacctcggcaccttgctggccgccgcattagccggaattgaaaaacgcggtggtgcgacgactggtgacaaaaccttgattgacgtgtgggcgcccgtcgttgccgcggttcaagctggcaaattgacgcagaccgtgatcgacgatgcggttgccagcacgaagccaatggttgccaaaaaagggcgggcgtcttatttaggcgaacgttctgccggccatctggatccaggggccgtctctagtggttacttgtttacgaccttattagcaacggaggggattttatga

SEQ ID NO: 13 - protein, 122 aa, Lactiplantibacillus pentosus
MSLGIVVVSHVPEIAAGVQRLLAQVAKDVSITIAGGTDDDQVGTSMAKITEAFEANTADELLAFYDLGSAKMNLEMAIEMSTKPVHLYDTALVESAYTAASLLQADVPLADVEAQLAPLKIK

SEQ ID NO: 14 - DNA, 369 bp, Lactiplantibacillus pentosus
  source 1..369: mol_type = other DNA
atgagtctaggaattgttgttgtttcccacgtccctgaaatcgcagctggcgttcaacgactactcgcgcaagtggcgaaggatgtctccatcacgattgccggtgggacggatgatgatcaagtcgggacgagcatggcgaagatcacggaggcctttgaagccaatacggcggatgaactgttagcgttctatgaccttgggagtgccaaaatgaatctggaaatggcgattgaaatgagcaccaagccggttcacttgtatgacaccgccctagtcgagagtgcctacaccgcggcttcattgttgcaagcggacgtgccgttggcggatgtggaagcgcagttggcgccgttgaaaattaagtag

SEQ ID NO: 15 - protein, 568 aa, Zymomonas mobilis
MSYTVGTYLAERLVQIGLKHHFAVAGDYNLVLLDNLLLNKNMEQVYCCNELNCGFSAEGYARAKGAAAAVVTYSVGALSAFDAIGGAYAENLPVILISGAPNNNDHAAGHVLHHALGKTDYHYQLEMAKNITAAAEAIYTPEEAPAKIDHVIKTALREKKPVYLEIACNIASMPCAAPGPASALFNDEASDEASLNAAVEETLKFIANRDKVAVLVGSKLRAAGAEEAAVKFADALGGAVATMAAAKSFFPEENPHYIGTSWGEVSYPGVEKTMKEADAVIALAPVFNDYSTTGWTDIPDPKKLVLAEPRSVVVNGVRFPSVHLKDYLTRLAQKVSKKTGALDFFKSLNAGELKKAAPADPSAPLVNAEIARQVEALLTPNTTVIAETGDSWFNAQRMKLPNGARVEYEMQWGHIGWSVPAAFGYAVGAPERRNILMVGDGSFQLTAQEVAQMVRLKLPVIIFLINNYGYTIEVMIHDGPYNNIKNWDYAGLMEVFNGNGGYDSGAGKGLKAKTGGELAEAIKVALANTDGPTLIECFIGREDCTEELVKWGKRVAAANSRKPVNKLL

SEQ ID NO: 16 - DNA, 1707 bp, synthetic construct
  source 1..1707: mol_type = other DNA
atgagctatacggttgggacgtatttggccgaacggttagttcagattggcttgaagcaccattttgcagtagctggtgactataacttggtcttattggataacttgctcctgaacaaaaacatggaacaagtttactgctgtaacgaattgaactgtggcttcagtgctgaaggttacgctcgtgccaagggtgcagccgctgccgttgtcacgtactcggtgggtgcgttaagtgcctttgatgcaatcggtggcgcttacgcagaaaatttgccggtgattctcatcagtggtgccccaaataacaatgaccacgcggcaggccatgtattacatcacgcgcttggcaaaaccgattatcattatcagttagaaatggcaaaaaacattactgctgcggccgaagcaatctatacgccggaagaggcacctgcgaagattgaccacgtgatcaagacggctttacgggagaaaaagccagtctatctagaaatcgcatgtaatattgcttcaatgccgtgtgcggcaccgggaccagcgtctgccctcttcaatgacgaagcgagtgacgaagcgagtctgaatgccgcggtcgaggaaacgttaaaatttattgctaaccgtgataaagtcgctgtattggtggggtccaagttacgtgcagccggtgctgaagaggcggccgttaagtttgctgatgctttaggcggtgctgtcgcaaccatggctgccgcaaaaagttttttccctgaggaaaacccccattatatcggtacctcctggggtgaagtcagttacccaggtgtcgaaaagacgatgaaggaagccgatgcagtgattgccctggccccggtttttaacgattatagtacgactggctggaccgacattcccgatccaaaaaagctagtattggcggagccacgctcagtcgttgtcaatggtgttcggtttcccagtgtccacttaaaagattatctgacacgtctagcccaaaaggtttcaaagaaaacgggtgctcttgatttctttaagagtttaaacgcaggcgaattaaaaaaggccgctccggctgacccttctgcgcctttagtgaacgctgaaatcgcccgacaggttgaagcactcctgacaccaaatactaccgtgatcgccgaaaccggtgatagctggtttaacgcccaacgaatgaagttaccgaacggcgcgcgcgtcgaatatgaaatgcaatggggtcatatcggttggagtgttcccgctgcgtttggctatgccgtcggcgcacctgaacgtcgcaatattttaatggtgggcgatggctcttttcaattgacagctcaggaggtagcgcaaatggtccgtttgaagttgcctgttatcattttccttattaataactatggttatacaatcgaagttatgattcacgatggaccatacaacaatattaagaattgggattatgctggtttaatggaagtttttaatggcaacgggggctatgactctggagcgggaaaagggctaaaagcgaaaaccgggggcgagttagcggaggcaattaaggtggctctagccaacactgatgggcctactttaatcgaatgctttattgggcgcgaagattgtaccgaagagttagtgaaatggggaaagcgagttgctgcagcgaacagtcggaaaccggtgaacaaactattataa

SEQ ID NO: 17 - DNA, 1707 bp, synthetic construct
  source 1..1707: mol_type = other DNA
atgtcatataccgttggcacctatttggctgaacgtttggttcaaatcggcttgaagcaccacttcgctgttgctggcgattataacttggttttgttggataacttgttgttgaacaagaacatggaacaagtttattgctgcaacgaattgaactgcggcttctcagctgaaggctatgctcgtgctaagggcgctgctgctgctgttgttacctattcagttggcgctttgtcagctttcgatgctatcggcggcgcttatgctgaaaacttgccagttatcttgatctcaggcgctccaaacaacaacgatcacgctgctggccacgttttgcaccacgctttgggcaagaccgattatcactatcaattggaaatggctaagaacatcaccgctgctgctgaagctatctataccccagaagaagctccagctaagatcgatcacgttatcaagaccgctttgcgtgaaaagaagccagtttatttggaaatcgcttgcaacatcgcttcaatgccatgcgctgctccaggcccagcttcagctttgttcaacgatgaagcttcagatgaagcttcattgaacgctgctgttgaagaaaccttgaagttcatcgctaaccgtgataaggttgctgttttggttggctcaaagttgcgtgctgctggcgctgaagaagctgctgttaagttcgctgatgctttgggcggcgctgttgctaccatggctgctgctaagtcattcttcccagaagaaaacccacactatatcggcacctcatggggcgaagtttcatatccaggcgttgaaaagaccatgaaggaagctgatgctgttatcgctttggctccagttttcaacgattattcaaccaccggctggaccgatatcccagatccaaagaagttggttttggctgaaccacgttcagttgttgttaacggcgttcgtttcccatcagttcacttgaaggattatttgacccgtttggctcaaaaggtttcaaagaagaccggcgctttggatttcttcaagtcattgaacgctggcgaattgaagaaggctgctccagctgatccatcagctccattggttaacgctgaaatcgctcgtcaagttgaagctttgttgaccccaaacaccaccgttatcgctgaaaccggcgattcatggttcaacgctcaacgtatgaagttgccaaacggcgctcgtgttgaatatgaaatgcaatggggccacatcggctggtcagttccagctgctttcggctatgctgttggcgctccagaacgtcgtaacatcttgatggttggcgatggctcattccaattgaccgctcaagaagttgctcaaatggttcgtttgaagttgccagttatcatcttcttgatcaacaactatggctataccatcgaagttatgatccacgatggcccatataacaacatcaagaactgggattatgctggcttgatggaagttttcaacggcaacggcggctatgattcaggcgctggcaagggcttgaaggctaagaccggcggcgaattggctgaagctatcaaggttgctttggctaacaccgatggcccaaccttgatcgaatgcttcatcggccgtgaagattgcaccgaagaattggttaagtggggcaagcgtgttgctgctgctaactcacgtaagccagttaacaagttgttgtag

SEQ ID NO: 18 - protein, 383 aa, Zymomonas mobilis
MASSTFYIPFVNEMGEGSLEKAIKDLNGSGFKNALIVSDAFMNKSGVVKQVADLLKAQGINSAVYDGVMPNPTVTAVLEGLKILKDNNSDFVISLGGGSPHDCAKAIALVATNGGEVKDYEGIDKSKKPALPLMSINTTAGTASEMTRFCIITDEVRHVKMAIVDRHVTPMVSVNDPLLMVGMPKGLTAATGMDALTHAFEAYSSTAATPITDACALKAASMIAKNLKTACDNGKDMPAREAMAYAQFLAGMAFNNASLGYVHAMAHQLGGYYNLPHGVCNAVLLPHVLAYNASVVAGRLKDVGVAMGLDIANLGDKEGAEATIQAVRDLAASIGIPANLTELGAKKEDVPLLADHALKDACALTNPRQGDQKEVEELFLSAF

SEQ ID NO: 19 - DNA, 1152 bp, synthetic construct
  source 1..1152: mol_type = other DNA
atggccagctccacgttttacattccatttgttaacgaaatgggagaaggttcattagaaaaggccattaaagatctgaacgggagtggattcaagaacgcgctgattgtgtcagatgcatttatgaataagagtggcgtggtaaagcaagtcgcggatttgttaaaggcacaaggcatcaactctgccgtctacgatggggtcatgccgaacccgactgtcacggcggttctagaagggttgaagattctgaaggacaataactcggattttgtaattagtctaggaggcggtagtccgcacgactgcgccaaggctatcgcattggttgctacgaacggtggcgaagtaaaggattacgaggggattgataaatcgaagaaacccgcactgccgttgatgagcattaatacgacagcggggacggctagtgaaatgacacgtttttgtatcattacggacgaagtccgccacgttaagatggcaatcgtcgatcggcacgttacgccgatggtctccgtcaacgatcctctactcatggttggaatgccaaaaggattaactgccgctaccggcatggacgccttgacgcatgcgtttgaagcttattcgagcacggcagcgactccaattaccgacgcatgcgccttaaaggctgcctcaatgatcgctaaaaatttaaaaacggcgtgcgataacggcaaggacatgcccgcgcgtgaagcaatggcctacgcgcaattcctggcgggcatggcgttcaataacgctagtttgggctacgtccacgcgatggcgcatcaattgggagggtactataacttgccacatggtgtgtgtaatgccgttttattgccccatgttctcgcctataacgcgtctgttgtcgcaggccggttaaaagatgttggcgtggcgatgggtttagacatcgcgaatcttggggacaaagagggtgccgaagcaacgatccaagcggtgcgggacctcgccgcaagtattggcattcctgcgaacctcacagaactcggagctaaaaaggaagacgttcccctgttagcggaccatgccctgaaagacgcatgtgccttaacaaacccgcgtcagggtgaccagaaggaagtcgaggaattattcttgagcgcgttttga

SEQ ID NO: 20 - DNA, 1151 bp, synthetic construct
  source 1..1151: mol_type = other DNA
atggcttcatcaaccttctatatcccattcgttaacgaaatgggcgaaggctcattggaaaaggctatcaaggatttgaacggctcaggcttcaagaacgctttgatcgtttcagatgctttcatgaacaagtcaggcgttgttaagcaagttgctgatttgttgaaggctcaaggcatcaactcagctgtttatgatggcgttatgccaaacccaaccgttaccgctgttttggaaggcttgaagatcttgaaggataacaactcagatttcgttatctcattgggcggcggctcaccacacgattgcgctaaggctatcgctttggttgctaccaacggcggcgaagttaaggattatgaaggcatcgataagtcaaagaagccagctttgccattgatgtcaatcaacaccaccgctggcaccgcttcagaaatgacccgtttctgcatcatcaccgatgaagttcgtcacgttaagatggctatcgttgatcgtcacgttaccccaatggtttcagttaacgatccattgttgatggttggcatgccaaagggcttgaccgctgctaccggcatggatgctttgacccacgctttcgaagcttattcatcaaccgctgctaccccaatcaccgatgcttgcgctttgaaggctgcttcaatgatcgctaagaacttgaagaccgcttgcgataacggcaaggatatgccagctcgtgaagctatggcttatgctcaattcttggctggcatggctttcaacaacgcttcattgggctatgttcacgctatggctcaccaattgggcggctattataacttgccacacggcgtttgcaacgctgttttgttgccacacgttttggcttataacgcttcagttgttgctggccgtttgaaggatgttggcgttgctatgggcttggatatcgctaacttgggcgataaggaaggcgctgaagctaccatccaagctgttcgtgatttggctgcttcaatcggcatcccagctaacttgaccgaattgggcgctaagaaggaagatgttccattgttggctgatcacgctttgaaggatgcttgcgctttgaccaacccacgtcaaggcgatcaaaaggaagttgaagaattgttcttgtcagctttcta

SEQ ID NO: 21 - protein, 347 aa, Enterococcus sp.
MEKVFVSPMTYRQGENVLWNHLEDILAFGQKGLIVTDGFVYQMLGERLYQKLTKLGGEMFVWVIEEDRPSETFDYIVALGGGRAIDLGKALAFESNASCIVVPTSAATDAPTSRISVKYHNGYFEKYSYYAKSPDLVLIDTAILINSPVHFLTAGIADGLSTSIEARTVRENSGVNTLGAAPTLAAEAIAEKCREVLLEFAEEAVEANEKHIITPAFEAVVEANTLLSGIGFESGGLSIAHALHNAMVSLWGNEIKGSHGQIIALTTLQQLRIEGREEELARYQTLFEKLALPNFYEELSIFPAAEELKKVTELAFSPEDGILRSHSPATKTNVYKNLLTFRKTREI

SEQ ID NO: 22 - protein, 336 aa, Enterococcus sp.
MPERKDGNKLKKIMNDPSNIVEEMLEGLVKSYPELVHRVEFSRVVAKNQKAEQVGLVSGGGSGHEPSHAGFVGEGMLSAAVLGDVFTSPTPDQIQTAIKEADSGKGVLLIVKNYTGDALNFDMAKELAAMDDIEVESVIVDDDIAVENSTYTAGKRGVAGTVLVHKIVGDAARNGASLAELKELGEKVVQATKTIGVALRAATVPEVGKPGFELGEDEIEYGVGIHGEPGYRREKMQPSKVLAKELVTKILDDYSNLPKEAGVFVNGMGGTPLMEQFVFMNDVLALLEERGINVVFRKVGNFMTSLDMQGLSLTLIDLTETQWKDSLESNVQTISW

SEQ ID NO: 23 - protein, 201 aa, Enterococcus sp.
MKLTVQEIQEWLSQFAEAINQNKQHLSDLDTPIGDGDHGNNMGRGVSAYEEAFQTDHPETISDTFKVFSMAMISKVGGASGPLYGSAFMNMMKATKEVDSIDSQEKLGEVIEQGTAGIQSRGKAEADDKTMLDVWLPVTEALKGGNLTKEIIDQAKEHTKDLVAKKGRASYLGERAIGHIDPGAASSAILFTTLLNVIKTS

SEQ ID NO: 24 - protein, 132 aa, Enterococcus sp.
MKADILLVSHSKMITDGIKEMIEQMNASEEITIHSLGGTSDGSLGSDPMKIIDTINEADSDREFLIFADLGSAVLSSELAFDMLEEDQQKHYHLVDAPLVEGAFASAITAGVSDDLTQILAEAQNAGKKGWN

SEQ ID NO: 25 - protein, 395 aa, Lactiplantibacillus pentosus
MQKTLIINAGSSSLKWQLFAMPAETVVASGLVERISMPGSIFTIKYGDHQKFEKTVDQLSQSQAAQMLLDELQQLAIIESLSEITAVAHRVVAGGETFKQAVAITPEVLTAIKQLSDFAPLHNPMEARGIETMAKALPDVTQYAVFDSQFFTDLPEMNAIYSLPYDLTQQYHIRRYGEHGISHGYLTGRAATLLDQPQSEVNLVTLHLGSGSSLAAVKAGRAFDTSMGFTPLTGVTMGTRAGDVDPALLPFLMKVLKTTDPNEIMTLLNDQSGLLGLSGVSSDMREIRAQEATNPQAHLAIEIFINRIVKYAGSYLTELKHVDALVFAGGIGEHNPKLRQQIVDELSIFGVKLDVDLNAAGQEGLISSPDSQIKVLLIPTNEELAMVRQVAALQN

SEQ ID NO: 26 - DNA, 1188 bp, Lactiplantibacillus pentosus
  source 1..1188: mol_type = other DNA
atgcagaaaacattgattattaatgcgggtagttcgtctttgaaatggcaattatttgccatgcctgccgaaaccgtagtcgcgagtgggttggttgaacgaatcagtatgcctggttcgatttttaccatcaagtacggtgaccatcagaaatttgagaagactgtcgaccaattgagtcaaagtcaggctgctcaaatgctattagatgaattacagcagttagcaattattgaatccttatcagaaatcacggcggtggcgcaccgcgtcgtggcgggtggcgagaccttcaagcaggcggtcgcaatcacgccggaagtgttgaccgccatcaaacagttgagtgattttgcaccattgcacaatccgatggaagcgcgtggtatcgaaacgatggccaaagccttgccagacgtgacgcaatacgccgtgtttgatagtcagttctttaccgatttgcctgaaatgaacgcgatttatagtctgccctatgatttaacgcagcaatatcacattcgccgctatggtgaacacggcatttcccacggttacttgaccgggcgtgcggccacgctattagatcagccacaatcagaagttaacctggtgacgttgcatttagggagcggttcttccctagctgcagtcaaagctggccgggcgtttgatacgtcgatgggctttacaccgctaactggggtgacgatgggcacgcgtgcgggggatgtcgacccggcattgctgccatttttgatgaaggtcctaaagacgactgacccgaacgaaatcatgacgttactcaatgaccagtccggcttattaggactctcaggagtgtcctcagatatgcgtgaaattcgagcccaggaagccaccaatccgcaggcccacttggcgattgaaatcttcatcaaccgaatcgtcaagtacgctgggagttatctgacagagttgaaacacgtggatgcgttagtcttcgctggcggtatcggtgagcataatcccaagctccggcaacaaatcgttgatgaactcagcatttttggtgtcaaactggatgtggacttgaacgctgccgggcaggaaggcctcatcagcagtcctgattcgcaaatcaaagtgttgctgattccaactaacgaagagttggcaatggtgcgccaggtcgcagcgctgcaaaattaa

SEQ ID NO: 27 - protein, 398 aa, Lactiplantibacillus pentosus
MAKILAINAGSSTLKWKLFSVPEETVIASGMVDRLGLPNSVFTIKKADGSKETETKDTIDAKEAAAMVLTRLKNDNIVDQLTDIKGVGHRVVAGGEDFKDSVVITPQTLKKIKDLSEYAPLHNPTQAYYIEVFRDLLPKAVQVAVFDTSLYADMPKVNYLYSIPYDYYEKFGARKYGAHGTSHRYVANRTAQLLGKPLDELKLVTLHLGSGASITAFDHGQAIDTSMGFTPLAGITMSTRSGDIDASIIPFLMRHLGISDVEDFIDILNHKSGLLGISGLSPDMRDLDKTQDTREQSKLAIEIFVNRVVKYVGSYIAEMNGIDALVFTAGMGENSWMIRERIAKSLGYFGITVDVDKNHMHGKEEVISKEGEPVKVMVVPTNEELMIVRDVERLSSAK

SEQ ID NO: 28 - DNA, 1197 bp, Lactiplantibacillus pentosus
  source 1..1197: mol_type = other DNA
atggcaaaaattttagcaattaacgccggcagttcgactttgaaatggaaacttttcagtgttccagaagaaacggttatcgcatcaggaatggttgaccggctgggtttaccgaactctgtttttacgattaaaaaagcagatggaagtaaagagaccgaaaccaaggatacgattgacgccaaagaagctgccgcaatggttttaacgcggttgaagaatgataatatcgtcgaccaactcaccgatatcaaaggggttggacaccgggtcgtcgctggtggcgaagacttcaaggactctgttgtgattacgccgcaaacattgaagaaaatcaaggatttatctgagtatgcgccgttacataacccaacgcaggcttactatatcgaagtgttccgtgatctgttaccaaaagcggttcaagtcgcggtctttgatacatcgctgtatgcggatatgccaaaagttaattatttatacagcattccatacgactactatgaaaaattcggtgcacggaagtatggtgctcacgggactagtcaccgttacgtggccaaccggaccgctcaattactgggcaagcccctcgatgaattgaagttagtcaccctgcacttgggttctggcgcttcgatcacggcctttgatcatggacaagctattgatacgtcaatgggcttcacgccattagccgggattacgatgagtacgcgttctggtgatatcgatgcgtcgattattccgttcttaatgcggcacctcggtatttcagacgttgaggacttcatcgatattttgaaccacaagtccggcctcttgggtatttcaggcttgtcaccagacatgcgtgacctggataagacgcaagacacccgtgaacagtcgaagcttgcgattgaaatcttcgtgaaccgggttgtgaagtacgttgggagttacattgccgaaatgaacggtattgatgcactcgtcttcaccgccggcatgggtgaaaactcatggatgattcgggaacgtatcgccaagtcactgggttacttcggtatcacggtcgacgttgacaagaatcacatgcacggcaaggaagaagtcatcagtaaggaaggcgaaccagttaaagtaatggtggtgccaaccaacgaagaattgatgattgttcgggatgttgaacgcttatcttcagctaaataa

SEQ ID NO: 29 - protein, 325 aa, Lactiplantibacillus pentosus
MDLFESLSQKITGQDQTIVFPEGTEPRIVGAAARLAADGLVKPIVLGATDKVQAVAKDLNADLAGVQVLDPATYPAEDKQAMLDSLVERRKGKNTPEQAAKMLEDENYFGTMLVYMGKADGMVSGAVHPTGDTVRPALQIIKTKPGSHRISGAFIMQKGEERYVFADCAINIDPDADTLAEIATQSAATAKVFDIDPKVAMLSFSTKGSAKGDMVTKVQEATAKAQAAAPELAIDGEMQFDAAFVEKVGLQKAPGSKVAGHANVFVFPELQSGNIGYKIAQRFGHFEAVGPVLQGLNKPVSDLSRGCSEEDVYKVAIITAAQGLA

SEQ ID NO: 30 - DNA, 978 bp, Lactiplantibacillus pentosus
  source 1..978: mol_type = other DNA
atggatttattcgagtcattatcacaaaagattactggtcaagatcaaacaattgtttttcctgaaggcactgaaccccgaattgtcggcgccgctgcacggttagcagcggatggcttagttaagccgatcgtgttaggcgcaacggacaaggttcaggccgtggctaaagacttgaacgctgatttagcaggtgttcaagtccttgatcctgcgacatacccggctgaagataaacaagcaatgcttgattcactggttgaacggcgtaagggtaagaacacgccagaacaagctgctaagatgttggaagatgaaaactactttggcacgatgctcgtatacatgggcaaggctgacgggatggtttctggtgccgtgcacccaactggtgatacggtgcgtccagccttacaaattatcaagaccaagccaggttcacaccgcatttctggggccttcatcatgcaaaagggtgaagaacggtacgtcttcgcggactgtgccatcaacattgatcccgatgcagatacgttagccgaaattgcgacccagagtgcggcgactgccaaggtctttgatattgacccgaaagtcgcaatgctgagcttctcgactaaggggtctgccaagggcgacatggtcactaaagttcaggaagcaacggccaaggcacaagcggctgcacctgaattagcgattgatggtgaaatgcaatttgacgccgcctttgttgaaaaggttgggctgcaaaaggcgccaggttcaaaagttgcgggtcacgccaatgtcttcgtcttcccagaactccaatctggtaacattggctacaagattgcgcaacgtttcggtcatttcgaagccgttggtcccgttctgcaaggcttgaacaagcccgtttctgacttgtcacgtggttgcagcgaagaagatgtctacaaggtcgccatcattaccgcggctcaaggcttagcttaa

SEQ ID NO: 31 - protein, 867 aa, Lactiplantibacillus pentosus
MIKTEKNQTTKVTDEVDQLVQRSKKALAVLKSYTQEQIDDLCEKVAVAALDNHMKLAKLAVEETGRGVVEDKAIKNIYASEYIWNSMRHDKTVGVIKEDDEEQLMEIAEPVGIVAGVTPVTNPTSTTVFKTLISLKGRNTIVFGFHPQAQKCSSAAADVMREAIKAAGGPADAVLYIEHPSIEATDALMHHTDVATILATGGPSMVTAAYSSGKPALGVGPGNGPTYVEKTADIKQAVNDIVLSKTFDNGMICASENSAIIDKEIYAEVKAEFIRLGCYYVKPKDVQALSDAVIDPNRHTVRGPVAGKTAYEIAQMAGLKDVAKDCRVLIAEINGVGVKYPLSGEKLSPVLTVYKADSHEAAFKRADELLHYGGLGHTAGIHTTDDDLVKEFGLQMPACRILVNTPSSVGGLGNIYNNMEPSLTLGTGSYGGNSISHNVTDMDLINIKTVAKRRNNMQWVKMPPKVYFERNSVRYLEHMAGIKKVFLVCDPGMVEFGYADRVTAVLNKRTDPVDIDIFSEVEPNPSTDTVYKGVARMKAFKPDTIIALGGGSAMDAAKGMWLFYEHPEASFLGAKQKFLDIRKRTYKVPVSEKVTYIGIPTTSGTGSEVTPYAVITDSKTHVKYPITDYAMQPDIAIVDPQFVETVPKRTTAWTGLDVITHATEAYVSTMASDYTRGWSIEALQLAFKYLKASYDGDKKAREKMHNASTLAGMAFANAFLGINHSIAHKLGGEFNLPHGLAIAITYPQVVRYNAEIPTKLAMWPKYNHNTALADYANIARALGLPGKTDEELKESLVKAYIDLAHSMDVTLSLKANRVEKKHFDATVDELAELAYEDQCTTANPREPLISELKAIIEREWDGQGTEK

SEQ ID NO: 32 - DNA, 2604 bp, Lactiplantibacillus pentosus
  source 1..2604: mol_type = other DNA
atgattaagacagaaaagaaccaaaccaccaaggttaccgacgaagtagaccagttagttcaacgttcaaagaaagcattggccgttttaaaatcgtatacacaagaacaaattgatgatctatgtgaaaaagttgcagtcgctgcacttgataatcatatgaaactcgccaagttggccgttgaagaaactggccgtggggtggttgaagataaagccatcaagaacatctacgccagtgaatatatttggaacagcatgcgccacgacaagaccgttggcgtcatcaaagaagacgatgaagaacaactaatggaaatcgctgaaccagtcgggatcgtggccggggtcacccccgttacgaacccaacttcaacgacagtcttcaagactttgatctccttgaagggtcgtaacacgatcgtctttggtttccatccacaagctcagaagtgtagttccgcggccgctgatgtgatgcgtgaagccatcaaagctgccggtggtcctgccgatgccgttttatacatcgaacatccaagtattgaagcgacggatgcgttgatgcaccataccgacgtagcgacgatcttagccactggtggtccaagcatggttaccgcagcctactcatcaggcaaaccagccttaggtgttggccctggtaacggcccgacctacgttgaaaagacggctgacatcaagcaagccgttaacgacattgtcttatccaagacgtttgataacgggatgatctgtgcctctgaaaacagtgccattattgataaagaaatttatgcggaagtcaaagcagaatttattcgtctcggttgctactatgtaaaaccaaaagacgttcaagctttaagtgatgccgttatcgatcctaaccggcacaccgttcggggaccagttgctgggaagactgcttacgaaatcgcccagatggctggattaaaggatgttgccaaggattgccgcgtcttgattgctgaaatcaacggtgtcggggtcaagtatccattatctggcgaaaaattatcaccagttttgaccgtttacaaggctgattcacatgaagctgcgttcaaacgggccgatgaattacttcattatggtggtttaggccatactgctgggattcacacgactgatgatgatttggtcaaggaattcggccttcaaatgccagcctgccggatcttagtgaacacgccatcttcagtaggtggtttagggaacatctacaacaatatggaaccttcattaacactgggcacggggtcatatggtggcaactcgatttctcataacgttacggatatggatctgattaatatcaaaactgtggcaaaacggcgtaataacatgcaatgggtcaaaatgcctcccaaagtctacttcgaacgcaattccgttcgctacttggaacacatggctgggattaaaaaggttttcctagtttgtgaccctgggatggtggaatttggctacgctgaccgtgtaacggctgtcttgaacaagcggacggatccggtcgatatcgatatcttctctgaagttgaaccgaacccatcgacggatacggtttacaaaggggtcgcacggatgaaggccttcaagccagatacgatcatcgcccttggtggtggttccgcaatggacgcggccaaaggaatgtggctcttctatgaacatccagaagcttccttcttaggtgctaagcagaagttcttggatattcggaaacggacctacaaagttcccgtatctgaaaaggtcacctatattgggattccaacgacttctgggacgggttctgaagtgacgccatacgccgttattacggactccaagacgcacgttaagtacccaatcaccgactacgcaatgcaaccagacattgccatcgttgacccacaatttgttgaaacggtgccaaagcggacgactgcttggactggcctggatgtcatcacccatgctactgaagcgtacgtttcaacgatggcctctgactacacgcgtggctggtcaattgaagccttacaattagccttcaagtacttgaaagcttcttatgatggcgacaagaaggcccgtgagaagatgcacaatgcgtcaaccttagccgggatggcctttgccaacgccttcttaggaattaaccactccatcgcgcataaattaggtggggaattcaacttaccacacgggttagcgattgccatcacttacccacaagttgtgcgctacaatgctgagattccaaccaagttagcaatgtggccgaagtacaaccacaacactgccttagccgactacgccaacattgcccgggccttagggttaccaggtaagacggacgaagaactcaaggaaagcttagttaaagcctatatcgacttggctcactcaatggatgtcaccttatccttgaaagccaaccgggttgaaaagaagcactttgacgccaccgtcgacgaattagctgaattggcctacgaagaccaatgcacgaccgcaaaccctcgcgaacctttaatcagtgaattaaaggcgatcatcgaacgcgaatgggacggccaaggtactgaaaaatag

SEQ ID NO: 33 - protein, 456 aa, Lactiplantibacillus pentosus
MLKEIDEHTTVSQSIDRLVLNASLAATRLEVMNQEQVDQAVAAMARAAHAARGMLAAMAVEETGRGNYRDKVAKNDFAAKHIYDYIKDDKTVGIINDDPVSGVMKVAEPVGIIAGVTPVTNPTSTVIFNAMLALKTRNPIIFGFHPFAQKSCVETGKIIRDAAVTAGAPKDWIQWIKTPSLEATNTLMNHPGVATIIATGGAGMVKTAYSTGKPALGVGPGNVPCFIEQTADVHQAVSDVVTSKSFDNGMICASESNLIVADQIYDQVKTELSQQGVYFVGPDNFKALEATVMNLDKQAVDPKVAGQTPWQIAQWAGFDVPTDTKVLAVELPSIGGDQVLSGEKLSPVLAVVHAKDTKTGFNLMKRSLSLGGLGHTAALHTTDEAVMNKFALEMTACRALINVPSSQGAIGYKYDNVAPSLTLGCGTWGHNSISHNLEDWDLLNIKTVAKRLDKIR

SEQ ID NO: 34 - DNA, 1371 bp, Lactiplantibacillus pentosus
  source 1..1371: mol_type = other DNA
atgttgaaagaaattgatgaacacaccaccgtgtcacaatcgattgatcggttagtcttgaacgcctcattagccgccactcggctagaagtgatgaaccaagaacaagttgaccaagccgtggccgccatggctcgcgcagcccacgcagctcgtggcatgttagcagccatggcagttgaggagaccggacggggaaattatcgcgataaggtcgccaagaatgatttcgcggccaaacacatttacgattacatcaaggatgacaagaccgtgggcatcattaacgacgatccggtcagtggcgtcatgaaagtggctgagccagtcggtattattgccggcgttacgccagtcactaatcccacgtcgaccgtcatcttcaacgcgatgctggccttaaagacccgtaatccgattatcttcggtttccacccgtttgcacaaaaatcatgtgtcgaaactggcaaaatcatccgtgatgccgcggttaccgcgggcgcacccaaagattggattcaatggatcaaaacgcctagtctggaagcaactaacaccttaatgaaccaccctggtgtggcaacgattattgcaaccgggggtgccggcatggtcaagacggcgtattccaccggtaagcccgcgctcggcgttggtcccggcaacgtgccctgctttatcgaacagaccgctgacgtgcatcaagccgttagtgatgtcgtcacgtccaagtcgtttgataacggcatgatttgtgcgtccgaatcgaaccttatcgtggccgaccagatttatgaccaggtcaagaccgaactcagtcaacagggcgtctatttcgttggtcctgacaattttaaggccttagaagcaaccgtcatgaacctcgataagcaggccgttgatccaaaggttgctgggcaaacaccatggcagattgcacagtgggccggtttcgacgttcctaccgacaccaaggtcttagccgttgaactaccaagcatcggtggcgaccaagtcttatcaggtgagaagttatcgccagtactcgcggtcgtacatgccaaagatactaagacgggcttcaacttgatgaaacgcagtctgtcactgggtggcttgggtcacacggccgccttgcacacgactgacgaagccgtcatgaacaagtttgccttggaaatgaccgcttgccgggcgttaatcaacgtcccatcctcacaaggggcgatcggttacaagtacgacaacgtggcgccttccctaacactcggttgcgggacatggggtcataactcgatttcacataacttggaagactgggacttgctcaatatcaagaccgtcgccaagcgtttggataaaattcgctaa

SEQ ID NO: 35 - protein, 250 aa, Lactiplantibacillus pentosus
MTGSWDARYAAEFFGTLILVLPGNGAVANAFLKNTTGNDDPGLANGGWLLVASGYGLGVMLPAMMFGSISGNHLNPAITIGQATVGLFPWSHVAQYLIWQFLGAIVGQCLILALYWPHYRQTTDAGAVLGTFATSDAAHSQLNGFITEMVGTAVLIFGAMGLYRGMFFHQNIDIANIGVGLLIAAMVISLGGPTGPALNPARDLGPRLVHALLPVPNKGSSHWEYSWVPVVAPIVGAVIGIWLYKIFFSL

SEQ ID NO: 36 - DNA, 753 bp, Lactiplantibacillus pentosus
  source 1..753: mol_type = other DNA
atgacaggttcgtgggacgcacgctacgctgcagaattctttggcaccctgatcctagtactaccaggcaacggtgctgtcgctaatgcattcctaaagaacacgaccggaaacgacgatccaggactcgccaacgggggttggttactggtggcgtcggggtacgggctgggggttatgctgccagcaatgatgttcgggtcaatctctggtaaccacttgaaccctgcgattacgattggtcaagcaacggttggtctcttcccctggtcacacgttgcgcagtacttaatctggcaatttctcggtgccatcgtaggacaatgcttgattctcgcactatactggccgcactaccgacaaacgactgatgctggcgcagtattagggacgtttgccactagtgacgccgctcacagtcagctgaacgggttcattaccgaaatggtcgggaccgccgtcctaatttttggggcgatgggcctgtatcgtgggatgttcttccatcaaaatatcgatattgccaacatcggggtcggattactgattgcggcaatggtcatctcactaggtgggccgactgggccagctctgaaccctgcccgggatttagggccgcggctcgttcacgcactattaccggttcccaataaggggtcttcacattgggaatacagctgggttccggtcgtggcaccaattgttggcgccgtgattgggatttggctttacaagatcttttttagtttataa

SEQ ID NO: 37 - protein, 238 aa, Lactiplantibacillus pentosus
MGHQLIAEFMGTALMIIFGVGVHCSSVLKGTKYRGSGHIFAITTWGFGISIALFIFGNVCINPAMVLAQCLLGNLAWSAFIPYSIAEVLGGVVGSVIVWIMYADHFKASTDEISPITIRNLFCTAPAVRNLPRNFFVELFDTFIFISGILAISEIKTPGIVPIGVGLLVWAIGMGLGGPTGFAMNLARDMGPRIAHAILPIANKADSDWQYGIIVPGIAPFVGAACAAWFMHGFFGIN

SEQ ID NO: 38 - DNA, 717 bp, Lactiplantibacillus pentosus
  source 1..717: mol_type = other DNA
ttgggacatcagctaattgccgagtttatgggcacggccctcatgattatttttggggtgggcgttcattgtagttccgtgttgaaaggcactaagtaccggggttccggtcatatttttgcgattacgacgtgggggtttgggatcagcatcgcgctgttcatttttggcaacgtttgtatcaacccggccatggtgctggcgcagtgtttgctaggaaacctggcctggtcagcgttcattccgtattcgattgccgaagtgttaggtggcgttgtcggttcagtgattgtttggatcatgtatgccgaccatttcaaagcatcgacggatgaaatctcgccaatcacgattcgaaatttattctgtaccgcgccagccgttcggaacttaccgcggaatttctttgtggaattattcgatacctttattttcatttctggtattttagcgatctctgagatcaaaacgccggggatcgtgccaattggcgttggcctgctcgtttgggcgattgggatgggactaggtggcccaactggtttcgcgatgaacttagcgcgtgacatggggccccggattgctcacgcgattttgccgattgcgaacaaagcggatagtgactggcaatatggcatcatcgttcccggcatcgcgccatttgtcggtgctgcctgtgccgcttggttcatgcatggcttttttgggattaattaa

SEQ ID NO: 39 - protein, 239 aa, Lactiplantibacillus pentosus
MKDPLALQLLGEFLGTFILILLGDGVVAGVTLNKSKAQNAGWVAITLGWGFAVTMGVYASSFMSPAHLNPAVSLGMAVAGKFPWAYVLPYSAAQIAGGIVGGLAVWLHYYPHWQATKDAGAILGIFSTGPGIRQYFWNFISEVIGTFVLVFGLLAFTKGQFTAGLNPIVVGILIIAIGLSLGGTTGYAINPARDLGPRIAHAVLPIANKGTSDWAYSWVPIAGPLVGGALGALLFNVLP

SEQ ID NO: 40 - DNA, 720 bp, Lactiplantibacillus pentosus
  source 1..720: mol_type = other DNA
atgaaagatccattagcattacaattattgggggaatttctcgggacctttatcttgattctattaggtgatggggtcgttgctggggtaaccctcaacaaatccaaagcgcaaaatgcgggctgggtcgcgattaccttaggctggggttttgccgtcacgatgggcgtctatgcgtccagcttcatgagcccggcacacttgaatcccgccgtttcgttaggtatggcggttgcgggcaagttcccatgggcatacgtcttgccttatagtgccgctcaaattgccggtgggatcgtcggtggcctggcagtctggctgcactactatccacattggcaagccacaaaggacgccggcgccattctcggaatcttctcgactggccctggtattcggcaatatttctggaacttcatcagtgaagtgattggaacctttgtcctagttttcggcttgttagccttcacgaaaggccagtttaccgcgggtttgaatccaatcgtcgtcgggattctgatcattgcgattgggttgtcactcggtggaacgaccgggtacgccatcaatccagcacgggacttgggaccacggattgcacatgccgtcttgccaatcgcaaataaaggcacttctgattgggcctacagctgggtgccaattgctggaccactggttggtggtgcgctaggtgcactgttgtttaatgtattgccataa

SEQ ID NO: 41 - protein, 238 aa, Lactiplantibacillus pentosus
MIHQLLAEFMGTALMIIFGVGVHCSEVLKGTKYRGSGHIFAITTWGFGITIALFIFGNVCINPAMVLAQCMLGNLSWSVFIPYSVAEVLGGVVGAVIVWIMYADHFAASADEISPITIRNLFSTAPAVRNLPRNFFVEFFDTFIFISGILAISEVKTPGIVPIGVGLLVWAIGMGLGGPTGFAMNLARDMGPRIAHAILPIKNKADSDWQYGIIVPGIAPFVGAACAALFMHGFFGIG

SEQ ID NO: 42 - DNA, 657 bp, Lactiplantibacillus pentosus
  source 1..657: mol_type = other DNA
ggtgtacattgtagtgaggttttaaaggggaccaagtaccgcggttccggccatatcttcgcaattaccacatggggttttgggattacgattgccctcttcattttcggcaatgtttgtattaatccagccatggtcttggctcaatgcatgttgggtaacttatcatggtcagtcttcatcccgtattcagttgcggaagtcttgggtggggtcgttggtgctgtgatcgtttggatcatgtacgctgaccattttgccgcttctgctgatgagatttcaccaattacgattcgaaacttattctcaactgcacccgctgtgcgtaacttaccacggaacttcttcgtcgaattcttcgatacctttatttttatttccggaattttagctatttctgaagtgaagacgccaggaatcgttccaattggtgtggggctcttagtttgggcgattgggatgggactcggtggtcctactggtttcgccatgaaccttgcacgggacatgggaccacggattgcccacgccatcttaccaatcaagaacaaggctgacagcgactggcaatacgggatcatcgtacccggaattgcaccattcgttggggcagcttgtgccgcattgtttatgcatgggttctttgggattgggtaa

SEQ ID NO: 43 - protein, 584 aa, Saccharomyces cerevisiae
MSAKSFEVTDPVNSSLKGFALANPSITLVPEEKILFRKTDSDKIALISGGGSGHEPTHAGFIGKGMLSGAVVGEIFASPSTKQILNAIRLVNENASGVLLIVKNYTGDVLHFGLSAERARALGINCRVAVIGDDVAVGREKGGMVGRRALAGTVLVHKIVGAFAEEYSSKYGLDGTAKVAKIINDNLVTIGSSLDHCKVPGRKFESELNEKQMELGMGIHNEPGVKVLDPIPSTEDLISKYMLPKLLDPNDKDRAFVKFDEDDEVVLLVNNLGGVSNFVISSITSKTTDFLKENYNITPVQTIAGTLMTSFNGNGFSITLLNATKATKALQSDFEEIKSVLDLLNAFTNAPGWPIADFEKTSAPSVNDDLLHNEVTAKAVGTYDFDKFAEWMKSGAEQVIKSEPHITELDNQVGDGDCGYTLVAGVKGITENLDKLSKDSLSQAVAQISDFIEGSMGGTSGGLYSILLSGFSHGLIQVCKSKDEPVTKEIVAKSLGIALDTLYKYTKARKGSSTMIDALEPFVKEFTASKDFNKAVKAAEEGAKSTATFEAKFGRASYVGDSSQVEDPGAVGLCEFLKGVQSAL

SEQ ID NO: 44 - protein, 367 aa, Escherichia coli
MDRIIQSPGKYIQGADVINRLGEYLKPLAERWLVVGDKFVLGFAQSTVEKSFKDAGLVVEIAPFGGECSQNEIDRLRGIAETAQCGAILGIGGGKTLDTAKALAHFMGVPVAIAPTIASTDAPCSALSVIYTDEGEFDRYLLLPNNPNMVIVDTKIVAGAPARLLAAGIGDALATWFEARACSRSGATTMAGGKCTQAALALAELCYNTLLEEGEKAMLAAEQHVVTPALERVIEANTYLSGVGFESGGLAAAHAVHNGLTAIPDAHHYYHGEKVAFGTLTQLVLENAPVEEIETVAALSHAVGLPITLAQLDIKEDVPAKMRIVAEAACAEGETIHNMPGGATPDQVYAALLVADQYGQRFLQEWE

SEQ ID NO: 45 - protein, 440 aa, Catonella morbi
MSELFQNIPKIKYEGANSKNPLAFHYYDAEKIVLGKTMKEHLPFAMAWWHNLCAAGTDMFGRDTADKSFGLEKGSMEHAKAKVDAGFEFMEKLGIKYFCFHDVDLVPEACDIKETNSRLDEISDYILEKMKGTDIKCLWGTANMFSNPRFVNGAGSTNSADVYCFAAAQIKKALDITVKLGGRGYVFWGGREGYETLLNTDVKFEQENIANLMKMAVEYGRSIGFKGDFYIEPKPKEPMKHQYDFDAATAIGFLRQYGLDKDFKLNIEANHATLAGHSFQHELRISSINGMLGSVDANQGDMLLGWDTDEFPFDVYDTTMCMYEVLKNGGLTGGFNFDAKNRRPSYTYEDMFYGFILGMDSFALGLIKAAKLIEEGTLDNFIKERYKSFESEIGKKIRSKSASLQELAAYAEEMGAPAMPGSGRQEYLQAALNQNLFGEV

SEQ ID NO: 46 - protein, 600 aa, Saccharomyces cerevisiae
MLCSVIQRQTREVSNTMSLDSYYLGFDLSTQQLKCLAINQDLKIVHSETVEFEKDLPHYNTKKGVYIHGDAIECPVAMWLEALDLVLSKYREAKFPLNKVMAVSGSCQQHGSVYWSSQAESLLEQLNKKPEKDLLHYVSSVAFARQTAPNWQDHSTAKQCQEFEECIGGPEKMAQLTGSRAHFRFTGPQILKIAQLEPEAYEKTKTISLVSNFLTSILMGHLVELEEADACGMNLYDIRERKFSDELLHLIDSSSKDKTIRQKLMRAPMKNLIAGTICKYFIEKYGFNTNCKVSPMTGDNLATICSLPLRKNDVLVSLGTSTTVLLVTDKYHPSPNYHLFIHPTLPNHYMGMICYCNGSLARERIRDELNKERENNYEKTNDWILFNQAVLDDSESSENELGVYFPLGEIVPSVKAINKRVIFNPKTGMIEREVAKFKDKRHDAKNIVESQALSCRVRISPLLSDSNASSQQRLNEDTIVKFDYDESPLRDYLNKRPERTFFVGGASKNDAIVKKFAQVIGATKGNFRLETPNSCALGGCYKAMWSLLYDSNKIAVPFDKFLNDNFPWHVMESISDVDNENWDRYNSKIVPLSELEKTLI

SEQ ID NO: 47 - protein, 680 aa, Saccharomyces cerevisiae
MTQFTDIDKLAVSTIRILAVDTVSKANSGHPGAPLGMAPAAHVLWSQMRMNPTNPDWINRDRFVLSNGHAVALLYSMLHLTGYDLSIEDLKQFRQLGSRTPGHPEFELPGVEVTTGPLGQGISNAVGMAMAQANLAATYNKPGFTLSDNYTYVFLGDGCLQEGISSEASSLAGHLKLGNLIAIYDDNKITIDGATSISFDEDVAKRYEAYGWEVLYVENGNEDLAGIAKAIAQAKLSKDKPTLIKMTTTIGYGSLHAGSHSVHGAPLKADDVKQLKSKFGFNQDKSFVVPQEVYDHYQKTILKPGVEANNKWNKLFSEYQKKFPELGAELARRLSGQLPANWESKLPTYTAKDSAVATRKLSETVLEDVYNQLPELIGGSADLTPSNLTRWKEALDFQPPSSGSGNYSGRYIRYGIREHAMGAIMNGISAFGANYKPYGGTFLNFVSYAAGAVRLSALSGHPVIWVATHDSIGVGEDGPTHQPIETLAHFRSLPNIQVWRPADGNEVSAAYKNSLESKHTPSIIALSRQNLPQLEGSSIESASKGGYVLQDVANPDIILVATGSEVSLSVEAAKTLAAKNIKARVVSLPDFFTFDKQPLEYRLSVLPDNVPIMSVEVLATTCWGKYAHQSFGIDRFGASGKAPEVFKFFGFTPEGVAERAQKTIAFYKGDKLISPLKKAF

SEQ ID NO: 48 - protein, 681 aa, Saccharomyces cerevisiae
MAQFSDIDKLAVSTLRLLSVDQVESAQSGHPGAPLGLAPVAHVIFKQLRCNPNNEHWINRDRFVLSNGHSCALLYSMLHLLGYDYSIEDLRQFRQVNSRTPGHPEFHSAGVEITSGPLGQGISNAVGMAIAQANFAATYNEDGFPISDSYTFAIVGDGCLQEGVSSETSSLAGHLQLGNLITFYDSNSISIDGKTSYSFDEDVLKRYEAYGWEVMEVDKGDDDMESISSALEKAKLSKDKPTIIKVTTTIGFGSLQQGTAGVHGSALKADDVKQLKKRWGFDPNKSFVVPQEVYDYYKKTVVEPGQKLNEEWDRMFEEYKTKFPEKGKELQRRLNGELPEGWEKHLPKFTPDDDALATRKTSQQVLTNMVQVLPELIGGSADLTPSNLTRWEGAVDFQPPITQLGNYAGRYIRYGVREHGMGAIMNGISAFGANYKPYGGTFLNFVSYAAGAVRLAALSGNPVIWVATHDSIGLGEDGPTHQPIETLAHLRAIPNMHVWRPADGNETSAAYYSAIKSGRTPSVVALSRQNLPQLEHSSFEKALKGGYVIHDVENPDIILVSTGSEVSISIDAAKKLYDTKKIKARVVSLPDFYTFDRQSEEYRFSVLPDGVPIMSFEVLATSSWGKYAHQSFGLDEFGRSGKGPEIYKLFDFTADGVASRAEKTINYYKGKQLLSPMGRAF

SEQ ID NO: 49 - protein, 335 aa, Saccharomyces cerevisiae
MSEPAQKKQKVANNSLEQLKASGTVVVADTGDFGSIAKFQPQDSTTNPSLILAAAKQPTYAKLIDVAVEYGKKHGKTTEEQVENAVDRLLVEFGKEILKIVPGRVSTEVDARLSFDTQATIEKARHIIKLFEQEGVSKERVLIKIASTWEGIQAAKELEEKDGIHCNLTLLFSFVQAVACAEAQVTLISPFVGRILDWYKSSTGKDYKGEADPGVISVKKIYNYYKKYGYKTIVMGASFRSTDEIKNLAGVDYLTISPALLDKLMNSTEPFPRVLDPVSAKKEAGDKISYISDESKFRFDLNEDAMATEKLSEGIRKFSADIVTLFDLIEKKVTA

SEQ ID NO: 50 - protein, 489 aa, Saccharomyces cerevisiae
MSADFGLIGLAVMGQNLILNAADHGFTVCAYNRTQSKVDHFLANEAKGKSIIGATSIEDFISKLKRPRKVMLLVKAGAPVDALINQIVPLLEKGDIIIDGGNSHFPDSNRRYEELKKKGILFVGSGVSGGEEGARYGPSLMPGGSEEAWPHIKNIFQSISAKSDGEPCCEWVGPAGAGHYVKMVHNGIEYGDMQLICEAYDIMKRLGGFTDKEISDVFAKWNNGVLDSFLVEITRDILKFDDVDGKPLVEKIMDTAGQKGTGKWTAINALDLGMPVTLIGEAVFARCLSALKNERIRASKVLPGPEVPKDAVKDREQFVDDLEQALYASKIISYAQGFMLIREAAATYGWKLNNPAIALMWRGGCIIRSVFLGQITKAYREEPDLENLLFNKFFADAVTKAQSGWRKSIALATTYGIPTPAFSTALSFYDGYRSERLPANLLQAQRDYFGAHTFRVLPECASDNLPVDKDIHINWTGHGGNVSSSTYQA

SEQ ID NO: 51 - protein, 238 aa, Saccharomyces cerevisiae
MVKPIIAPSILASDFANLGCECHKVINAGADWLHIDVMDGHFVPNITLGQPIVTSLRRSVPRPGDASNTEKKPTAFFDCHMMVENPEKWVDDFAKCGADQFTFHYEATQDPLHLVKLIKSKGIKAACAIKPGTSVDVLFELAPHLDMALVMTVEPGFGGQKFMEDMMPKVETLRAKFPHLNIQVDGGLGKETIPKAAKAGANVIVAGTSVFTAADPHDVISFMKEEVSKELRSRDLLD

SEQ ID NO: 52 - protein, 508 aa, Bacteroides thetaiotaomicron
MNNVFDQYEVWFVTGAQLLYGGDAVIAVDAHSNEMVNGLNESGKLPVKVVYKGTANSSKEVEAVFKAANNDDKCVGVITWMHTFSPAKMWIHGLQQLKKPLLHLHTQFNKEIPWDTMDMDFMNLNQSAHGDREFGHICTRMRIRRKVVVGYWKEEETLHKIAVWMRVCAGWADSQDMLIIRFGDQMNNVAVTDGDKVEAEQRMGYHVDYCPASELMEYHKDIKNADVDALVATYFNDYDHDASLEDKSTEAYQKVWNAAKAELALRAILKAKGAKGFTTNFDDLGQTDGSYFDQIPGLASQRLMAEGYGFGAEGDWKSAALYRTVWVMNQGLPKGCSFLEDYTLNFDGANSSILQSHMLEICPLIAANKPRLEVHFLGIGIRKSQTARLVFTSKTGTGCTATVVDMGNRFRLIVNDVECIEPKPLPKLPVASALWIPMPNLEVGAGAWILAGGTHHSCFSYDLTAEYWEDYAEIAGIEMVHINKDTTISCFKKELRMNEVYYMLNKAL

SEQ ID NO: 53 - protein, 531 aa, Bacteroides thetaiotaomicron
MKLDAKSTIETGKAILGIELGSTRIKAVLIDQENKPIAQGSHTWENQLVNGLWTYSIDAIWSGLQDCYADLRSNVKKLYDTEIETLAAIGVSAMMHGYMPFNEKEEILVPFRTWRNTNTGRAAAELSELFVYNIPLRWSISHLYQAILDNEAHVKDIKFLTTLAGYVHWQITGEKVLGIGDASGMLPIDPTTNNYSAEMVAKFNNLIASKEYSWKLEDILPKVLSAGENAGVLTPEGCKKLDASGHLKAGIPVCPPEGDAGTGMVATNAVKQRTGNVSAGTSSFSMIVLEKELSKPYEMIDMVTTPDGSLVAMVHCNNCTSDLNAWVNLFKEYQELLGIPVDMDELYGKLYNIALTGDTDCGGLLSYNYISGEPVTGLAEGRPLFVRSANDKFNLANFMRAHLYASVGVLKIGNDILFNEEKIKVDRITGHGGLFRTKGVGQRVLAAAINSPISVMETAGEGGAWGIALLGSYLVNNKKGQSLADFLDESVFVSDAGVEVSPTPEDVAGFNTYIESYKAGLPIEEAAVKFK

SEQ ID NO: 54 - protein, 227 aa, Bacteroides thetaiotaomicron
MLEELKEKVFHANLELVKHGLVIFTWGNVSAIDRETELVVIKPSGVSYDDMKAEDMVVVDLDGKVVEGRLKPSSDTPTHVVLYKAFPEIGGVVHTHSTYATAWAQAGCDIPNIGTTHADYFHDAIPCTADMTEAEVKGAYELETGNVIVKRFEGLNPVHTPGVLVKNHGPFSWGKDAHDAVHNAVVMEQVAKMASIAYAVNPNLTMNPLLVEKHFSRKHGPNAYYGQ

SEQ ID NO: 55 - protein, 910 aa, Bifidobacterium adolescentis
MADAKKKEEPTKPTPEEKLAAAEAEVDALVKKGLKALDEFEKLDQKQVDHIVAKASVAALNKHLVLAKMAVEETHRGLVEDKATKNIFACEHVTNYLAGQKTVGIIREDDVLGIDEIAEPVGVVAGVTPVTNPTSTAIFKSLIALKTRCPIIFGFHPGAQNCSVAAAKIVRDAAIAAGAPENCIQWIEHPSIEATGALMKHDGVATILATGGPGMVKAAYSSGKPALGVGAGNAPAYVDKNVDVVRAANDLILSKHFDYGMICATEQAIIADKDIYAPLVKELKRRKAYFVNADEKAKLEQYMFGCTAYSGQTPKLNSVVPGKSPQYIAKAAGFEIPEDATILAAECKEVGENEPLTMEKLAPVQAVLKSDNKEQAFEMCEAMLKHGAGHTAAIHTNDRDLVREYGQRMHACRIIWNSPSSLGGVGDIYNAIAPSLTLGCGSYGGNSVSGNVQAVNLINIKRIARRNNNMQWFKIPAKTYFEPNAIKYLRDMYGIEKAVIVCDKVMEQLGIVDKIIDQLRARSNRVTFRIIDYVEPEPSVETVERGAAMMREEFEPDTIIAVGGGSPMDASKIMWLLYEHPEISFSDVREKFFDIRKRAFKIPPLGKKAKLVCIPTSSGTGSEVTPFAVITDHKTGYKYPITDYALTPSVAIVDPVLARTQPRKLASDAGFDALTHAFEAYVSVYANDFTDGMALHAAKLVWDNLAESVNGEPGEEKTRAQEKMHNAATMAGMAFGSAFLGMCHGMAHTIGALCHVAHGRTNSILLPYVIRYNGSVPEEPTSWPKYNKYIAPERYQEIAKNLGVNPGKTPEEGVENLAKAVEDYRDNKLGMNKSFQECGVDEDYYWSIIDQIGMRAYEDQCAPANPRIPQIEDMKDIAIAAYYGVSQAEGHKLRVQRQGEAATEEASERA

SEQ ID NO: 56 - protein, 683 aa, Saccharomyces cerevisiae
MTIKEHKVVYEAHNVKALKAPQHFYNSQPGKGYVTDMQHYQEMYQQSINEPEKFFDKMAKEYLHWDAPYTKVQSGSLNNGDVAWFLNGKLNASYNCVDRHAFANPDKPALIYEADDESDNKIITFGELLRKVSQIAGVLKSWGVKKGDTVAIYLPMIPEAVIAMLAVARIGAIHSVVFAGFSAGSLKDRVVDANSKVVITCDEGKRGGKTINTKKIVDEGLNGVDLVSRILVFQRTGTEGIPMKAGRDYWWHEEAAKQRTYLPPVSCDAEDPLFLLYTSGSTGSPKGVVHTTGGYLLGAALTTRYVFDIHPEDVLFTAGDVGWITGHTYALYGPLTLGTASIIFESTPAYPDYGRYWRIIQRHKATHFYVAPTALRLIKRVGEAEIAKYDTSSLRVLGSVGEPISPDLWEWYHEKVGNKNCVICDTMWQTESGSHLIAPLAGAVPTKPGSATVPFFGINACIIDPVTGVELEGNDVEGVLAVKSPWPSMARSVWNHHDRYMDTYLKPYPGHYFTGDGAGRDHDGYYWIRGRVDDVVNVSGHRLSTSEIEASISNHENVSEAAVVGIPDELTGQTVVAYVSLKDGYLQNNATEGDAEHITPDNLRRELILQVRGEIGPFASPKTIILVRDLPRTRSGKIMRRVLRKVASNEAEQLGDLTTLANPEVVPAIISAVENQFFSQKKK

SEQ ID NO: 57 - protein, 569 aa, Saccharomyces paradoxus
MKDSKLSKFKGRFMSRTSHWGLTGQKLRYFITIASMTGFSLFGYDQGLMASLITGKQFNYEFPATKENGDHDRHATVVQGATTSCYELGCFAGSLFVMFYGERIGRKPLILMGSIITIIGAVISTCAFRDYWALGQFIVGRVVTGVGTGLNTSTIPVWQSEMSKAENRGLLVNLEGSTIAFGTMIAYWIDFGFSYTNSSVQWRFPVSMQIVFALFLLAFMIKLPESPRWLISQSRTEEARYLVGTLDDTDPNDEEVITEVAMLHDAVNRTKHEKHSLSSLFSRGKSQNLQRALIAASTQFFQQFTGCNAAIYYSTVLFNKTIKLDHRLSMIIGGVFATIYALSTIGSFFLIEKLGRRKLFLLGATGQAVSFTITFACLVKENKENARGAAVGLFLFITFFGLSLLSLPWIYPPEIASMKVRASTNAFSTCTNWLCNFAVVMFTPIFIGQSGWGCYLFFAVMNYLYIPVIFFFYPETAGRSLEEIDIIFAKAYEDGTQPWRVANHLPKLSLQEVEDHANALGSYDDEMEKDDFAEDRVEDTYNQINGDNSSSSSNIKNEDTVNDKANSES

SEQ ID NO: 58 - protein, 571 aa, Millerozyma farinosa
MGFELWGRTNTGGLRGRPLRVAITAVATTGFSLFGYDQGLMSGIITGTEFNEEFPPTWSKPHYNASEKRHATVVQGAVTACYEIGCFFGALFALVRGDRIGRRPLVIVGAVLIIIGTVISTAAFGEHWGLGQFVIGRVITGIGNGMNTATIPVWQSEISRPENRGKLVNLEGSVIAIGTFVAYWIDFGLSYVNSSVQWRFPVAFQIVFAAGLLGGILFMPESPRWLLAHGKKEQAHIVLGALNDLDPNDDHVLAESTVITDAINRFSRSQLGFKELMSGGKNQHFARMVIGSSTQFFQQFTGCNAAIYYSTVLFEETIFVGDRRLSLVMGGVFASVYALATIPSFFLVDKLGRRNLFLIGATGQALSFTITFACLINPTKQNAKGAAVGIFLFITFFAFTILPLPWIYPPEINPLRTRTVASAVSTCTNWLTNFAVVMFTPIFINDAQWGCYLFFACLNYAFIPVIFWFYPETAGRSLEEIDIIFAKAYTDGRPPWRVAATMPHLSLKEQEEQGMQLGLYDNEAEKQKFEQTENLMSSSSSAKLPEEGSNVNENENENTNEKDQTPKPTDV

SEQ ID NO: 59 - protein, 569 aa, Saccharomyces cerevisiae
MKDLKLSNFKGKFISRTSHWGLTGKKLRYFITIASMTGFSLFGYDQGLMASLITGKQFNYEFPATKENGDHDRHATVVQGATTSCYELGCFAGSLFVMFCGERIGRKPLILMGSVITIIGAVISTCAFRGYWALGQFIIGRVVTGVGTGLNTSTIPVWQSEMSKAENRGLLVNLEGSTIAFGTMIAYWIDFGLSYTNSSVQWRFPVSMQIVFALFLLAFMIKLPESPRWLISQSRTEEARYLVGTLDDADPNDEEVITEVAMLHDAVNRTKHEKHSLSSLFSRGRSQNLQRALIAASTQFFQQFTGCNAAIYYSTVLFNKTIKLDYRLSMIIGGVFATIYALSTIGSFFLIEKLGRRKLFLLGATGQAVSFTITFACLVKENKENARGAAVGLFLFITFFGLSLLSLPWIYPPEIASMKVRASTNAFSTCTNWLCNFAVVMFTPIFIGQSGWGCYLFFAVMNYLYIPVIFFFYPETAGRSLEEIDIIFAKAYEDGTQPWRVANHLPKLSLQEVEDHANALGSYDDEMEKEDFGEDRVEDTYNQINGDNSSSSSNIKNEDTVNDKANFEG

SEQ ID NO: 60 - protein, 574 aa, Saccharomyces cerevisiae
MAVEENNMPVVSQQPQAGEDVISSLSKDSHLSAQSQKYSNDELKAGESGPEGSQSVPIEIPKKPMSEYVTISLLCLCVAFGGFMFGWDTGTISGFVVQTDFLRRFGMKHKDGTHYLSNVRTGLIVAIFNIGCAFGGIILSKGGDMYGRKKGLSIVVSVYIVGIIIQIASINKWYQYFIGRIISGLGVGGIAVLCPMLISEIAPKHLRGTLVSCYQLMITAGIFLGYCTNYGTKSYSNSVQWRVPLGLCFAWSLFMIGALTLVPESPRYLCEVNKVEDAKRSIAKSNKVSPEDPAVQAELDLIMAGIEAEKLAGNASWGELFSTKTKVFQRLLMGVFVQMFQQLTGNNYFFYYGTVIFKSVGLDDSFETSIVIGVVNFASTFFSLWTVENLGRRKCLLLGAATMMACMVIYASVGVTRLYPHGKSQPSSKGAGNCMIVFTCFYIFCYATTWAPVAWVITAESFPLRVKSKCMALASASNWVWGFLIAFFTPFITSAINFYYGYVFMGCLVAMFFYVFFFVPETKGLSLEEIQELWEEGVLPWKSEGWIPSSRRGNNYDLEDLQHDDKPWYKAMLE

SEQ ID NO: 61 - protein, 574 aa, Saccharomyces cerevisiae
MAVEENNMPVVSQQPQAGEDVISSLSKDSHLSAQSQKYSNDELKAGESGSEGSQSVPIEIPKKPMSEYVTVSLLCLCVAFGGFMFGWDTGTISGFVVQTDFLRRFGMKHKDGTHYLSNVRTGLIVAIFNIGCAFGGIILSKGGDMYGRKKGLSIVVSVYIVGIIIQIASINKWYQYFIGRIISGLGVGGIAVLCPMLISEIAPKHLRGTLVSCYQLMITAGIFLGYCTNYGTKSYSNSVQWRVPLGLCFAWSLFMIGALTLVPESPRYLCEVNKVEDAKRSIAKSNKVSPEDPAVQAELDLIMAGIEAEKLAGNASWGELFSTKTKVFQRLLMGVFVQMFQQLTGNNYFFYYGTVIFKSVGLDDSFETSIVIGVVNFASTFFSLWTVENLGHRKCLLLGAATMMACMVIYASVGVTRLYPHGKSQPSSKGAGNCMIVFTCFYIFCYATTWAPVAWVITAESFPLRVKSKCMALASASNWVWGFLIAFFTPFITSAINFYYGYVFMGCLVAMFFYVFFFVPETKGLSLEEIQELWEEGVLPWKSEGWIPSSRRGNNYDLEDLQHDDKPWYKAMLE

SEQ ID NO: 62 - protein, 806 aa, Aspergillus niger
MPGEVIDRPNPKAEPSHIPDLVNQLQVKLQETRLEETDYNALLKFRRAAAYIAAAMIFLQDNVLLKQNLRHEDIKPRLLGHWGTCPGLILVYSHLNYIIRKQNLDMLYVVGPGHGAPAILASLWLEGSLEKFYPHYSRDMDGLHELISTFSTSAGLPSHINAETPGAIHEGGELGYALAVSFGAVMDNPDMIVTCVVGDGEAETGPTATSWHAIKYIDPAESGAVLPILHVNGFKISERTIYGCMDNKELVSLFTGYGYQVRIVENLDDIDADLHSSMMWAVEEIHKIQKAARSGKPIMKPRWPMIVLRTPKGWSGPKELHGSFIEGSFHSHQVPLPNAKKDKEELQALQKWLSSYNPHELFTETGDIIDDIKSVIPLEDTKKLGQRAEAYKGYRAPDLPDWRKFGVEKGSQQSAMKTIGKFIDQVFTQNPHGVRVFSPDELESNKLDAALAHTGRNFQWDQFSNAKGGRVIEVLSEHLCQGFMQGYTLTGRVGIFPSYESFLGIIHTMMVQYAKFNKMAQETTWHKPVSSINYIETSTWARQEHNGFSHQNPSFIGAVLRLKPTAARVYLPPDANTFLTTLHHCLKSKNYVNLMVGSKQPTPVYLSPEEAESHCRAGASIWRFCSTDNGLNPDVVLVGIGVEVMFEVIYAAAILRKRCPELRVRVVNVTDLMILEKEGLHPHALTTEAFDSLFGSDRPIHFNYHGYPGELKGLLFGRPRLDRVSVEGYMEEGSTTTPFDMMLLNRVSRYHVAQAAVIGASRRNEKVQVRQHELVSEFGHNIVETRKYILANRKDPDDTYDMPSFE

SEQ ID NO: 63 - protein, 815 aa, Neurospora crassa
MGGTQITIPNPPPLPSHLPDNVLDLAVQIERKNLPDNVRESLRAFQRAGCYIAASMIFLRDNVLLQDKLQVSHIKPRLLGHWGTCPGLILVWSHLNVLIRNHDLNMIYVIGPGHGAPAALASLWLEGSLERFYPGEYDRNATGLRNLITRFSVPGGFPSHINAETPGAIHEGGELGYALSVSFGAVMDNPDLIVTCVVGDGEAETGPTATAWHAIKYIDPKESGAVIPILHVNGFKISERTIFGCMDDKEIACLFSGYGYQVRIVEDLENIDDDLQSSLEWALVEIKKIQKAAREDNKPIVKPRWPMIVLRTPKGWTGPKEVEGQIIEGSFHSHQVPLPKAGSDEKQLQALDEWLSSYRISELLDDEGKPTEAITRVLPKAEKRLGQLKITYDPYIPLKLIDWKQFGVQKGVDESCMKSAGKFLDKLFQENPKTLRLFSPDELESNKLNAILDHTQRDFQWDEFSRAHGGRVIEVLSEHNCQGFMQGYTLTGRTALFPSYESFLGIVHTMMVQYSKFVKIAREVPWRGDLASINYIETSTWARQEHNGFSHQNPSFIGAVLNLKAEAARVYLPPDANCFLSTVHHVMDSKNKTNLIIGSKQPTAVYLSPAEAADHCRQGASIWHFASSSDPNSSAGESVQDPDVVLVGIGVEVTFEVIKAAELLRSIAPALKIRVVNVTDLMILIEESKHPHALAKAKFIEMFTADRPVLFNYHGYPTELQGLLFGREKAERMDVEGYQEEGSTTTPFDMMLRNRVSRFDVASWALKRGAERNAEVKNDLEGLLGDVDRRVKEVREFIGNEGKDPDDIYQLPKFE

SEQ ID NO: 64 - protein, 793 aa, Lacticaseibacillus paracasei
MDTKVKTVDYSSKEYFDKMTAYWRAANYISVGQLYLKDNPLLERPLKSEDVKPHPIGHWGTIAGQNFIYTHLNRVINKYDLNMFYIEGPGHGGQVMVSNSYLDGSYSEIYPRVSQDKEGMKNLFTQFSWPGGVASHASAQTPGSIHEGGELGYALSHATGAILDNPEVIAAVVTGDGETETGPLAASWFSNTFINPITDGAILPIVHMNGFKISNPTILSRKSDEDLDKYFSGMGWKPFFVEGDDPEKLNPEMAKVLDEAIEDIKAIQKHARETGDTTMPHWPVIIFRSPKGWTGPKSWNGEPIEGSFRAHQIPIPVDAEDMEHADSLAGWLKSYHPEELFDENGKLIPELAALPPKGEQRMAANPITNGGIDPKPLVLPDYRKYALDNKEHGKQIKQDMIVWSDYLRDLIKLNPHNFRIFGPDETMSNRLYSLFEVTNRQWLEPIKEPADQYLAPAGRIIDSQLSEHQAEGFNEGYTLTGRHGLFTSYEAFLRVVDSMLTQHFKWIRKAHEEPWHKAYPSLNVVSTSTSFQQDHNGYTHQDPGILTHMAEKKAEYIREYLPADANSLLAISPKLFSSQNTVNVLITSKQPRPQFYSIDEATVLANSGLKRIDWASNDDGVEPDVVIAAAGTEPNMESLAAINLLHDAFPDLKIRFINVVDLLKLRSPEIDPRGLSDAEFNSYFTTDKPILFAYHGFEGLIRDIFFTRPNRNVLIHGYREEGDITTPFDMRVLNELDRYHLAKDVIQHVPAYAEKAAAFVQKMDDTLQYHHDFIRANGDDIPEVQEWTWKPIK

SEQ ID NO: 65 - protein, 801 aa, Bifidobacterium bifidum
MDKYWRVANYLSIGQIYLRSNPLMKEPFTREDVKHRLVGHWGTTPGLNFLIGHINRFIADHGQNTVFIMGPGHGGPAGTSQSYLDGTYTETYPNITKDEAGLQKFFRQFSYPGGIPSHFAPETPGSIHEGGELGYALSHAYGAIMDNPSLFVPCIVGDGEAETGPLATGWQSNKLVNPRTDGIVLPILHLNGYKIANPTILSRISDEELHEFFHGMGYEPYEFVAGFDDEDHMSIHRRFAELFESVWDEICDIKAAANTDNMHRPFYPMIIFRTPKGWTCPKYIDGKKTEGSWRAHQVPLASARDTEAHFEVLKNWLESYKPEELFDANGAVKDDVLAFMPKGELRIGANPNANGGVIRRDLVLPALEDYEVKEVKEFGHGWGQLEATRRLGVYTRDIIKNNMHDFRIFGPDETASNRLQASYEVTNKQWDAGYISDEVDEHMHVSGQVVEQLSEHQMEGFLEAYLLTGRHGIWSSYESFVHVIDSMLNQHAKWLEATVREIPWRKPIASMNLLVSSHVWRQDHNGFSHQDPGVTSVLLNKCFHNDHVIGIYFATDANMLLAIAEKCYKSTNKINAIIAGKQPAATWLTLDEARAELAKGAAAWDWASTAKTNDEAQVVLAAAGDVPTQEIMAASDKLKALGIKFKVVNVADLLSLQSAKENDEALTDEEFADIFTADKPVLFAYHSYAHDVRGLIYDRPNHDNFNVHGYEEEGSTTTPYDMVRVNELDRYELTAEALRMIDADKYADEIQKLEDFRQEAFQFAVDKGYDHPDYTDWVYSGVKTDKKGAVTATAATAGDNE

SEQ ID NO: 66 - protein, 826 aa, Bifidobacterium gallicum
MTSPVIGTPWQKLNRPVSEEAIEGMDKYWRASNYMSIGQIYLRSNPLMKEPFTRDDVKYRLVGHWGTTPGLNFLLAHINRLIADHQQNTVFIMGPGHGGPAGTAQSYLDGTYTEYYPNITKDEEGLQKFFRQFSYPGGIPSHFAPETPGSIHEGGELGYALSHAYGAVMNNPSLFVPCIVGDGEAETGPLATGWQSNKLVNPRTDGIVLPILHLNGYKIANPTILARVSDEELHDFFRGLGYHPYEFVAGFDNEDHLSIHRRFAELFETIFDEICDIKAAANTDDMTRPFYPMLIFRTPKGWTCPKFIDGKKTEGSWRAHQVPLASARDTEAHFEVLKNWMASYKPEELFDDKGAIKDDVVDFMPKGDLRIGANPNANGGVIREELDLPALENYEVKEVKEFGHGWGQLEATRKLGEYTRDIIKNNPDSFRIFGPDETASNRLQASYEVTNKQWDNGYLSKDLVDEHMAVTGQVTEQLSEHQCEGFLEAYLLTGRHGIWSSYESFVHVIDSMLNQHAKWLEATVREIPWRKPISSMNLLVSSHVWRQDHNGFSHQDPGVTSVLLNKTFNNDHVIGLYFATDANVLLAIAEKCYKSTNMINAIVAGKQPAATWTTLDEARELVAKGAGEFEWASNVKTNDEAEIVLASAGDVPTQELMAAADRLNKLGVKFKVVNVVDLIKLQSAKENDQALSDAEFAELFTEDKPVLFAYHSYAHDVRGLIFDRPNHDNFNVVGYKEQGSTTTPYDMVRVNDIDRYELTATALRMIDADKYADEIKKLEDFRIEAYQFAVDNGYDIPDYTDWVWPGVKTDLPGAVSATAATAGDNE

SEQ ID NO: 67 - protein, 825 aa, Bifidobacterium animalis
MTNPVIGTPWQKLDRPVSEEAIEGMDKYWRVANYMSIGQIYLRSNPLMKEPFTRDDVKHRLVGHWGTTPGLNFLLAHINRLIADHQQNTVFIMGPGHGGPAGTAQSYIDGTYTEYYPNITKDEAGLQKFFRQFSYPGGIPSHFAPETPGSIHEGGELGYALSHAYGAIMNNPSLFVPCIIGDGEAETGPLATGWQSNKLVNPRTDGIVLPILHLNGYKIANPTLLARISDEELHDFFRGMGYHPYEFVAGFDNEDHLSIHRRFAELFETIFDEICDIKAAAQTDDMTRPFYPMLIFRTPKGWTCPKFIDGKKTEGSWRAHQVPLASARDTEAHFEVLKGWMESYKPEELFNADGSIKDDVTAFMPKGELRIGANPNANGGRIREDLKLPELDQYEITGVKEYGHGWGQVEAPRSLGAYCRDIIKNNPDSFRIFGPDETASNRLNATYEVTKKQWDNGYLSALVDENMAVTGQVVEQLSEHQCEGFLEAYLLTGRHGMWSTYESFAHVIDSMLNQHAKWLEATVREIPWRKPISSVNLLVSSHVWRQDHNGFSHQDPGVTSVLINKTFNNDHVTNIYFATDANMLLAIAEKCFKSTNKINAIFSGKQPAPTWITLDEARAELEAGAAEWKWASNAKSNDEVQIVLAAAGDVPTQEIMAASDALNKDGIKFKVVNVVDLLKLQSPENNDEAMSNEDFTELFTADKPVLFAYHSYAQDVRGLIYDRPNHDNFNVVGYKEQGSTTTPFDMVRVNDMDRYALEAQALELIDADKYADKIDELNAFRKTAFQFAVDNGYDIPEFTDWVYPDVKVDETQMLSATAATAGDNE

SEQ ID NO: 68 - protein, 788 aa, Lactiplantibacillus pentosus
MSTDYSSPAYLQKVDKYWRAANYLSVGQLYLKDNPLLQRPLKASDVKVHPIGHWGTIAGQNFIYAHLNRVINKYGLKMFYVEGPGHGGQVMVSNSYLDGTYTDIYPEITQDVEGMQKLFKQFSFPGGVASHAAPETPGSIHEGGELGYSISHGVGAILDNPDEIAAVVVGDGESETGPLATSWQSTKFINPINDGAVLPILNLNGFKISNPTIFGRTSDEKIKQYFESMNWEPIFVEGDDPEKVHPALAKAMDEAVEKIKAIQKNARENDDATLPVWPMIVFRAPKGWTGPKSWDGDKIEGSFRAHQIPIPVDQTDMEHADALVDWLESYQPKELFNEDGSLKDDIKEIIPTGDARMAANPITNGGVDPKALNLPNFRDYAVDTSKHGANVKQDMIVWSDYLRDVIKKNPDNFRLFGPDETMSNRLYGVFETTNRQWMEDIHPDSDQYEAPAGRVLDAQLSEHQAEGWLEGYVLTGRHGLFASYEAFLRVVDSMLTQHFKWLRKANELDWRKKYPSLNIIAASTVFQQDHNGYTHQDPGALTHLAEKKPEYIREYLPADANSLLAVGDVIFRSQEKINYVVTSKHPRQQWFSIEEAKQLVDNGLGIIDWASTDQGSEPDIVFAAAGTEPTLETLAAIQLLHDSFPDMKIRFVNVVDILKLRSPEKDPRGLSDAEFDHYFTKDKPVVFAFHGYEDLVRDIFFDRHNHNLHVHGYRENGDITTPFDVRVMNQMDRFDLAKSAIAAQPAMENTGAAFVQDMDNMLAKHNAYIRDAGTDLPEVNDWQWKGLK

SEQ ID NO: 69 - protein, 799 aa, Lactobacillus acidophilus
MTVNYDSKDYLKSVDAYWRAANYLSVGQLFLMKNPLLKKPLTAEDVKPKPIGHWGTIAPQNFIYAHLNRALKKYDLDMFYIEGSGHGGQVMVSNSYLDGSYTERYPEITQDEKGMAKLFKRFSFPGGVASHAAPETPGSIHEGGELGYALSHGVGAILDNPDVIAAVEIGDGEAETGPLAASWFSDKFINPIKDGAVLPILQINGFKISNPTIVSRMSDEELTEYFRGMGWDPHFVSVFKGGRFDGEKDPMQVHEEMAKTMDEVIEEIKAIQKHARENNDATLPHWPLIIFQCPKGWTGPKKDLDGNPIENSFRAHQIPIPVSQYDMKHVDMLTDWLESYKPNELFNEDGSPKEIVTENTAKGDQRMAMNPITNGGKDPKRLNLPDYRNFALKFDKPGSVEAQDMVEWAKYLNEVAKLNPTTFRGFGPDESKSNRLFKLLDDQKRQWEPEVHEPNDENLAPSGRVIDSQLSEHQDEGFLEGYVLTGRHGFFATYEAFGRVVDSMLTQHMKWLRKAKEQYWRHDYPSLNFVATSTVFQQDHNGYTHQDPGILTHLYEKNRPDLVHEYLPSDTNTLLAVGNKAFTDRECINVLVTSKQPRPQWFSIEEAQKLVDKGLSYIDWASTDKGVKPDIVFASTETEPTIETLAAIDILHDKFPDLKIRYINVIDVMKLMSPKDNKNGISDEEFDRLFPKDVPVIFAWHGYKSMMESIWFARNRHNVHIHCYEENGDITTPFDMRVLNHLDRFDLAKDAVESVDKLKGKNADFISHMDDLLEKHHQYIRDNGKDMPEVTEWKWKGLK

SEQ ID NO: 70 - protein, 822 aa, Penicillium chrysogenum
MPAEVISEPNPQALPSHLPDYLEKLSVSLEREKLDEKTYDALIKFRRAACYIAAAMIFLQENTLLKSELTFQHVKPRLLGHWGTCPGLIFVYSHLNYLIRTMNLDMLYVVGPGHGAPAILAALWLEGSLEKFYPHYSRDEKGLHRLISTFSTTGGFPSHINSETPGAIHEGGELGYALAVSFGAVMDNPDLIVTCIVGDGEAESGPTATSWHAIKYIDPKESGAVLPILHLNGFKISERTIFGCMDHKELLTLFSGYGYQVRFVEDNNDIDADLHTSMIWAVNEIQKIQKAARSGKPIMKPRWPMLILRTSKGWTGPKQLHGKFLEGSYHSHQVPLPKAKTDKEQLDLLQNWLSSYKPEELFTSNGDVIDEIKSVIPTEDKKKLGQRIEVYNSYTPPNLPDWKPFCADKGSQESAMKAAGTFINQTFKDNPNSVRLFSPDELESNKLDAVFEYTNRNFQWDEFANARGGRVIEVLSEHMCQGFMQGYTLTGRIGIFPSYESFLGIIHTMMVQYAKFIKMGLETTWHSGVSSVNYIESSTWARQEHNGFSHQNPSFIGAVLKLKPSAARVYLPPDANTFLTTIHHCLKSKNYINLMVGSKQPTPVYLTPKEAESHCRAGASIWKFCSTDDGINPDVVLVGIGVEVMFEVIAAAALLRKLIPELRVCVINVTDLMILDNEGAHPHALSTEAFDGLFTSDRPIHFNYHGYPTELQGLLFGRPRLDRVSVAGYIEEGSTTTPFDMMLVNRVSRFHVAQHAIRGAAKVNEKVRVYQQELNAQLEASMVSTRKYIVENRDDPDGIYDMPQFHSFHKPAESETFWNIAQ

SEQ ID NO: 71 - protein, 780 aa, Aspergillus nidulans
MPGEVIERPNPAPKPSHVPDLVEKLIIPAQKTKLEKSDCDALHKYRRAAAYIAAGHWGTCPGLILVYSHLNYLIKKQNLDMLYVVGPGHGAPGLLASLWLEGSLGKFYPQYTKDKEGLHNLISTFSTSAGLPSHINAETPGAIHEGGELGYALSVSFGAVMDNPDLIVTCVVGDGEAETGPTATSWHAIKYIDPAESGAVLPILHVNGFKISERTIFGCMDNREIVCLFTGYGYQVRIVEDLEDIDNDLHSAMSWAVEEIRNIQKAARSGKPIMKPQWPMIVLRTPKGWSGPKELHGQFIEGSFHSHQVPLPNAKKDDEELQALQKWLSSYKPDELFTESGDVIDEILSIIPSDDKKLGMRPEAYKTHLPPDLPDWRQFCVKKGDQFSAMKAIGSFIDQVFVKNPHTVRLFSPDELESNKLSAALSHTGRNFQWDEFSNAKGGRVIEVLSEHLCQGFMQGYTLTGRTGIFPSYESFLGIIHTMMVQYAKFAKMAKETAWHHDVSSINYIETSTWARQEHNGFSHQNPSFIGAVLKLKPYAARVYLPPDANTFLTTLHHCLKSKNYINLMVGSKQPTPVYLSPEEAESHCRAGASIFKFCSTDGGLRPDVVLVGIGVEVMFEVIKAAAILRERCPELRVRVVNVTDLFILENEGAHPHALKHEAFDNLFTEDRSIHFNYHGYVNELQGLLFGRPRLDRATIKGYKEEGSTTTPFDMMLVNEVSRYHVAKAAVTGGARFNEKVKLRHQELCSEFDHNIAETRKYIMNNHQDPEDTYNMPSFN

SEQ ID NO: 72 - protein, 806 aa, Aspergillus clavatus
MPGEVIDRPNPQALPSHIPDVVEQLQVKLDLTSLEQPACDALFKFRRAASYIAAAMIFLQDNVLLKRSLTHDDIKPRLLGHWGTCPGLILVYSHLNYLITKRNLNMLYVVGPGHGAPAILASLWLEGSLEKFYPEYSRNAEGLANLISTFSTTAGLPSHINAETPGAIHEGGELGYALAVSFGAVMDNPNLIVPCIVGDGEAETGPTAASWHGIKYIDPAESGAVLPILHLNGFKISERTIFGCMDDKELTSLFTGYGYQVRIVQDLNDIDTDLHRSMTWALEEIHKIQQAARSGKPIMKPRWPMLILRTPKGWSGPKEIHGQFIEGSFHSHQVPLPNAKKDKEELQALEKWLSSYGPRELFTENGDVIDEIKSIIPTDDAMKLGQRVEAYKAYLPPKLPDWRKFAVDKGSQQSPMKTTGKLIDEVFQLNPHTVRLFSPDELESNKLSAALDHTGRNFQWDQFSSAKGGRVIEVLSEHMCQGFIQGYTLTGRTGIFPSYESFLGIVHTMMVQYSKFIKMAQETTWHRSIASINYIETSTWTRQEHNGFSHQNPSFIGAVLKLKPTAARVYLPPDANTFLITLHHCLKSTNYINLMVSSKQPTPVYLTPEEAESHCRAGASVWKFCSTDDGLDPDVVLVGIGVEVMFEVIAAAAILRKRCPELRVRVVNVTDLMILENEGAHPHALTTDAFDSLFTSDRPIHFNYHGYPTELQGLLFGRPRLERATIGGYMEEGSTTTPFDMMLVNRVSRFHVAQAAICGASKRNERVQIRQQELYTELDHNMVETKKYILKNRKDPDDMYDMPKFE

SEQ ID NO: 73 - protein, 788 aa, Leuconostoc mesenteroides
MNIDSTDYLNNLDAYWRATNYLSVGQLYLLDNPLLKEKLTAEQVKIHPIGHWGTIPSQNFIYAHLNRAINKFNLNMFYIEGPGHGGQVMISNAYLDGSYTEAFPEITQDEAGMQKMFKRFSFPGGVASHADPKVPGSIHEGGALGYSILHGAGAVLDNPDLIAAVVVGDGEAETAPLATSWHVNKFLNPKNDGTVLPILNLNGFKIANPTVLSRESDETLTDYFHSLGWHPYFVSSFDKPIMQVHEEMAKTMDTVFTEIKDIREKAVQQTNEEITRPLWPMIVLRSPKGWTGPKTWDDKPIENSFRAHQIPIPADQNHPEYIPQLVDWLQSYKPDELFDENGQLTQSIQEVLPKKELRMANNSVTNAGIIKPLILPDIDNYLVENNQPDNNLAQDAILLGDYLRDIIKLNPTNFRGFGPDETASNRFQDIFETTNRQWLLPIKEPNDQFMAPEGRIIDSMLSEHYDEGMLEAYTLTGRHGFFASYEVFIREVDDMIVQHFKWLNHSHDVSWRKDVPALNIIADSTVFQQDHNGYSHQDPGVTTMLYEKQPDFIREFFPADANSLVATFEHAAQATQQINYIVASKHPRLQWFSPTEAKQLVTQGLRVIDWASTDKGEKPDIIITSAGSEPTTESLAAIQILHEHIPSLKIRYINVLDLFKLRADASYGLSDDEFDAYFTTDTPVLFAFHGYEPMIESIFFKRHNHHLAVHGYREVGDITTPFDMRVLNKIDRFNLVKAAINLLPENIRTKQAALVQEMTDKLDLHVAYTRSKGTDLPEVEDWRWRPLK

SEQ ID NO: 74 - protein, 794 aa, Oenococcus oeni
MTINYDSKDYLKYVDAYWRAANYLSVGQLFLRNNPLLKDELQSKDVKIKPIGHWGTVAPQNFIYAHLNRAILKYDLNMFYIEGSGHGGQVMVSNSYLDGSYTETYPKVTQDIQGMQRLFKQFSFPGGIASHAAPETPGSIHEGGELGYSISHGVGAILDNPDVIAAVEIGDGESETGPLAASWFSDKFINPIHDGAVLPIVQINGFKISNPTILSRMSDRDLTNYYHGMGWEPLFVETDGSDNFKVHAEMADAVDKAIEKIKAIQKNARNNNDDSLPIWPMIVLRAPKGWTGPKKDLDGNPIENSFRAHQVPIPVDANHLEHKDMLIDWMKSYKPEELFNEDGSLKEIVKVNQPKGQRRMAMNPITNGGIKPRTLNMPDMERFAFPKNSLKNNKKPGMDLQVVSTFIAEIIKKNPINFRQFGPDETMSNRLWDEFKVTNRQWMQAVHEPNDQYMAHSGRILDAQLSEHQAEGWMEGYVLTGRHAFFASYEAFTRVIDSMLTQYYKWLRKAVEQDWRHDYPSLNVINASHAFQQDHNGYTHQDPGMLTHMAEKGHEFVNEFLPADANSLLAVMNKSLQVRNKINIIVASKHPRTQWFTIDEAKELVDNGLGIIPWASNDDGVEPDVVFAAGGTEATMESLAAISLLHESFPELKFRFINVIDLLKLRKKGDNDDYRGLSDLEFDHYFTREKPVVFSFHGFESLARDLFYDRHNHNVIFHGYRENGDITTPFDMRVLNHLDRFHLAKDAINATKYADVAGQFDQRMDDMLAKHTAYICDQGTDLPEVTSWQWQDIK

SEQ ID NO: 75 - protein, 675 aa, Salmonella enterica
MTVKEHKVVHEAQNVEALHAPEHFYKSQPGPSYIKDMKQYKEMYKQSVEDPENFFGEKARELLDWDRPFTRSKYGSLENGDVTWFLNGELNAAYNCVDRHAFANPDKPALIYEADEEADNRMITFSELLRQVSRVAGVLQSWGVKKGDTVAVYLPMIPEAVVAMLAIARLGAIHSVVFAGFSAGSLKDRVVDAGCKVVITCDEGKRGGKTVHTKKIVDEGLNGISLVSHILVFQRTGSEGIPMTAGRDYWWHEETAKQRSYLPPVPCNSEDPLFLLYTSGSTGSPKGVVHSTAGYLLGAAMTTRYVFDIHPEDVLFTAGDVGWITGHTYALYGPLVLGTASIIFESTPAYPDYGRYWRIIQRHKATHFYVAPTALRLIKRVGEAEIPKYDISSLRVLGSVGEPISPELWEWYYEKVGNKNCVICDTMWQTESGSHLIAPQAGAVPTKPGSATVPFFGVDACIIDPVTGIELQGNDVEGVLAVKSSWPSMARSVWQNHHRYVDTYLKPYPGYYFTGDGAGRDHDGYYWIRGRVDDVVNVSGHRLSTAEIEASLTNHDNVSESAVVGIADELTGQSVIAFVSLKDGSSRESSAVVAMRRELVLQVRGEIGPFAAPKCVILVKDLPKTRSGKIMRRVLRKVASNEADQLGDLSTMANSEVVPSIIAAVDEQFFAEKKK

SEQ ID NO: 76 - protein, 675 aa, Zygosaccharomyces bailii; identical to SEQ ID NO: 75

SEQ ID NO: 77 - protein, 590 aa, Acetobacter aceti
MLPWTTYEAMYDAALNQPEQFWLAAAQRVTWKQAPVTACRTRSDGWHDWFPDATLNTCHNAVDRHVENGRGGQAALIWHSCATRERQVITYRELQSRVAGFAGGLRSLGVEKGERVLIAMPTMIETVIAMLACARIGAVHVVVFAGYAGPELARRIDDAAPKVIIIASCSFQGQTPVPSVPALNEALAAATHCPQACVIVQREACPVSLLPVRDHDFHTLEQSAPAEPLMLRSEDPLYILHTSGTTGNAKGIVRDNGGHAVALALSMDLIYGCKPGDTFFTTSDLGWVVGHSYGVYAPLISGCTSVIVEGGASASAIRMLCHEHAVKCLFTTPTQMRLMRQESRHLSGAILPALARIFVAGEYADPTLLEWTRSYFRKPVVNHWWQTETGWSITAHFFGLPEREPVSLMNDIGRPAPGFCPAIVPSIADEQYGEIVLSLPLPPGCLAGMWKDGAIRLPSTYLDEIGRYYRTFDEGMIEANRAVHMLGRSDDVIKVAGRRISGVQIEKIIATHPAVHTCAVVAIPDELRGQRPVAYVVVDPEASCEPSSEEIVVLVNEVLGRWVGLKEVRFIRHLPTTVSGKITRKRLLVS

SEQ ID NO: 78 - protein, 713 aa, Saccharomyces cerevisiae
MSPSAVQSSKLEEQSSEIDKLKAKMSQSASTAQQKKEHEYEHLTSVKIVPQRPISDRLQPAIATHYSPHLDGLQDYQRLHKESIEDPAKFFGSKATQFLNWSKPFDKVFIPDPKTGRPSFQNNAWFLNGQLNACYNCVDRHALKTPNKKAIIFEGDEPGQGYSITYKELLEEVCQVAQVLTYSMGVRKGDTVAVYMPMVPEAIITLLAISRIGAIHSVVFAGFSSNSLRDRINDGDSKVVITTDESNRGGKVIETKRIVDDALRETPGVRHVLVYRKTNNPSVAFHAPRDLDWATEKKKYKTYYPCTPVDSEDPLFLLYTSGSTGAPKGVQHSTAGYLLGALLTMRYTFDTHQEDVFFTAGDIGWITGHTYVVYGPLLYGCATLVFEGTPAYPNYSRYWDIIDEHKVTQFYVAPTALRLLKRAGDSYIENHSLKSLRCLGSVGEPIAAEVWEWYSEKIGKNEIPIVDTYWQTESGSHLVTPLAGGVTPMKPGSASFPFFGIDAVVLDPNTGEELNTSHAEGVLAVKAAWPSFARTIWKNHDRYLDTYLNPYPGYYFTGDGAAKDKDGYIWILGRVDDVVNVSGHRLSTAEIEAAIIEDPIVAECAVVGFNDDLTGQAVAAFVVLKNKSSWSTATDDELQDIKKHLVFTVRKDIGPFAAPKLIILVDDLPKTRSGKIMRRILRKILAGESDQLGDVSTLSNPGIVRHLIDSVKL

SEQ ID NO: 79 - protein, 683 aa, Saccharomyces cerevisiae
MTIKEHKVVHEAHNVKALKAPQHFYNSQPGKGYVTDMQHYQEMYQQSINEPEKFFDKMAKEYLHWDAPYTKVQSGSLNNGDVAWFLNGKLNASYNCVDRHAFANPDKPALIYEADDESDNKIITFGELLRKVSQIAGVLKSWGVKKGDTVAIYLPMIPEAVIAMLAVARIGAIHSVVFAGFSAGSLKDRVVDANSKVVITCDEGKRGGKIINTKKIVDEGLNGVDLVSRILVFQRTGTEGIPMKAGRDYWWHEEAAKQRTYLPPVSCDAEDPLFLLYTSGSTGSPKGVVHTTGGYLLGAALTTRYVFDIHPEDVLFTAGDVGWITGHTYALYGPLTLGTASIIFESTPAYPDYGRYWRIIQRHKATHFYVAPTALRLIKRVGEAEIAKYDTSSLRVLGSVGEPISPDLWEWYHEKVGNKNCVICDTMWQTESGSHLIAPLAGAVPTKPGSATVPFFGINACIIDPVTGVELEGNDVEGVLAVKSPWPSMARSVWNHHDRYMDTYLKPYPGHYFTGDGAGRDHDGYYWIRGRVDDVVNVSGHRLSTSEIEASISNHENVSEAAVVGIPDELTGQTVVAYVSLKDGYLQNNATEGDAEHITPDNLRRELILQVRGEIGPFASPKTIILVRDLPRTRSGKIMRRVLRKVASNEAEQLGDLTTLANPEVVPAIISAVENQFFSQKKK

SEQ ID NO: 80 - DNA, 1134 bp, synthetic construct
  source 1..1134: mol_type = other DNA
atgagtgaagatctgatgtttgtctcaccagataagtacattcaaggtgctggtttattggaacacagtgcaagttatatcaatgcgctaggttcacatccattgattatggctgatgcgctaatttggaaaattgttggtgaagattttgccacgtacttgtcccagcacgatttcgacgtgactcgagtcacgttcaatggggaagcgagtgttcaagaaatcgcccgtattactcaaatcgctaaggataaccataatgatatcattatcgggttagggggaggtaagaccttggatacgtccaaaggcattgcggaagaagttggtgccaagctcgtcattgcaccgaccgccgcgtcagctgacgccccaaccgcaggtttaagcgttttatatacggaagaaggcagttttgatcagtataagttctaccaacgtcattcagatctagttctcatggatacgcaagtcatcgccaatgcaccagtccgaacgctgatttcaggcatcgcggatgcgatggcaaccaatatcgaagccaaggctacggctcaaagtcacgggactacgatgggtaatggggctgcgacgtttactgggcgggccattgcagctgaatgcgaacgtgttatttggaaatacgcctatgaagctgttgaaagcaatgtccaacacattgttaccccggccttagacgccattgttgaagcaaacacgttgttatccggtctagggtttgaaaatggtgggttggcagccgcacatgcaattcacaacggttttacggccgtcaagggtgatgtgcaccatctgacgcacggagaaaaagtggcttttggcacgatggttgaattagtcttgcagggaaccagtcacgaagaattgaatcgatacattgaattttatctgactttgggcttgccgattacgctagaagccacgcacttagatcagttatcagacgaagaaatgttacaaattggcaagatggcaacgatgccggatgaaacgatgaaaaacatgccatttaaggtcactccgcgggaagtggttaacgctatgaaagcagcttccacgttcgcgcatacgtacgaagctacgcataccattaaacctatcctaccagcttaa

SEQ ID NO: 81 - DNA, 999 bp, synthetic construct
  source 1..999: mol_type = other DNA
atgaagaagattattaatgatccaaagaatgtcgtcgaagaaatggtcgatggtttagtccggagttatccacaatatttaacgaagttaccagatacggaagccatggtccggagtgatcaagccagtatgaagggtaaggtcggtttagtcagtggtggtggtagtggtcatgaaccagcccatgccggttttgtcggtcaaggtatgttaagtgccgccgtcgccggtcaagtctttacgagtccaacgccagatcaaatttatgccgccattaaggccgtcgatcaaggtaagggtgtctttttagtcattaagaattatagtggtgatgtcatgaattttgatatggccaaggatatggccgaattagatgatattcaagtcaagagtattgtcgtcgatgatgatattgccgtcaaggatagtttatatacgcaaggtcggcggggtgtcgccggtacggtcttaatgcataagattttaggtgccgccgccgatcaaggtgccagtttagatgaattagaaacgttagccaatcaagtcatgccacaattaaagacgattgccgtcgccttaagtgccgccacggtcccagaagtcggtaagccaggttttgaattagccgatgatgaaattgaatttggtgtcggtattcatagtgaaccaggttatcggcgggaaaagattaagccaagtaaggccttagtccaagaattattaggtaagttagatgatgaattaaagttacaagccgatcatcaatatgccgtcttagtcaatggtatgggtgccacgccattaatggatcaatatgtcttttgtcatgatttattagatgaattagccgcccgggatattgaaccagcctttatgaaggtcggtaattatatgacgagtattgatatggccggtattagtttaacgttattagatatgacgacgacgaattggttagattatttacaatatccagtcaagacgattgcctggagttaa

SEQ ID NO: 82 - DNA, 582 bp, synthetic construct
  source 1..582: mol_type = other DNA
atgttaacggtcgaaaatacgacggcctggttaaatcaatttgccgcccaaattgaacaacatcaaagttatttaagtgaattagatacgccaattggtgatggtgatcatggtggtaatatggcccggggtttagccgccgtcaagacggccttagccacgcaaccagccgatttaacgaagttatttcaagccacggccatggccttaattagtaaggtcggtggtgccagtggtccattatatggtacggcctttttagaaatggccaagcaaagtaagacggatacggatttaggtacgttattagccgccgccttagccggtattgaaaagcggggtggtgccacgacgggtgataagacgttaattgatgtctgggccccagtcgtcgccgccgtccaagccggtaagttaacgcaaacggtcattgatgatgccgtcgccagtacgaagccaatggtcgccaagaagggtcgggccagttatttaggtgaacggagtgccggtcatttagatccaggtgccgtcagtagtggttatttatttacgacgttattagccacggaaggtattttatga

SEQ ID NO: 83 - DNA, 369 bp, synthetic construct
  source 1..369: mol_type = other DNA
atgagtttaggtattgtcgtcgtcagtcatgtcccagaaattgccgccggtgtccaacggttattagcccaagtcgccaaggatgtcagtattacgattgccggtggtacggatgatgatcaagtcggtacgagtatggccaagattacggaagcctttgaagccaatacggccgatgaattattagccttttatgatttaggtagtgccaagatgaatttagaaatggccattgaaatgagtacgaagccagtccatttatatgatacggccttagtcgaaagtgcctatacggccgccagtttattacaagccgatgtcccattagccgatgtcgaagcccaattagccccattaaagattaagtag

SEQ ID NO: 84 - protein, 332 aa, Lacticaseibacillus paracasei
MKKIINDANLVLEDMLKGMVAAHPEYIKKLENADVLVRVDSPIDGKVALVSGGGSGHEPAHGGYVGKGMLDAAVAGAVFTSPTPDQVYEAIKAVDLGKGVLLVIKNYTGDVMNFEMAKDMAEMEGINVKTVVVNDDVAVENSTYTAGRRGIAGTVFIHKIAGAKAETGASLEEVTRVAEKVISNVRSMGMAISSCIVPAAGKPNFTLGENEMEIGMGIHGEPGTHREEIKTADEITEHLMSKILEDIKLSSGEEVAVMVNGLSSTPLMELYIVNKKVNEILEEKGVKIHKTFVGEFMTSLEMAGFSITVLKLDSELKELLDAPANTPAFKVI

SEQ ID NO: 85 - protein, 209 aa, Lacticaseibacillus paracasei
MSIQGKKIIEILEKISEKIDENKAYLSELDAAIGDGDHGLNMNKGFKAVVEKIKDDDGNDIGGILKKSGMALVSNVGGASGPLYGTAFMKAAVSVNGKSEVDINDFANMLDEALEGIKMRGKGQADDKTMIDAIEPALKSIKEGIDSGLEAKEILKVAKEAAYKGVEHTKEIIAKKGRASYLGERSLGHQDAGATSSAIILETIYEALN

SEQ ID NO: 86 - protein, 129 aa, Lacticaseibacillus paracasei
MVGIVIISHSKNIADGVKELASQMAPNVAIGVAGGTADGRVGTDMEKISAAIEDVYSEDGVIIIFDLGSAFMNAEMAIEFLDEKMKEKIKIVDCPIVEGAVTAAVESSIGKNIEEIEEALKPMNLGKMP

SEQ ID NO: 87 - DNA, 1134 bp, synthetic construct
  source 1..1134: mol_type = other DNA
atgtcagaagatttgatgttcgtttcaccagataagtatatccaaggcgctggcttgttggaacattcagcttcatatatcaacgctttgggctcacatccattgatcatggctgatgctttgatctggaagatcgttggcgaagatttcgctacctatttgtcacaacatgatttcgatgttacccgtgttaccttcaacggcgaagcttcagttcaagaaatcgctcgtatcacccaaatcgctaaggataaccataacgatatcatcatcggcttgggcggcggcaagaccttggatacctcaaagggcatcgctgaagaagttggcgctaagttggttatcgctccaaccgctgcttcagctgatgctccaaccgctggcttgtcagttttgtataccgaagaaggctcattcgatcaatataagttctatcaacgtcattcagatttggttttgatggatacccaagttatcgctaacgctccagttcgtaccttgatctcaggcatcgctgatgctatggctaccaacatcgaagctaaggctaccgctcaatcacatggcaccaccatgggcaacggcgctgctaccttcaccggccgtgctatcgctgctgaatgcgaacgtgttatctggaagtatgcttatgaagctgttgaatcaaacgttcaacatatcgttaccccagctttggatgctatcgttgaagctaacaccttgttgtcaggcttgggcttcgaaaacggcggcttggctgctgctcatgctatccataacggcttcaccgctgttaagggcgatgttcatcatttgacccatggcgaaaaggttgctttcggcaccatggttgaattggttttgcaaggcacctcacatgaagaattgaaccgttatatcgaattctatttgaccttgggcttgccaatcaccttggaagctacccatttggatcaattgtcagatgaagaaatgttgcaaatcggcaagatggctaccatgccagatgaaaccatgaagaacatgccattcaaggttaccccacgtgaagttgttaacgctatgaaggctgcttcaaccttcgctcatacctatgaagctacccataccatcaagccaatcttgccagcttaa

SEQ ID NO: 88 - DNA, 1020 bp, synthetic construct
  source 1..1020: mol_type = other DNA
atgaagaagatcatcaacgctccaggcgatatcgttccagaaatgatcgctggcttggctgcttcatatccacaatatttgaagcaaatcccagataccaccgctgttgttcgtaacgatgctgctttcaagcaacataagaaggttggcgttatctcaggcggcggctcaggccatgaaccattgcatgctggctatgttggccaaggcatgttggatgctgctgttgctggccaagttttcacctcaccaaccccagatcaaatctatgctgctatcaaggaagttgatcaaggcgctggcgttttgatgatcgttaagaactattcaggcgatgttatgaacttcgatatggctaaggatttggcttcagttgatgatatcaccgttgaatcagttgttgttgatgatgatgttgctgttaaggattcattgtatacccaaggccgtcgtggcgttgctggcaccatcttcgctgaaaagatcatcggcgctgctgctgaagctggcttgtcattggatgatttgaagaagttgggcgatgctgttgttaagaacaccaagtcattcgctgttgctttgcatgctgctaccgttccagaagttggcaagccaggcttcgatttgaagccagatgaaatcgaattcggcgttggcatccataacgaaccaggcaccggccaagaaaagttgccaacctcaaagcaattggctaagcaattgatctcaaagttgaccgctgaattcaaggatccacaagctcataagtatgctgttttggttaacggcatgggcgctaccccattgtcagaacaatatatcttcatgggcgatgttttgaacgaattgaagaccgaaaagttgccagtttcattcaccaaggctggcaactatgttacctcattggatatggctggcatctcattgaccatgttggctttggatgatccacaatggttgacctatttgaacgcttcagctcataccgttggctggggcgataccgatgttaagcgttga

SEQ ID NO: 89 - DNA, 582 bp, synthetic construct
  source 1..582: mol_type = other DNA
atgttgaacgctgataccttgaccaagtggatgaccaacttcgaaacccgtatcaccaaggaaaaggatcatttgaccgaattggatcgtgttatcggcgattcagatcatggcaacaacatggaacgtggcgttgaagctatcaaggctgctttcgaaaagaccccaccaaccaccgatttggctcaagatttcaagaccatcgctatggctatgttgtcaaaggttggcggcgcttcaggcccattgtatggcaccgctttcttggaaatggctaagttggctaagaccgataccgatttcggcgatttggttgatgctgctgctaagggcatcgctaagcgtggccaagctaagaccggcgataagaccatgttggatgtttgggctccagctgctgatttgatcaagcaaggcaagttggatgatgctgctatcgataagttggttgcttcaaccgaaccaatgatcgctaagcgtggccgtgcttcatatttgggcgaaaagtcagttggccatatcgatccaggcgctgcttcaaccggcgaattgttgaaggcttatttggatgctaccgcttga

SEQ ID NO: 90 - DNA, 369 bp, synthetic construct
  source 1..369: mol_type = other DNA
atggctttgggcatcttgttggtttcacatgttccagctatcgctaacggcgttgaaaccttgatcaagcaagttgctaaggatgttccaatcaccaccgctggcggcacctcagatggcaagatcggcaccgatttggatcatatccaaaaggctatcggcgataacccagcttcagaattgttggttttctatgatttgggctcagctaagatgaacttggatatcgctgaagaagtttcagataagcaaatgcatgtttatgatgttgctttcgttgaaggcgcttatgctgctgctgctttgttggaagctaacgttaccttggataagatcgaagctcaattgaagccattgaaggttaagtaa

(19) United States
(12) Patent Application Publication
(10) Pub. No.: US 2024/0191263 A1
(43) Pub. Date: Jun. 13, 2024

(54) BACTERIAL AND YEAST COMBINATIONS FOR REDUCING GREENHOUSE GAS PRODUCTION DURING FERMENTATION OF BIOMASS COMPRISING HEXOSES

(71) Applicants: DANSTAR FERMENT AG, Zug (CH); LALLEMAND HUNGARY LIQUIDITY MANAGEMENT LLC, Budapest (HU)

(72) Inventors: Jeffery R. Broadbent, Lebanon, NH (US); James L. Steele, Lebanon, NH (US); Brooks Henningsen, Lebanon, NH (US); Ekkarat Phrommao, Lebanon, NH (US); Fernanda Cristina Firmino, Milwaukee, WI (US)

(21) Appl. No.: 18/537,421

(22) Filed: Dec. 12, 2023

Related U.S. Application Data
(60) Provisional application No. 63/387,060, filed on Dec. 12, 2022.

(51) Int. Cl. C12P 7/14; C12N 9/04; C12N 9/12; C12N 9/88; C12N 15/52

(52) U.S. Cl. CPC C12P 7/14; C12N 9/0006; C12N 9/1205; C12N 9/88; C12N 15/52; C12Y 101/01001; C12Y 101/01006; C12Y 207/01121; C12Y 401/01001; C12Y 401/02009

(57) ABSTRACT

The present disclosure concerns a symbiotic combination of a bacterial host cell and a yeast host cell selected or engineered to utilize glycerol to reduce greenhouse gases during the production of ethanol from a biomass comprising hexoses.

### CROSS-REFERENCE TO RELATED APPLICATION AND DOCUMENT

The present application claims priority from U.S. provisional patent application 63/387,060 filed on Dec. 12, 2022, herewith incorporated in its entirety. The present application also includes a sequence listing in electronic format which is also incorporated in its entirety.

### REFERENCE TO AN ELECTRONIC SEQUENCE LISTING

The contents of the electronic sequence listing (580127_443_SeqListing.xml; Size: 145,723 bytes; and Date of Creation: Nov. 27, 2023) is herein incorporated by reference in its entirety.

### TECHNOLOGICAL FIELD

The present disclosure concerns a combination of a bacterial host cell and a yeast host cell for reducing greenhouse gas production during the bioconversion of a biomass into ethanol.

### BACKGROUND

The yeast Saccharomyces cerevisiae is utilized as the primary biocatalyst in commercial bioethanol production. In its native (non-genetically modified) form, the yeast is able to convert, during glycolysis, each molecule of hexose sugars (such as glucose) into two molecules of each of ethanol and carbon dioxide (CO₂) as follows:

Glucose+2 Pi+2ADP→2 Ethanol+2ATP+2CO₂ (Reaction A)

It would be highly desirable to be provided with means of decreasing CO₂ production during fermentation processes in which a yeast is used as a fermentation organism to produce ethanol.

### BRIEF SUMMARY

The present disclosure provides using a bacterial host cell capable of utilizing glycerol in combination with a yeast host cell to produce ethanol from a biomass comprising hexoses. The combination of yeast host cell and bacterial host cell of the present disclosure reduces in the accumulation of greenhouse gases, like CO₂, during the fermentation process while maintaining the ethanol yield.

According to a first aspect, the present disclosure comprises a combination for making ethanol from a biomass comprising hexoses, the combination comprising a yeast host cell and a bacterial host cell. The bacterial host cell has: a first metabolic pathway comprising one or more first polypeptides for converting acetate into ethanol; a second metabolic pathway comprising one or more second polypeptides for the conversion of glycerol into dihydroxyacetone phosphate; a third metabolic pathway comprising one or more third heterologous polypeptides for converting pyruvate into ethanol. The yeast host cell has: a fourth metabolic pathway comprising one or more fourth polypeptides for producing glycerol; and a fifth metabolic pathway comprising one or more fifth polypeptides for generating acetate. In an embodiment, the hexoses comprise glucose. In another embodiment, the biomass comprises or is derived from corn. In still another embodiment, the one or more first polypeptides comprises: one or more native or heterologous enzymes for converting acetate into acetyl-CoA; and/or one or more native or heterologous enzymes for converting acetyl-CoA into acetaldehyde, and optionally acetaldehyde into ethanol. In still another embodiment, the one or more native or heterologous enzymes for converting acetate into acetyl-CoA comprise: a polypeptide having an acetate kinase (ACK) activity, a polypeptide having phosphotransacetylase (PTA) activity; and/or a polypeptide having acetyl-coA synthetase activity. In an embodiment, the one or more native or heterologous enzymes for converting acetate into acetyl-CoA are native. In still another embodiment, the one or more native or heterologous enzymes for converting acetyl-CoA into acetaldehyde, and optionally acetaldehyde into ethanol, comprise: a polypeptide having an acetaldehyde dehydrogenase (AADH) activity, a polypeptide having an alcohol dehydrogenase activity, and/or a polypeptide having a bifunctional acetaldehyde/alcohol dehydrogenase (ADHE) activity. In still another embodiment, the one or more enzymes for converting acetyl-CoA into acetaldehyde, and optionally acetaldehyde into ethanol, are native. In some embodiments, the second metabolic pathway is for the conversion of glycerol into dihydroxyacetone phosphate (and is some additional embodiments, for the dehydrogenation of glycerol). In yet another embodiment, the one or more second polypeptides comprise: a native or heterologous polypeptide having glycerol dehydrogenase (GLDA) activity or a combination of the native and the heterologous polypeptide having GLDA activity, a native or heterologous polypeptide having an ATP-dependent dihydroxyacetone kinase (DAK) activity, and/or a native or heterologous polypeptide having a PEP-dependent dihydroxyacetone kinase (DHAKLM) activity or a combination of the native and the heterologous polypeptide having DHAKLM activity. In some embodiments, the one or more second polypeptides are native. In other embodiments, the one or more second polypeptides are heterologous. In some embodiments, the one of or more third heterologous polypeptides comprise: a native or heterologous polypeptide having pyruvate decarboxylase (PDC) activity, and/or a native or heterologous polypeptide having alcohol dehydrogenase (ADH) activity. In some embodiments, the polypeptide having PDC activity is heterologous. Alternatively or in combination, the polypeptide having ADH activity is heterologous. In still another embodiment, the bacterial host cell is a lactic acid bacterium. In additional embodiments, the bacterial host cell is from Lactiplantibacillus sp., and in yet further embodiments, the bacterial host cell is from Lactiplantibacillus pentosus or from Lactiplantibacillus plantarum. In further embodiments, the bacterial host cell is from Lacticaseibacillus sp., and in yet further embodiments, the bacterial host cell is from Lacticaseibacillus paracasei. In some embodiments, the bacterial host cell has a decreased lactate dehydrogenase activity and optionally at least one inactivated native gene coding for a lactate dehydrogenase. In additional embodiments, the bacterial host cell includes at least one genetic modification for reducing carbon catabolite repression, when compared to a control bacterial host cell lacking the at least one genetic modification. For example, the at least one genetic modification comprises a genetic modification for decreasing the expression or inactivating a gene encoding a phosphoenolpyruvate-dependent phosphotransferase (PTS) transporter (such as, for example, a mannose PTS transporter). In some embodiments, the bacterial host cell includes at least one genetic modification for decreasing the expression or inactivating at least one gene encoding a polypeptide involved in a glycolytic flux. In some additional embodiments, the at least one gene encoding a polypeptide involved in a glycolytic flux comprises a gene encoding a glucose permease, a maltose PTS transporter, a maltose/maltodextrin transporter, a kinase (such as, for example, a glucokinase) or a transcription factor (such as, for example, REX). In some embodiments, the one or more fourth polypeptides comprise: a native or heterologous polypeptide having glycerol-3-phosphate dehydrogenase activity, and/or a native or heterologous polypeptide having glycerol-3-phosphate phosphatase activity. In some embodiments, the native or heterologous polypeptide having glycerol-3-phosphate dehydrogenase activity is a polypeptide having NAD-dependent glycerol-3-phosphate dehydrogenase activity. In yet additional embodiments, the native or heterologous polypeptide having NAD-dependent glycerol-3-phosphate dehydrogenase activity comprises GPD1 and/or GPD2. In still other embodiments, the native or heterologous polypeptide having glycerol-3-phosphate phosphatase activity comprises GPP1 and/GPP2. In yet additional embodiments, the yeast host cell has the native fourth metabolic pathway. In some additional embodiments, the one or more fifth polypeptides for generating acetate comprises one or more native or heterologous polypeptides having phosphoketolase activity, wherein the phosphoketolase has single specificity or dual specificity and optionally exhibits a phosphatase activity. In some embodiments, the one or more polypeptides having phosphoketolase activity are heterologous. In additional embodiments, the yeast host cell is from Saccharomyces sp., and yet in further embodiments, the yeast host cell is from Saccharomyces cerevisiae.

According to a second aspect, the present disclosure provides a bacterial host cell for making ethanol from a biomass comprising hexoses, the bacterial host cell comprising: a first metabolic pathway comprising one or more first polypeptides for converting acetate into ethanol; a second metabolic pathway comprising one or more second polypeptides for the converting glycerol into dihydroxyacetone phosphate; and a third metabolic pathway comprising one or more third heterologous polypeptides for converting pyruvate into ethanol. In some embodiments, the biomass is the one described herein. In still another embodiment, the one or more first polypeptides are those described herein. In yet another embodiment, the one or more second polypeptides are those described herein. In yet another embodiment, the one or more third heterologous polypeptides are those described herein. In some embodiments, the bacterial host cell is the one described herein.

According to a third aspect, the present disclosure provides a composition comprising (i) the combination described herein or the bacterial host cell described herein and (ii) a biomass comprising hexoses.

According to a fourth aspect, the present disclosure provides a process for converting a biomass comprising hexoses into ethanol. The process comprises contacting the biomass with (i) the combination described herein or (ii) the bacterial host cell described herein and a fermenting yeast under a condition to allow the conversion of at least a part of the biomass into ethanol. In an embodiment, the fermenting yeast is the yeast host cell described herein. In still another embodiment, the process comprises contacting the biomass first with the fermenting yeast.

According to a fifth aspect, the present disclosure provides a process for reducing the emission of CO₂ during the conversion of a biomass comprising hexoses into ethanol. The process comprises contacting the biomass with (i) the combination described herein or (ii) the bacterial host cell described herein and a fermenting yeast under a condition to allow the conversion of at least a part of the biomass into ethanol, wherein the reduction in the emission of CO₂ is observed when comparing a process performed in the absence of the bacterial host cell. In an embodiment, the fermenting yeast is the yeast host cell described herein. In still another embodiment, the process comprises contacting the biomass first with the fermenting yeast.

According to a sixth aspect, the present disclosure provides a process for improving the fermentation yield during the conversion of a biomass comprising hexoses into ethanol. The process comprises contacting the biomass with (i) the combination described herein or (ii) the bacterial host cell described herein and a fermenting yeast under a condition to allow the conversion of at least a part of the biomass into ethanol, wherein the improvement in the fermentation yield is observed compared to a control process performed in the absence of the bacterial host cell. In an embodiment, the fermenting yeast is the yeast host cell described herein. In still another embodiment, the process comprises contacting the biomass first with the fermenting yeast.

### BRIEF DESCRIPTION OF THE DRAWINGS

Having thus generally described the nature of the invention, reference will now be made to the accompanying drawings, showing by way of illustration, a preferred embodiment thereof, and in which:

FIG. 1 provides an embodiment of the combination for making ethanol from a biomass comprising hexoses.

### DETAILED DESCRIPTION

The present disclosure provides a yeast/bacteria consortium that can reduce the overall greenhouse gas (including CO₂) production and provide the same or a higher ethanol yield (when compared to a corresponding native or recombinant yeast) during the fermentation of a biomass. The yeast/bacteria consortium (also referred herein as a “combination comprising a yeast host cell and a bacterial host cell”) allows the efficient utilization of glycerol by the bacterial host cell while maintaining its redox balance. The latter is possible by increasing the amount of acetate/acetyl phosphate available to the bacterial host cell and allowing the bacterial host cell to convert it to ethanol. The glycerol utilized by the bacterial host cell is produced by the yeast host cell.

In the present disclosure, the combination of the present disclosure is designed for the fermentation of a biomass comprising hexoses (such as in a biomass comprising corn or being derived from corn for example) into ethanol. In the context of the present disclosure, a biomass comprising hexoses refers to a biomass in which the majority of the carbohydrates are hexoses (including, but not limited to glucose). The biomass can include, in some embodiments, pentoses (like xylose and arabinose for example), but the amount of pentoses in the biomass is less than the amount of hexoses in the biomass. In some embodiments, the biomass comprises a minimal amount of glucose of at least about 12.5 mM.

The bacterial host cell of the present disclosure comprises a first metabolic pathway for converting acetate into ethanol, e.g., the bacterial host cell comprises one or more first polypeptides involved in the conversion of acetate into ethanol. The bacterial host cell, is either selected for its native ability to convert acetate into ethanol or is engineered to increase its activity to convert acetate into ethanol. The bacterial host cell of the present disclosure also comprises a second metabolic pathway for converting glycerol into dihydroxyacetone phosphate (which can, in some embodiments, be a metabolic pathway for the dehydrogenation of glycerol). The bacterial host cell is either selected for its native ability to dehydrogenate glycerol or is engineered to increase its activity to dehydrogenate glycerol. The bacterial host cell of the present disclosure also comprises a third metabolic pathway for converting pyruvate into ethanol. The bacterial host cell is engineered to increase its activity to convert pyruvate into ethanol. Because the combination is designed for the fermentation of hexose sugars (such as glucose that is present in a biomass comprising starch for example) in ethanol, the yeast host cell comprises a metabolic pathway for generating acetate. In addition, the yeast also has the ability to produce glycerol.

Under these circumstances, the yeast host cell can convert two molecules of glucose into two molecules each of ethanol and carbon dioxide (CO₂) as well as three molecules of acetate as follows:

2 Glucose+5ADP+5 Pi→2 Ethanol+2CO₂+5ATP+3 Acetate (Reaction B)

The bacterial host cell, because it is capable of utilizing glycerol while maintaining its redox balance (by converting acetate into ethanol), is capable of using both the glycerol and acetate produced by the yeast to generate ethanol as follows:

3 Acetate+6 Glycerol+3 Pi+3ADP→9EtOH+3ATP+6CO₂ (Reaction C)

Under conditions where the glycerol content is not limited, the overall stoichiometry for the combination is as follows:

2 Glucose+6 glycerol+8 Pi+8ADP→11EtOH+8ATP+8CO₂ (Reaction D)

When compared to Reaction A provided above (provided for a conventional yeast fermenting hexose sugars such as glucose), overall reaction D decreases the amount of CO₂ created for each molecule of ethanol produced. Because acetate produced by the yeast can be converted to ethanol by the bacterium, and that conversion of acetate to ethanol does not result in CO₂ production, this configuration of the combination substantially increases the amount of ethanol that can be produced from hexose sugars while reducing the amount of CO₂ that is generated in ethanol bioconversion.

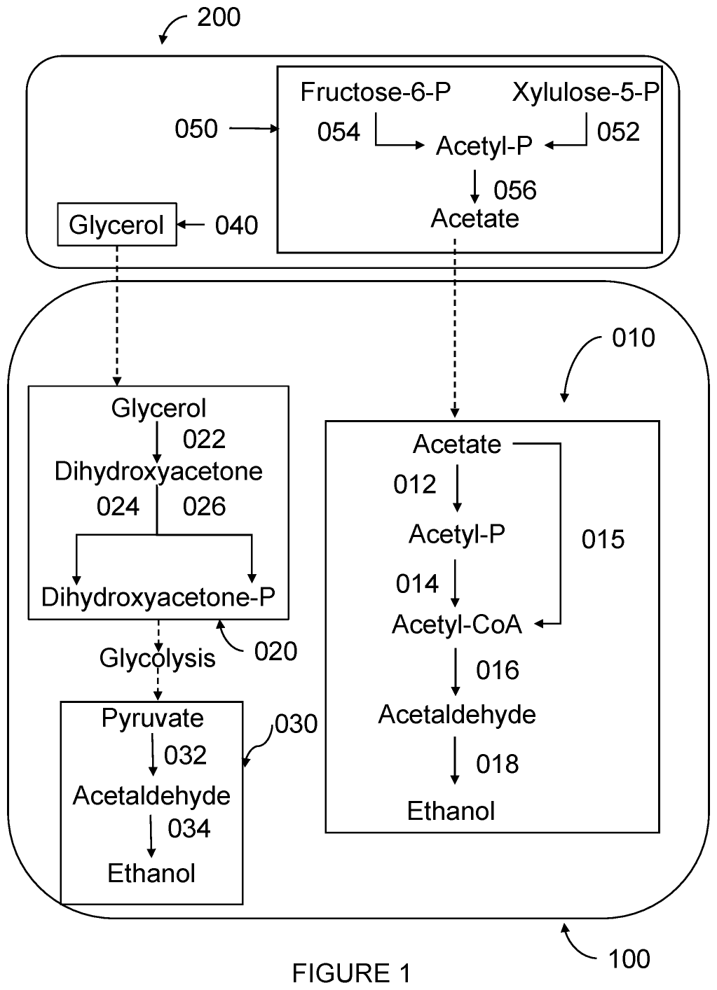

An embodiment of a configuration for the conversion of a biomass comprising hexoses into ethanol is presented in FIG. 1. In FIG. 1, the bacterial host cell 100 and the yeast host cell 200 are provided as components of the combination. The yeast host cell 200 comprises a metabolic pathway 050 for generating acetate from acetyl phosphate, fructose-6-phosphate and/or xylulose-5-phosphate. The metabolic pathway 050 of FIG. 1 can include one or more of the following first enzymes: a polypeptide having xylulose-5-phosphate phosphoketolase activity 052 (to generate acetyl phosphate from xylulose-5-phosphate), a polypeptide having fructose-6-phosphate phosphoketolase activity 054 (to generate acetyl phosphate from fructose-6-phosphate) and/or a polypeptide having phosphatase activity 056 (to generate acetate from acetyl phosphate). In some embodiments, the metabolic pathway 050 of FIG. 1 can include a polypeptide having bifunctional phosphoketolase activity (not shown on the FIGURE) which is capable of converting xylulose-5-phosphate and fructose-6-phosphate in acetyl phosphate.

It is understood that the acetate produced by the yeast host cell 200 in FIG. 1 will become available for metabolism to the bacterial host cell 100. In order to be able to utilize acetate, the bacterial host cell includes metabolic pathway 010 comprising one or more polypeptides for converting acetate into ethanol. Metabolic pathway 010 can comprise a polypeptide having an acetate kinase (ACK) activity 012 (to convert acetate into acetyl phosphate), a polypeptide having a phosphotransacetylase (PTA) activity 014 (to convert acetyl phosphate to acetyl-CoA), a polypeptide having acetyl-CoA synthetase activity 015 (to convert acetate to acetyl-CoA), a polypeptide having an acetylating acetaldehyde dehydrogenase (AADH) activity 016 (to convert acetyl-CoA into acetaldehyde) and/or a polypeptide having an alcohol dehydrogenase activity 018 (to convert acetaldehyde into ethanol). In an embodiment, not shown on FIG. 1, the metabolic pathway 010 can comprise a polypeptide having a bifunctional acetylating acetaldehyde/alcohol dehydrogenase activity (to convert both acetyl-CoA into acetaldehyde and acetaldehyde into ethanol).

The yeast host cell 200 presented on FIG. 1 also includes metabolic pathway 040 comprising one or more polypeptides for producing glycerol. In some embodiments, the one or more polypeptides for producing glycerol can include a polypeptide having glycerol-3-phosphate dehydrogenase activity and/or a polypeptide having glycerol-3-phosphate phosphatase activity (not shown on FIG. 1). In additional embodiment, the yeast host cell can include a reduction in activity or an inactivation in one or more genes encoding one or more polypeptides capable of catabolizing glycerol (such as, for example, a polypeptide having glycerol dehydrogenase activity and/or a polypeptide having dihydroxyacetone kinase activity, not shown on FIG. 1).

It is understood that the glycerol produced by the yeast host cell 200 in FIG. 1 will become available for metabolism to the bacterial host cell 100. In order to be able to utilize glycerol, the bacterial host cell 100 includes a metabolic pathway 020 comprising one or more second polypeptides for the conversion of glycerol into dihydroxyacetone phosphate. In the embodiments shown on FIG. 1, the metabolic pathway 020 is for the dehydrogenation of glycerol. Metabolic pathway 020 can include a polypeptide having glycerol dehydrogenase (GLDA) activity 022 (to convert glycerol into dihydroxyacetone), a polypeptide having an ATP-dependent dihydroxyacetone kinase (DAK) activity 024 (to convert dihydroxyacetone to dihydroxyacetone phosphate) and/or a polypeptide having a phosphoenolpyruvate (PEP)-dependent dihydroxyacetone kinase (DHAKLM) activity 026 (to convert dihydroxyacetone to dihydroxyacetone phosphate). The dihydroxyacetone phosphate produced by the glycerol dehydrogenation pathway, during glycolysis, will ultimately be converted to pyruvate, as shown on FIG. 1. The bacterial host cell 100 presented on FIG. 1 further includes metabolic pathway 030 comprising one or more polypeptides for converting pyruvate into ethanol. Metabolic pathway 030 comprises at least one polypeptide having pyruvate decarboxylase activity 032 (to convert pyruvate to ethanol) and a heterologous polypeptide having alcohol dehydrogenase (ADH) activity 034 (to convert acetaldehyde to ethanol).

### Recombinant Host Cells

The combination of the present disclosure comprises a yeast host cell (which can, in some embodiments be a recombinant yeast host cell) and a recombinant bacterial host cell. These recombinant host cells can be obtained by introducing one or more genetic modifications in a corresponding native (parental) yeast/bacterial host cell. When the genetic modification is aimed at reducing or inhibiting the expression of a specific targeted gene (which is endogenous to the host cell), the genetic modifications can be made in one or both copies of the targeted gene(s).

When the genetic modification is aimed at increasing the expression of a specific targeted gene, the genetic modification can be made in one or multiple genetic locations. In the context of the present disclosure, when a yeast and a bacterial host cell are qualified as being “genetically engineered”, it is understood to mean that they have been manipulated to either add at least one or more heterologous or exogenous nucleic acid residue and/or removed at least one endogenous (or native) nucleic acid residue. In some embodiments, the one or more nucleic acid residues that are added can be derived from a heterologous cell or the recombinant host cell itself. In the latter scenario, the nucleic acid residue(s) is (are) added at a genomic location which is different than the native genomic location. The genetic manipulations did not occur in nature and are the results of in vitro manipulations of the native yeast or bacterial host cell.

When expressed in recombinant host cells, the polypeptides (including the enzymes) described herein are encoded on one or more heterologous nucleic acid molecule. The term “heterologous” when used in reference to a nucleic acid molecule (such as a promoter or a coding sequence) refers to a nucleic acid molecule that is not natively found in the recombinant host cell. “Heterologous” also includes a native coding region, or portion thereof, that is removed from the source organism and subsequently reintroduced into the source organism in a form that is different from the corresponding native gene, e.g., not in its natural location in the organism's genome or as additional copies at its natural location. The heterologous nucleic acid molecule is purposively introduced into the recombinant host cell. The term “heterologous” as used herein also refers to an element (nucleic acid or protein) that is derived from a source other than the endogenous source. Thus, for example, a heterologous element could be derived from a different strain of host cell, or from an organism of a different taxonomic group (e.g., different kingdom, phylum, class, order, family genus, or species, or any subgroup within one of these classifications).

When a heterologous nucleic acid molecule is present in the recombinant host cell, it can be integrated in the host cell's genome. The term “integrated” as used herein refers to genetic elements that are placed, through molecular biology techniques, into the genome of a host cell. For example, genetic elements can be placed into the chromosome(s) of the host cell as opposed to in a vector such as a plasmid carried by the host cell. Methods for integrating genetic elements into the genome of a host cell are well known in the art and include homologous recombination. The heterologous nucleic acid molecule can be present in one or more copies in the host cell's chromosome. Alternatively, the heterologous nucleic acid molecule can be independently replicating from the host cell's chromosome. In such embodiment, the nucleic acid molecule can be stable and self-replicating.

In some embodiments, heterologous nucleic acid molecules which can be introduced into the recombinant host cells are codon-optimized with respect to the intended recipient recombinant yeast host cell. As used herein the term “codon-optimized coding region” means a nucleic acid coding region that has been adapted for expression in the cells of a given organism by replacing at least one, or more than one, codons with one or more codons that are more frequently used in the genes of that organism. In general, highly expressed genes in an organism are biased towards codons that are recognized by the most abundant tRNA species in that organism. One measure of this bias is the “codon adaptation index” or “CAI,” which measures the extent to which the codons used to encode each amino acid in a particular gene are those which occur most frequently in a reference set of highly expressed genes from an organism. The CAI of codon optimized heterologous nucleic acid molecule described herein corresponds to between about 0.8 and 1.0, between about 0.8 and 0.9, or about 1.0. In some embodiments, heterologous nucleic acid molecules which can be introduced into the recombinant host cells are codon-optimized with respect to the intended recipient recombinant host cell so as to limit or prevent homologous recombination with the corresponding native gene.

The heterologous nucleic acid molecules of the present disclosure can comprise a coding region for the one or more enzymes to be expressed by the host cell. A DNA or RNA “coding region” is a DNA or RNA molecule which is transcribed and/or translated into a polypeptide in a cell in vitro or in vivo when placed under the control of appropriate regulatory sequences. “Suitable regulatory regions” refer to nucleic acid regions located upstream (5′ non-coding sequences), within, or downstream (3′ non-coding sequences) of a coding region, and which influence the transcription, RNA processing or stability, or translation of the associated coding region. Regulatory regions may include promoters, translation leader sequences, RNA processing sites, effector binding sites and stem-loop structures. The boundaries of the coding region are determined by a start codon at the 5′ (amino) terminus and a translation stop codon at the 3′ (carboxyl) terminus. A coding region can include, but is not limited to, prokaryotic regions, cDNA from mRNA, genomic DNA molecules, synthetic DNA molecules, or RNA molecules. If the coding region is intended for expression in a eukaryotic cell, a polyadenylation signal and transcription termination sequence will usually be located 3′ to the coding region. In an embodiment, the coding region can be referred to as an open reading frame. “Open reading frame” is abbreviated ORF and means a length of nucleic acid, either DNA, cDNA or RNA, that comprises a translation start signal or initiation codon, such as an ATG or AUG, and a termination codon and can be potentially translated into a polypeptide sequence.

The nucleic acid molecules described herein can comprise a non-coding region, for example a transcriptional and/or translational control regions. “Transcriptional and translational control regions” are DNA regulatory regions, such as promoters, enhancers, terminators, and the like, that provide for the expression of a coding region in a host cell. In eukaryotic cells, polyadenylation signals are control regions.

The heterologous nucleic acid molecule can be introduced in the host cell using a vector. A “vector,” e.g., a “plasmid”, “cosmid” or “artificial chromosome” (such as, for example, a yeast artificial chromosome) refers to an extra chromosomal element and is usually in the form of a circular double-stranded DNA molecule. Such vectors may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a host cell.

In the heterologous nucleic acid molecules described herein, the promoter and the nucleic acid molecule coding for the one or more enzymes can be operatively linked to one another. In the context of the present disclosure, the expressions “operatively linked” or “operatively associated” refers to fact that the promoter is physically associated to the nucleotide acid molecule coding for the one or more enzyme in a manner that allows, under certain conditions, for expression of the one or more enzyme from the nucleic acid molecule. In an embodiment, the promoter can be located upstream (5′) of the nucleic acid sequence coding for the one or more enzyme. In still another embodiment, the promoter can be located downstream (3′) of the nucleic acid sequence coding for the one or more enzyme. In the context of the present disclosure, one or more than one promoter can be included in the heterologous nucleic acid molecule. When more than one promoter is included in the heterologous nucleic acid molecule, each of the promoters is operatively linked to the nucleic acid sequence coding for the one or more enzyme. The promoters can be located, in view of the nucleic acid molecule coding for the one or more protein, upstream, downstream as well as both upstream and downstream.

“Promoter” refers to a DNA fragment capable of controlling the expression of a coding sequence or functional RNA. The term “expression,” as used herein, refers to the transcription and stable accumulation of sense (mRNA) from the heterologous nucleic acid molecule described herein.

Expression may also refer to translation of mRNA into a polypeptide. Promoters may be derived in their entirety from a native gene, or be composed of different elements derived from different promoters found in nature, or even comprise synthetic DNA segments. It is understood by those skilled in the art that different promoters may direct the expression at different stages of development, or in response to different environmental or physiological conditions. Promoters which cause a gene to be expressed in most cells at most times at a substantial similar level are commonly referred to as “constitutive promoters”. It is further recognized that since in most cases the exact boundaries of regulatory sequences have not been completely defined, DNA fragments of different lengths may have identical promoter activity. A promoter is generally bounded at its 3′ terminus by the transcription initiation site and extends upstream (5′ direction) to include the minimum number of bases or elements necessary to initiate transcription at levels detectable above background. Within the promoter will be found a transcription initiation site (conveniently defined for example, by mapping with nuclease S1), as well as protein binding domains (consensus sequences) responsible for the binding of the polymerase.

The promoter can be heterologous to the nucleic acid molecule encoding the one or more enzymes. The promoter can be heterologous or derived from a strain being from the same genus or species as the host cell. In an embodiment, the promoter is derived from the same genus or species of the yeast host cell and the heterologous polypeptide is derived from different genus that the host cell.

In some embodiments, the present disclosure concerns the expression of one or more heterologous enzyme, a variant thereof or a fragment thereof in a host cell. A variant comprises at least one amino acid difference when compared to the amino acid sequence of the wild-type heterologous enzyme. The enzyme “variants” have at least 50%, 55%, 60%, 65%, 70%, 75%, 80%, 85%, 90%, 95%, 96%, 97%, 98% or 99% identity to the heterologous enzymes described herein The term “percent identity”, as known in the art, is a relationship between two or more polypeptide sequences or two or more polynucleotide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED=5.

The heterologous enzyme variants exhibit the biological activity associated with the wild-type heterologous enzyme. In an embodiment, the variant enzyme exhibits at least 50%, 55%, 60%, 65%, 70%, 75%, 80%, 85%, 90%, 95%, 96%, 97%, 98% or 99% of the biological activity of the wild-type heterologous enzyme. The biological activity of the heterologous enzymes wild-type and variants can be determined by methods and assays known in the art.

The variant heterologous enzymes described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide.

A “variant” of the enzyme can be a conservative variant or an allelic variant. As used herein, a conservative variant refers to alterations in the amino acid sequence that do not adversely affect the biological functions of the enzyme. A substitution, insertion or deletion is said to adversely affect the protein when the altered sequence prevents or disrupts a biological function associated with the enzyme. For example, the overall charge, structure or hydrophobic-hydrophilic properties of the protein can be altered without adversely affecting a biological activity. Accordingly, the amino acid sequence can be altered, for example to render the peptide more hydrophobic or hydrophilic, without adversely affecting the biological activities of the enzyme.

The heterologous enzyme can be a fragment of a heterologous enzyme or fragment of a variant of a heterologous enzyme. Enzyme “fragments” have at least at least 100, 200, 300, 400, 500 or more consecutive amino acids of the enzyme or the enzyme variant. A fragment comprises at least one less amino acid residue when compared to the amino acid sequence of the wild-type heterologous enzyme. In some embodiments, the fragments corresponding to the native enzyme or enzyme variant to which the signal sequence was removed. In some embodiments, the “fragments” have at least 50%, 55%, 60%, 65%, 70%, 75%, 80%, 85%, 90%, 95%, 96%, 97%, 98% or 99% identity to the enzymes described herein. In some embodiments, fragments of the enzymes can be employed for producing the corresponding full-length enzyme by peptide synthesis. Therefore, the fragments can be employed as intermediates for producing the full-length proteins.

The fragments of heterologous wild-type enzymes or of variants of heterologous enzymes exhibit the biological activity of the heterologous enzyme or the variant. In an embodiment, the fragment enzyme exhibits at least 50%, 55%, 60%, 65%, 70%, 75%, 80%, 85%, 90%, 95%, 96%, 97%, 98% or 99% of the biological activity of the heterologous enzyme or variant thereof. The biological activity of the heterologous enzymes wild-type and variants can be determined by methods and assays known in the art.

In some additional embodiments, the present disclosure also provides reducing the expression of or inactivating a gene ortholog of a gene known to encode a native enzyme. A “gene ortholog” is understood to be a gene in a different species that evolved from a common ancestral gene by speciation. In the context of the present invention, a gene ortholog encodes an enzyme exhibiting the same biological function than the native enzyme.

In some further embodiments, the present disclosure also provides reducing the expression or inactivating a gene paralog of a gene known to encode an enzyme. A “gene paralog” is understood to be a gene related by duplication within the genome. In the context of the present invention, a gene paralog encodes an enzyme that could exhibit additional biological function than the native enzyme.

### Bacterial Host Cell

The combination of the present disclosure comprises a bacterial host cell which is a recombinant bacterial host cell. In an embodiment, the recombinant bacterial host cell can be a Gram-negative bacterial cell. For example, the recombinant bacterial host cell can be from the genus Escherichia (such as for example, from the species Escherichia coli) or from the genus Zymomonas (such as, for example, from the species Zymomonas mobilis). In another embodiment, the recombinant bacterial host cell can be a Gram-positive bacterial cell. In yet another embodiment, the recombinant bacterial host cell can be a lactic acid bacteria or LAB. LAB are a group of Gram-positive bacteria, non-respiring non-spore-forming, cocci or rods, which produce lactic acid as the major end product of the fermentation of carbohydrates. Bacterial genus of LAB include, but are not limited to, Lactobacillus, Leuconostoc, Pediococcus, Lactococcus, Streptococcus, Aerococcus, Carnobacterium, Enterococcus, Oenococcus, Sporolactobacillus, Tetragenococcus, Vagococcus, and Weissella. Bacterial species of LAB include, but are not limited to, Lactococcus lactis, Lactococcus garviae, Lactococcus raffinolactis, Lactococcus pentosus, Oenococcus oeni, Pediococcus pentosaceus, Pediococcus acidilactici, Carnococcus allantoicus, Carnobacterium gallinarum, Vagococcus fessus, Streptococcus thermophilus, Enterococcus phoeniculicola, Enterococcus plantarum, Enterococcus raffinosus, Enterococcus avium, Enterococcus pallens Enterococcus hermanniensis, Enterococcus faecalis, and Enterococcus faecium. In an embodiment, the LAB is a Lactobacillus sp. and, include, without limitation the following genera Lactobacillus delbrueckii group, Paralactobacillus, Holzapfelia, Amylolactobacillus, Bombilactobacillus, Companilactobacillus, Lapidilactobacillus, Agrilactobacillus, Schleiferilactobacillus, Loigolactobacilus, Lacticaseibacillus, Latilactobacillus, Dellaglioa, Liquorilactobacillus, Ligilactobacillus, Lactiplantibacillus, Furfurilactobacillus, Paucilactobacillus, Limosilactobacillus, Fructilactobacillus, Acetilactobacillus, Apilactobacillus, Levilactobacillus, Secundilactobacillus and Lentilactobacillus In some additional embodiments, the Lactobacillus species is L. acetotolerans, L. acidifarinae, L. acidipiscis, L. acidophilus, L. agilis, L. algidus, L. alimentarius, L. amylolyticus, L. amylophilus, L. amylotrophicus, L. amylovorus, L. animalis, L. antri, L. apodemi, L. aviarius, L. bifermentans, L. brevis, L. buchneri, L. camelliae, L. casei, L. catenaformis, L. ceti, L. coleohominis, L. collinoides, L. composti, L. concavus, L. coryniformis, L. crispatus, L. crustorum, L. curvatus, L. delbrueckii (including L. delbrueckii subsp. bulgaricus, L. delbrueckii subsp. delbrueckii, L. delbrueckii subsp. lactis), L. dextrinicus, L. diolivorans, L. equi, L. equigenerosi, L. farraginis, L. farciminis, L. fermentum, L. fornicalis, L. fructivorans, L. frumenti, L. fuchuensis, L. gallinarum, L. gasseri, L. gastricus, L. ghanensis, L. graminis, L. ammesii, L. hamsteri, L. harbinensis, L. hayakitensis, L. helveticus, L. hilgardii, L. omohiochii, L. iners, L. ingluviei, L. intestinalis, L. jensenii, L. johnsonii, L. kalixensis, L. efiranofaciens, L. kefiri, L. kimchii, L. kitasatonis, L. kunkeei, L. leichmannii, L. lindneri, L. alefermentans, L. mali, L. manihotivorans, L. mindensis, L. mucosae, L. murinus, L. nagelii, L. namurensis, L. nantensis, L. oligofermentans, L. oris, L. panis, L. pantheris, L. parabrevis, L. parabuchneri, L. paracasei, L. paracollinoides, L. parafarraginis, L. parakefiri, L. aralimentarius, L. paraplantarum, L. pentosus, L. perolens, L. plantarum, L. pontis, L. protectus, L. psittaci, L. rennini, L. reuteri, L. rhamnosus, L. rimae, L. rogosae, L. rossiae, L. ruminis, L. saerimneri, L. sakei, L. salivarius, L. sanfranciscensis, L. satsumensis, L. secaliphilus, L. sharpeae, L. siliginis, L. spicheri, L. suebicus, L. thailandensis, L. ultunensis, L. vaccinostercus, L. vaginalis, L. versmoldensis, L. vini, L. vitulinus, L. zeae or L. zymae. In some embodiments, the bacterial host cell is from the genus Lactiplantibacillus sp., and in some further embodiments, from the species Lactiplantibacillus pentosus (which was previously referred to as Lactobacillus plantarum or Lactiplantibacillus plantarum).

In the context of the present disclosure, the bacterial host cell has a first metabolic pathway comprising one or more (first) polypeptides of converting acetate into ethanol. In an embodiment, at least one of the first polypeptide of the first metabolic pathway is native. In another embodiment, at least one of the first polypeptide of the first metabolic pathway is heterologous.

In some embodiments, the one or more first polypeptides comprise polypeptides capable of converting (e.g., catalyzing) acetate into acetyl-CoA. The one or first polypeptides can be involved in the conversion of acetate to acetyl phosphate and/or in the conversion of acetyl phosphate in acetyl-CoA. The bacterial host cell can have the intrinsic ability to convert acetate to acetyl phosphate and/or to convert acetyl phosphate in acetyl-CoA. Alternatively or in combination, the bacterial host cell can be engineered to increase the its ability to convert acetate to acetyl phosphate and/or to convert acetyl phosphate in acetyl-CoA (e.g., heterologous). When the bacterial host cell is engineered, the increased in activity in the conversion of acetate to acetyl phosphate and/or the conversion of acetyl phosphate in acetyl-CoA can be caused at least in part by introducing of one or more genetic modifications in a native bacterial host cell to obtain the recombinant bacterial host cell. As such, the activity of the one or more first polypeptides of the recombinant bacterial host cell is considered “increased” because it is higher than the activity of the one or more first polypeptides in the native bacterial host cell (e.g., prior to the introduction of the one or more genetic modifications). The one or more genetic modifications is not limited to a specific modification provided that it does increase the activity, and in some embodiments, the expression of the one or more first polypeptide and ultimately the conversion of acetate into acetyl-CoA. For example, the one or more genetic modifications can include the addition of a promoter to increase the expression of the one or more (native) first polypeptide. Alternatively or in addition, the one or more genetic modifications can include the introduction of one or more copies of a gene encoding the one or more first (heterologous) polypeptide in the recombinant bacterial host cell.

The one or more first polypeptides comprise, in some embodiments, a polypeptide having acetate kinase (ACK) activity, a polypeptide having a phosphotransacetylase (PTA) activity, and/or a polypeptide having acetyl-CoA synthetase (ACS) activity. In an embodiment, the bacterial host cell of the present disclosure comprises a polypeptide having acetate kinase (ACK) activity. The polypeptide having acetate kinase (ACK) activity can be native to the bacterial host cell or can be genetically engineered in the bacterial host cell (heterologous). In another embodiment, the bacterial host cell of the present disclosure comprises a polypeptide having phosphotransacetylase (PTA) activity. The polypeptide having phosphotransacetylase (PTA) activity can be native to the bacterial host cell or can be genetically engineered in the bacterial host cell (heterologous). In an embodiment, the bacterial host cell of the present disclosure comprises a polypeptide having acetyl-CoA synthetase (ACS) activity. The polypeptide having acetyl-CoA synthetase (ACS) activity can be native to the bacterial host cell or can be genetically engineered in the bacterial host cell (heterologous). In still another embodiment, the bacterial host cell of the present disclosure comprises a polypeptide having acetate kinase (ACK) activity and a polypeptide having a phosphotransacetylase (PTA) activity. In some embodiments, the polypeptide having acetate kinase (ACK) activity and the polypeptide having a phosphotransacetylase (PTA) activity are both native to the bacterial host cell. In additional embodiments, the polypeptide having acetate kinase (ACK) activity and the polypeptide having a phosphotransacetylase (PTA) activity are both heterologous to the bacterial host cell. In further embodiments, at least one of the polypeptide having acetate kinase (ACK) activity and the polypeptide having a phosphotransacetylase (PTA) activity is native to the bacterial host cell. In still another embodiment, the bacterial host cell of the present disclosure comprises a polypeptide having acetate kinase (ACK) activity, a polypeptide having a phosphotransacetylase (PTA) activity, and a polypeptide having acetyl-CoA synthetase (ACS) activity. In some embodiments, the polypeptide having acetate kinase (ACK) activity, the polypeptide having a phosphotransacetylase (PTA) activity, and the polypeptide having acetyl-CoA synthetase (ACS) activity are all native to the bacterial host cell. In additional embodiments, the polypeptide having acetate kinase (ACK) activity, the polypeptide having a phosphotransacetylase (PTA) activity, and the polypeptide having acetyl-CoA synthetase (ACS) activity are all heterologous to the bacterial host cell. In further embodiments, at least one of the polypeptide having acetate kinase (ACK) activity, the polypeptide having a phosphotransacetylase (PTA) activity, or the polypeptide having acetyl-CoA synthetase (ACS) activity is native to the bacterial host cell.

Polypeptides having a polypeptide having acetate kinase (ACK) activity include, but are not limited to an acetate kinase (ACK). Acetate kinases are involved in the conversion of acetate and ATP into acetyl phosphate and ADP. In the bacterial host cell of the present disclosure, the acetate kinase can be of prokaryotic or eukaryotic origin. In some embodiments, the acetate kinase can be native or heterologous to the bacterial host cell. In an embodiment, the acetate kinase can be obtained from or derived from Lactiplantibacillus sp. and in some embodiments from Lactiplantibacillus pentosus. The acetate kinase can have, in some embodiments, the amino acid sequence of SEQ ID NO: 25 or 27, be a variant of the amino acid sequence of SEQ ID NO: 25 or 27 having acetate kinase activity or be a fragment of the amino acid sequence of SEQ ID NO: 25 or 27. In some additional embodiments, the acetate kinase can be encoded by a nucleic acid sequence having the nucleic acid sequence of SEQ ID NO: 26 or 28 or comprising a degenerate sequence encoding the amino acid sequence of SEQ ID NO: 25 or 27.

Polypeptides having phosphotransacetylase (PTA) activity include, but are not limited to, a phosphotransacetylase. Phosphotransacetylases are involved in the conversion of acetyl phosphate and CoA into acetyl-CoA and Pi. In the bacterial host cell of the present disclosure, the phosphotransacetylase can be of prokaryotic or eukaryotic origin. In some embodiments, the phosphotransacetylase can be native or heterologous to the bacterial host cell. In an embodiment, the phosphotransacetylase can be obtained from or derived from Lactiplantibacillus sp. and in some embodiments from Lactiplantibacillus pentosus. In some embodiments, the phosphotransacetylase can have the amino acid sequence of SEQ ID NO: 29, be a variant of the amino acid sequence of SEQ ID NO: 29 having phosphotranscetylase activity or be a fragment of the amino acid sequence of SEQ ID NO: 29 having phosphotransacetylase activity. In another embodiment, the phosphotranscetylase can be encoded by a nucleic acid molecule comprising the nucleic acid sequence of SEQ ID NO: 30 or comprising a degenerate sequence encoding the amino acid sequence of SEQ ID NO: 29.

Polypeptides having acetyl-CoA synthetase (ACS) activity include, but are not limited to, an acetyl-CoA synthetase (which is also known as an acetate-CoA ligase). Acetyl-CoA synthetase are involved in the conversion of acetate and ATP into AMP, pyrophosphate and acetyl-CoA. In the bacterial host cell of the present disclosure, the acetyl-CoA synthetase can be of prokaryotic or eukaryotic origin. In some embodiments, the acetyl-CoA synthetase can be native or heterologous to the bacterial host cell. In an embodiment, the acetyl-CoA synthetase can be obtained from or derived from Lactiplantibacillus sp. and in some embodiments from Lactiplantibacillus pentosus. In some further embodiments, the acetyl-CoA synthetase can be obtained or derived from Salmonella sp., such as, for example, from Salmonella enterica. In some embodiments, the acetyl-CoA synthetase can have the amino acid sequence of SEQ ID NO: 75, be a variant of the amino acid sequence of SEQ ID NO: 75 having phosphotranscetylase activity or be a fragment of the amino acid sequence of SEQ ID NO: 75 having phosphotransacetylase activity. In another embodiment, the phosphotranscetylase can be encoded by a nucleic acid molecule comprising a degenerate sequence encoding the amino acid sequence of SEQ ID NO: 75. In some further embodiments, the acetyl-CoA synthetase is obtained from or derived from Zygosaccharomyces sp., such as, for example, from Zygosaccharomyces bailii. In some embodiments, the acetyl-CoA synthetase can have the amino acid sequence of SEQ ID NO: 76, be a variant of the amino acid sequence of SEQ ID NO: 76 having phosphotranscetylase activity or be a fragment of the amino acid sequence of SEQ ID NO: 76 having phosphotransacetylase activity. In another embodiment, the phosphotranscetylase can be encoded by a nucleic acid comprising a degenerate sequence encoding the amino acid sequence of SEQ ID NO: 76. In some further embodiments, the acetyl-CoA synthetase is obtained from or derived from Acetobacter sp., such as, for example, from Acetobacter aceti. In some embodiments, the acetyl-CoA synthetase can have the amino acid sequence of SEQ ID NO: 77, be a variant of the amino acid sequence of SEQ ID NO: 77 having phosphotranscetylase activity or be a fragment of the amino acid sequence of SEQ ID NO: 77 having phosphotransacetylase activity. In another embodiment, the phosphotranscetylase can be encoded by a nucleic acid molecule comprising a degenerate sequence encoding the amino acid sequence of SEQ ID NO: 77. In some further embodiments, the acetyl-CoA synthetase is obtained from or derived from Saccharomyces sp., such as, for example, from Saccharomyces cerevisiae. In some embodiments, the acetyl-CoA synthetase can have the amino acid sequence of SEQ ID NO: 78 or 79, be a variant of the amino acid sequence of SEQ ID NO: 78 or 79 having phosphotranscetylase activity or be a fragment of the amino acid sequence of SEQ ID NO: 78 or 79 having phosphotransacetylase activity. In another embodiment, the phosphotranscetylase can be encoded by a nucleic acid molecule comprising a degenerate sequence encoding the amino acid sequence of SEQ ID NO: 78 or 79.

In an embodiment, the one or more first polypeptides include a polypeptide capable of converting (e.g., catalyzing) acetyl-CoA into acetaldehyde (and optionally acetaldehyde into ethanol). The one or more first polypeptides can be involved in the conversion of acetyl phosphate into acetaldehyde or in the conversion of acetaldehyde into ethanol or both. In some embodiments, the one or more polypeptides capable of converting acetyl-CoA into acetaldehyde (and optionally acetaldehyde into ethanol) can be native or heterologous to the bacterial host cell. The bacterial host cell of the present disclosure can be engineered to increase the activity in the one or more first polypeptide capable of converting acetyl-CoA into acetaldehyde (and optionally acetaldehyde into ethanol). The increased in activity in the capacity in converting acetyl-CoA into acetaldehyde (and optionally acetaldehyde into ethanol) can be caused, at least in part, by introducing of one or more genetic modifications in a native bacterial host cell to obtain the recombinant bacterial host cell. As such, the activity of converting acetyl-CoA into acetaldehyde (and optionally acetaldehyde into ethanol) of the recombinant bacterial host cell is considered “increased” because it is higher than the corresponding activity in the native bacterial host cell (e.g., prior to the introduction of the one or more genetic modifications). The one or more genetic modifications are not limited to a specific modification provided that it does increase the activity, and in some embodiments, the expression of the one or more polypeptide capable of converting acetyl-CoA into acetaldehyde (and optionally acetaldehyde into ethanol). For example, the one or more genetic modifications can include the addition of a promoter to increase the expression of the one or more polypeptides capable of converting acetyl-CoA into acetaldehyde (and optionally acetaldehyde into ethanol). Alternatively or in addition, the one or more genetic modifications can include the introduction of one or more copies of a gene encoding the one or more polypeptide capable of converting acetyl-CoA and/or acetaldehyde into ethanol in the recombinant bacterial host cell.

The one or more first polypeptide capable of converting acetyl-CoA into acetaldehyde (and optionally acetaldehyde into ethanol) can include, without limitation, a polypeptide having an acetylating acetaldehyde dehydrogenase (AADH) activity, a polypeptide having an alcohol dehydrogenase activity and/or a polypeptide having a bifunctional acetylating acetaldehyde/alcohol dehydrogenase (ADHE) activity. In an embodiment, the bacterial host cell of the present disclosure comprises a polypeptide having acetylating acetaldehyde dehydrogenase activity. In another embodiment, the bacterial host cell of the present disclosure comprises a polypeptide having an alcohol dehydrogenase activity. In a further embodiment, the bacterial host cell of the present disclosure comprises a polypeptide having an alcohol dehydrogenase activity and a polypeptide having an alcohol dehydrogenase activity. In yet another embodiment, the bacterial host cell of the present disclosure comprises a bifunctional acetaldehyde/alcohol dehydrogenase (ADHE) activity.

Polypeptides having acetylating acetaldehyde dehydrogenase (AADH) activity include, but are not limited to, an acetaldehyde dehydrogenase (EC 1.1.1.1). Acetaldehyde dehydrogenases are involved in the conversion of acetyl-CoA and NADH into acetaldehyde, NAD⁺ and CoA. In the bacterial host cell, the acetaldehyde dehydrogenase can be of prokaryotic or eukaryotic origin. In some embodiments, the acetaldehyde dehydrogenase can be native or heterologous to the bacterial host cell. In an embodiment, the acetaldehyde dehydrogenase can be obtained from or derived from Lactiplantibacillus sp. and in some embodiments from Lactiplantibacillus pentosus. Polypeptides having alcohol dehydrogenase (ADH) activity include, but are not limited to an alcohol dehydrogenase (EC 1.1.1.1). Alcohol dehydrogenases are involved in the conversion of acetaldehyde and NADH into ethanol and NAD⁺. In the bacterial host cell, the alcohol dehydrogenase can be of prokaryotic or eukaryotic origin. In some embodiments, the alcohol dehydrogenase can be native or heterologous to the bacterial host cell. Alcohol dehydrogenases include, but are not limited to, ADH4 from Saccharomyces cerevisiae, ADHB from Zymonas mobilis, FUCO from Escherichia coli, ADHE from Escherichia coli, ADH1 from Clostridium acetobutylicum, ADH1 from Entamoeba nuttalli, BDHA from Clostridium acetobutylicum, BDHB from Clostridium acetobutylicum, 4H BID from Clostridium kluyveri, DHAT from Citrobacter freundii or DHAT from Klebsiella pneumoniae. In an embodiment, the alcohol dehydrogenase can be ADHB from Zymonas mobilis (Gene ID: AHJ71151.1), Lactobacillus reuteri (Accession Number: KRK51011.1), Lactobacillus mucosae (Accession Number WP_048345394.1), Lactobacillus brevis (Accession Number WP_003553163.1) or Streptococcus thermophiles (Accession Number WP_113870363.1). In an embodiment, the alcohol dehydrogenase can be obtained from or derived from Zymomomas sp. and in some embodiments from Zymomonas mobilis. In an embodiment, the alcohol dehydrogenase can be obtained from or derived from Lactiplantibacillus sp. and in some embodiments from Lactiplantibacillus pentosus. In an embodiment, the alcohol dehydrogenase comprises the amino acid sequence of SEQ ID NO: 18, is a variant of the amino acid sequence of SEQ ID NO: 18 having alcohol dehydrogenase activity or is a fragment of the amino acid sequence of SEQ ID NO: 18 having alcohol dehydrogenase activity. In yet another embodiment, the alcohol dehydrogenase can be encoded by a nucleic acid molecule having the nucleic acid sequence of SEQ ID NO: 19 or 20 be a degenerate sequence encoding the amino acid sequence of SEQ ID NO: 18.

Polypeptides having both acetylating acetaldehyde dehydrogenase (AADH) activity as well as alcohol dehydrogenase activity include, but are not limited to, a bifunctional acetylating acetaldehyde/alcohol dehydrogenase (EC 1.1.1.1). Acetylating dehydrogenases are involved in the conversion of acetyl-CoA and NADH into acetaldehyde, NAD⁺ and CoA. In the bacterial host cell, the acetaldehyde/alcohol dehydrogenase can be of prokaryotic or eukaryotic origin. In some embodiments, the acetaldehyde/alcohol dehydrogenase can be native or heterologous to the bacterial host cell. Bifunctional acetaldehyde/alcohol dehydrogenases such as those described in U.S. Pat. No. 8,956,851 and US Patent Application published under US2016/0194669, both of which are incorporated herewith in their entirety. In an embodiment, the bifunctional acetylating acetaldehyde/alcohol dehydrogenase is from Lactiplantibacillus sp. and in some further embodiments, from Lactiplantibacillus pentosus. In additional embodiments, the bifunctional acetylating acetaldehyde/alcohol dehydrogenase comprises the amino acid sequence of SEQ ID NO: 31, 33 or 55, is a variant of the amino acid sequence of SEQ ID NO: 31, 33 or 55 having bifunctional acetylating acetaldehyde/alcohol dehydrogenase activity or is a fragment of the amino acid sequence of SEQ ID NO: 31, 33 or 55 having bifunctional acetylating acetaldehyde/alcohol dehydrogenase activity. In some further embodiments, the bifunctional acetylating acetaldehyde/alcohol dehydrogenase is encoded by a nucleic acid molecule having the nucleic acid sequence of SEQ ID NO: 32 or 34 or comprises a degenerate sequence encoding the amino acid sequence of SEQ ID NO: 31 or 33.

The bacterial host cell of the present disclosure comprises one or more second polypeptides for the conversion of glycerol into dihydroxyacetone phosphate (which can, in some embodiments, be for the dehydrogenation of glycerol). The bacterial host cell can have the intrinsic activity in the conversion of glycerol into dihydroxyacetone phosphate (e.g., a native second metabolic pathway). Alternatively, the bacterial host cell can be engineered to increase the activity in one or more second polypeptides in the second metabolic pathway (e.g., a heterologous second metabolic pathway). The activity in the glycerol dehydrogenation pathway can, in some embodiments, be increased or observed only when the bacterial host cell is placed in anaerobic conditions. When the second metabolic pathway is engineered, the increased in activity in the second metabolic pathway can be caused, at least in part, by introducing of one or more genetic modifications in a native bacterial host cell to obtain the recombinant bacterial host cell. As such, the activity of the one or more second polypeptides of the recombinant bacterial host cell is considered “increased” because it is higher than the activity of the one or more second polypeptides in the native bacterial host cell (e.g., prior to the introduction of the one or more second genetic modifications). The one or more second genetic modifications are not limited to a specific modification provided that it does increase the activity, and in some embodiments, the expression of the one or more second polypeptides and ultimately activity in the metabolic pathway for the conversion of glycerol into dihydroxyacetone phosphate. For example, the one or more genetic modifications can include the addition of a promoter to increase the expression of the one or more (native) second polypeptides. Alternatively or in addition, the one or more genetic modifications can include the introduction of one or more copies of a gene(s) encoding the one or more second (heterologous) polypeptides in the recombinant bacterial host cell.

In some embodiments, the one or more second polypeptides comprise a polypeptide having glycerol dehydrogenase (GLDA) activity, a polypeptide having an ATP-dependent dihydroxyacetone kinase (DAK) activity and/or a polypeptide having a PEP-dependent dihydroxyacetone kinase (DHALM) activity. In one embodiment, the bacterial host cell of the present disclosure comprises a polypeptide having glycerol dehydrogenase (GLDA) activity. In another embodiment, the bacterial host cell of the present disclosure comprises a polypeptide having an ATP-dependent dihydroxyacetone kinase (DAK) activity. In another embodiment, the bacterial host cell of the present disclosure comprises a polypeptide having a PEP-dependent dihydroxyacetone kinase (DHAKLM) activity. In still another embodiment, the bacterial host cell of the present disclosure comprises a polypeptide having glycerol dehydrogenase (GLDA) activity and a polypeptide having an ATP-dependent dihydroxyacetone kinase (DAK) activity. In yet another embodiment, the bacterial host cell of the present disclosure comprises a polypeptide having glycerol dehydrogenase (GLDA) activity and a polypeptide having a PEP-dependent dihydroxyacetone kinase (DHAKLM) activity. In still another embodiment, the bacterial host cell of the present disclosure comprises a polypeptide having an ATP-dependent dihydroxyacetone kinase (DAK) activity and a polypeptide having a PEP-dependent dihydroxyacetone kinase (DHAKLM) activity. In yet a further embodiment, the bacterial host cell of the present disclosure comprises a polypeptide having glycerol dehydrogenase (GLDA) activity, a polypeptide having an ATP-dependent dihydroxyacetone kinase (DAK) activity and a polypeptide having a PEP-dependent dihydroxyacetone kinase (DHAKLM) activity. The one or more second polypeptide can include a polypeptide having glycerol dehydrogenase activity, such as a glycerol dehydrogenase (E.C. 1.1.1.6). Glycerol dehydrogenase activity can be determined by any assays or methods in the art including those described in Tang et al., 1979. Glycerol dehydrogenases are involved in the conversion of glycerol and NAD⁺ into dihydroxyacetone and NADH. In the bacterial host cell, the glycerol dehydrogenase can be of prokaryotic or eukaryotic origin. In some embodiments, the glycerol dehydrogenase can be native or heterologous to the bacterial host cell. In specific embodiments, the bacterial host cell can comprise a native glycerol dehydrogenase and a heterologous glycerol dehydrogenase. In some embodiments, the glycerol dehydrogenase can be native or heterologous to the bacterial host cell. In an embodiment, the glycerol dehydrogenase can be obtained from or derived from Lactiplantibacillus sp. and in some embodiments from Lactiplantibacillus pentosus. In embodiments in which the recombinant bacterial host cell is Lactiplanticallus pentosus or Lacticaseibacillus paracasei, the glycerol dehydrogenase can be obtained from or derived from Lactiplantibacillus sp. and in some embodiments from Lactiplantibacillus pentosus. The glycerol dehydrogenase can have, in some embodiments, the amino acid sequence of SEQ ID NO: 7, be a variant of the amino acid sequence of SEQ ID NO: 7 having glycerol dehydrogenase activity or be a fragment of the amino acid sequence of SEQ ID NO: 7 having glycerol dehydrogenase activity. The glycerol dehydrogenase can be encoded by a nucleic acid sequence having the nucleic acid sequence of SEQ ID NO: 8, 80 or 87 or a degenerate sequence encoding the amino acid sequence of SEQ ID NO: 7. In an embodiment, the glycerol dehydrogenase can be obtained from or derived from Escherichia sp. and in some embodiments from Escherichia coli. The glycerol dehydrogenase can have, in some embodiments, the amino acid sequence of SEQ ID NO: 44, be a variant of the amino acid sequence of SEQ ID NO: 44 having glycerol dehydrogenase activity or be a fragment of the amino acid sequence of SEQ ID NO: 44 having glycerol dehydrogenase activity. The glycerol dehydrogenase can be encoded by a nucleic acid sequence comprising a degenerate sequence encoding the amino acid sequence of SEQ ID NO: 44. In an embodiment, the glycerol dehydrogenase can be obtained from or derived from Enterococcus sp. The glycerol dehydrogenase can have, in some embodiments, the amino acid sequence of SEQ ID NO: 21, be a variant of the amino acid sequence of SEQ ID NO: 21 having glycerol dehydrogenase activity or be a fragment of the amino acid sequence of SEQ ID NO: 21 having glycerol dehydrogenase activity. The glycerol dehydrogenase can be encoded by a nucleic acid sequence comprising a degenerate sequence encoding the amino acid sequence of SEQ ID NO: 21.

The one or more second polypeptides in the second metabolic pathway can include a polypeptide having ATP-dependent dihydroxyacetone kinase (DAK) activity, such as an ATP-dependent dihydroxyacetone kinase (DAK). ATP-dependent dihydroxyacetone kinases are involved in the conversion of dihydroxyacetone and ATP into dihydroxyacetone phosphate and ADP. In the bacterial host cell, the ATP-dependent dihydroxyacetone kinase can be of prokaryotic or eukaryotic origin. In some embodiments, the ATP-dependent dihydroxyacetone kinase can be native or heterologous to the bacterial host cell. In an embodiment, the ATP-dependent dihydroxyacetone kinase (DAK) can be obtained from or derived from Saccharomyces sp. and in some embodiments from Saccharomyces cerevisiae. In an embodiment, the ATP-dependent dihydroxyacetone kinase (DAK) can be obtained from or derived from Lactiplantibacillus sp. and in some embodiments from Lactiplantibacillus pentosus. The ATP-dependent dihydroxyacetone kinase can have, in some embodiments, the amino acid sequence of SEQ ID NO: 43, be a variant of the amino acid sequence of SEQ ID NO: 43 having ATP-dependent dihydroxyacetone kinase activity or be a fragment of the amino acid sequence of SEQ ID NO: 43 having ATP-dependent dihydroxyacetone kinase activity. The ATP-dependent dihydroxyacetone kinase can be encoded by a nucleic acid sequence comprising a degenerate sequence encoding the amino acid sequence of SEQ ID NO: 43.

The one or more second polypeptides can include a polypeptide having PEP-dependent dihydroxyacetone kinase activity, such as a PEP-dependent dihydroxyacetone kinase. PEP-dependent dihydroxyacetone kinases are involved in the conversion of dihydroxyacetone and PEP into dihydroxyacetone phosphate and pyruvate. In some embodiments, the PEP-dependent dihydroxyacetone kinases are multimeric (and can include, for example, a first kinase (which can be referred to as DHAK), a second ADP-binding subunity (which can be referred to as DHAL) and a third phosphoenolpyruvate-dihydroxyacetone phosphotransferase subunit (which can be referred to as DHAM)). In the bacterial host cell, the PEP-dependent dihydroxyacetone kinase can be of prokaryotic or eukaryotic origin. In some embodiments, the PEP-dependent dihydroxyacetone kinase can be native or heterologous to the bacterial host cell. In an embodiment, the PEP-dependent dihydroxyacetone kinase can be obtained from or derived from Lactiplantibacillus sp. and in some embodiments from Lactiplantibacillus pentosus. In embodiments in which the recombinant bacterial host cell is Lactiplanticallus pentosus, the PEP-dependent dihydroxyacetone kinase can be obtained from or derived from Lactiplantibacillus sp. and in some embodiments from Lactiplantibacillus pentosus. The PEP-dependent dihydroxyacetone kinase can have, in some embodiments, the amino acid sequence of SEQ ID NO: 9, 11 or 13, be a variant of the amino acid sequence of SEQ ID NO: 9, 11 or 13 having a PEP-dependent dihydroxyacetone kinase activity or be a fragment of the amino acid sequence of SEQ ID NO: 9, 11 or 13 having a PEP-dependent dihydroxyacetone kinase activity. The PEP-dependent dihydroxyacetone kinase can be encoded by a nucleic acid sequence having the nucleic acid sequence of SEQ ID NO: 10, 12, 14, 81, 82, or 83 or a degenerate sequence encoding the amino acid sequence of SEQ ID NO: 9, 11 or 13. In an embodiment, the PEP-dependent dihydroxyacetone kinase can be obtained from or derived from Lacticaseibacillus sp. and in some embodiments from Lacticaseibacillus paracasei. In some embodiments, when the recombinant bacterial host cell is a Lacticaseibacillus paracasei, the PEP-dependent dihydroxyacetone kinase can be obtained from or derived from Lacticaseibacillus sp. and in some embodiments from Lacticaseibacillus paracasei. The PEP-dependent dihydroxyacetone kinase can have, in some embodiments, the amino acid sequence of SEQ ID NO: 84, 85, or 86, be a variant of the amino acid sequence of SEQ ID NO: 84, 85, 86 having a PEP-dependent dihydroxyacetone kinase activity or be a fragment of the amino acid sequence of SEQ ID NO: 84, 85, or 86 having a PEP-dependent dihydroxyacetone kinase activity. The PEP-dependent dihydroxyacetone kinase can be encoded by a nucleic acid sequence having the nucleic acid sequence of SEQ ID NO: 88, 89, or 90 or a degenerate sequence encoding the amino acid sequence of SEQ ID NO: 84, 85, or 86. In an embodiment, the PEP-dependent dihydroxyacetone kinase can be obtained from or derived from Enterococcus sp. The PEP-dependent dihydroxyacetone kinase can have, in some embodiments, the amino acid sequence of SEQ ID NO: 22, 23 or 24, be a variant of the amino acid sequence of SEQ ID NO: 22, 23 or 24 having a PEP-dependent dihydroxyacetone kinase activity or be a fragment of the amino acid sequence of SEQ ID NO: 22, 23 or 24 having a PEP-dependent dihydroxyacetone kinase activity. The PEP-dependent dihydroxyacetone kinase can be encoded by a nucleic acid sequence comprising degenerate sequence encoding the amino acid sequence of SEQ ID NO: 22, 23 or 24.

In some specific embodiments, the recombinant bacterial host cell comprises both a heterologous glycerol dehydrogenase and a heterologous PEP-dependent dihydroxyacetone kinase. In such embodiment, the recombinant bacterial host cell can already have a native glycerol dehydrogenase and/or a native PEP-dependent dihydroxyacetone kinase. The recombinant bacterial host cell comprising both a heterologous glycerol dehydrogenase and a heterologous PEP-dependent dihydroxyacetone kinase can have a distinct operons for expressing the heterologous glycerol dehydrogenase and the heterologous PEP-dependent dihydroxyacetone kinase. Alternatively, the recombinant bacterial host cell comprising both a heterologous glycerol dehydrogenase and a heterologous PEP-dependent dihydroxyacetone kinase can have a single operon for expressing the heterologous glycerol dehydrogenase and the heterologous PEP-dependent dihydroxyacetone kinase. In some embodiments, when the recombinant bacterial host cell is Lactiplantibacillus pentosus, it can comprise an heterologous glycerol dehydrogenase having, in some embodiments, the amino acid sequence of SEQ ID NO: 7, being a variant of the amino acid sequence of SEQ ID NO: 7 having glycerol dehydrogenase activity or being a fragment of the amino acid sequence of SEQ ID NO: 7 having glycerol dehydrogenase activity. The glycerol dehydrogenase can be encoded by a heterologous nucleic acid sequence having the nucleic acid sequence of SEQ ID NO: 8, or 80 or a degenerate sequence encoding the amino acid sequence of SEQ ID NO: 7. In some embodiments, when the recombinant bacterial host cell is Lactiplantibacillus pentosus, it can comprise an heterologous PEP-dependent dihydroxyacetone kinase having, in some embodiments, the amino acid sequence of SEQ ID NO: 9, 11 or 13, being a variant of the amino acid sequence of SEQ ID NO: 9, 11 or 13 having a PEP-dependent dihydroxyacetone kinase activity or being a fragment of the amino acid sequence of SEQ ID NO: 9, 11 or 13 having a PEP-dependent dihydroxyacetone kinase activity. The PEP-dependent dihydroxyacetone kinase can be encoded by a heterologous nucleic acid sequence having the nucleic acid sequence of SEQ ID NO: 10, 12, or 14 or a degenerate sequence encoding the amino acid sequence of SEQ ID NO: 9, 11 or 13. In some embodiments, when the recombinant bacterial host cell is Lacticaseibacillus paracasei, it can comprise an heterologous glycerol dehydrogenase having, in some embodiments, the amino acid sequence of SEQ ID NO: 7, being a variant of the amino acid sequence of SEQ ID NO: 7 having glycerol dehydrogenase activity or being a fragment of the amino acid sequence of SEQ ID NO: 7 having glycerol dehydrogenase activity. The glycerol dehydrogenase can be encoded by a heterologous nucleic acid sequence having the nucleic acid sequence of SEQ ID NO: 87 or a degenerate sequence encoding the amino acid sequence of SEQ ID NO: 7. In some embodiments, when the recombinant bacterial host cell is Lacticaseibacillus paracasei, it can comprise an heterologous PEP-dependent dihydroxyacetone kinase having, in some embodiments, the amino acid sequence of SEQ ID NO: 84, 85, or 86, being a variant of the amino acid sequence of SEQ ID NO: 84, 85, or 86 having a PEP-dependent dihydroxyacetone kinase activity or being a fragment of the amino acid sequence of SEQ ID NO: 84, 85, or 86 having a PEP-dependent dihydroxyacetone kinase activity. The PEP-dependent dihydroxyacetone kinase can be encoded by a heterologous nucleic acid sequence having the nucleic acid sequence of SEQ ID NO: 88, 89, or 90 or a degenerate sequence encoding the amino acid sequence of SEQ ID NO: 88, 89, or 90.

In additional embodiments, the bacterial host cell of the present disclosure can include one or more native or heterologous polypeptide capable of transporting and/or facilitating glycerol inside the bacterial cell (e.g., glycerol uptake). Polypeptides capable of transporting glycerol inside the bacterial cell can include, without limitations, GLDF polypeptides as well as variants and fragments thereof exhibiting glycerol transport activity. In embodiments, the GLDF polypeptides are derived from Lactiplantibacillus sp., such as, for example, from Lactiplantibacillus pentosus. In an embodiment, the GLDF polypeptide comprises the amino acid sequence of SEQ ID NO: 35, is a variant of the amino acid sequence of SEQ ID NO: 35 and having the ability to facilitate glycerol transport or is a fragment of the amino acid sequence of SEQ ID NO: 35 and having the ability to facilitate glycerol transport. In some embodiments, the GLDF polypeptide is encoded by a nucleic acid molecule having the nucleic acid sequence of SEQ ID NO: 36 or comprises a degenerate sequence encoding the amino acid sequence of SEQ ID NO: 35. In an embodiment, the GLDF polypeptide comprises the amino acid sequence of SEQ ID NO: 37, is a variant of the amino acid sequence of SEQ ID NO: 37 and having the ability to facilitate glycerol transport or is a fragment of the amino acid sequence of SEQ ID NO: 37 and having the ability to facilitate glycerol transport. In some embodiment, the GLDF polypeptide is encoded by a nucleic acid molecule having the nucleic acid sequence of SEQ ID NO: 38 or comprises a degenerate sequence encoding the amino acid sequence of SEQ ID NO: 37. In an embodiment, the GLDF polypeptide comprises the amino acid sequence of SEQ ID NO: 39, is a variant of the amino acid sequence of SEQ ID NO: 39 and having the ability to facilitate glycerol transport or is a fragment of the amino acid sequence of SEQ ID NO: 39 and having the ability to facilitate glycerol transport. In some embodiment, the GLDF polypeptide is encoded by a nucleic acid molecule having the nucleic acid sequence of SEQ ID NO: 40 or comprises a degenerate sequence encoding the amino acid sequence of SEQ ID NO: 39. In an embodiment, the GLDF polypeptide comprises the amino acid sequence of SEQ ID NO: 41, is a variant of the amino acid sequence of SEQ ID NO: 41 and having the ability to facilitate glycerol transport or is a fragment of the amino acid sequence of SEQ ID NO: 41 and having the ability to facilitate glycerol transport. In some embodiment, the GLDF polypeptide is encoded by a nucleic acid molecule having the nucleic acid sequence of SEQ ID NO: 42 or comprises a degenerate sequence encoding the amino acid sequence of SEQ ID NO: 41.

The accumulation of dihydroxyacetone phosphate will generate, during glycolysis, pyruvate which can be converted to ethanol. The bacterial host cell of the present disclosure thus has a third metabolic pathway comprising one or more third polypeptides of converting pyruvate into ethanol. The third metabolic pathway can be native or heterologous in the bacterial host cell. The bacterial host cell of the present disclosure can be engineered to increase the activity in one or more third polypeptide in the third metabolic pathway (e.g., a heterologous third metabolic pathway). The increased in activity in the third metabolic pathway can be caused, at least in part, by introducing of one or more genetic modifications in a native bacterial host cell to obtain the recombinant bacterial host cell. As such, the activity of the one or more third heterologous polypeptide of the recombinant bacterial host cell is considered “increased” because it is higher than the activity of the one or more third polypeptides in the native bacterial host cell (e.g., prior to the introduction of the one or more genetic modifications). The one or more genetic modifications are not limited to a specific modification provided that it does increase the activity, and in some embodiments, the expression of the one or more third polypeptides and ultimately converting pyruvate into ethanol. For example, the one or more genetic modifications can include the introduction of one or more copies of a gene(s) encoding the one or more third heterologous polypeptides in the recombinant bacterial host cell.

The one or more polypeptides in the third metabolic pathway can include a polypeptide having pyruvate decarboxylase activity, such as, for example a pyruvate decarboxylase (EC 4.1.1.1). Pyruvate decarboxylases are involved in the conversion of pyruvate into acetaldehyde and CO₂. In the bacterial host cell, the pyruvate decarboxylase (PDC) can be of prokaryotic or eukaryotic origin. Pyruvate decarboxylases can be derived, for example, from Lactobacillus florum (Accession Number WP_009166425.1), Lactobacillus fructivorans (Accession Number WP_039145143.1), Lactobacillus lindneri (Accession Number WP_065866149.1), Lactococcus lactis (Accession Number WP_104141789.1), Carnobacterium gallinarum (Accession Number WP_034563038.1), Enterococcus plantarum (Accession Number WP_069654378.1), Clostridium acetobutylicum (Accession Number NP_149189.1), Bacillus megaterium (Accession Number WP_075420723.1), and/or Bacillus thuringiensis (Accession Number WP_052587756.1). In an embodiment, the pyruvate decarboxylase can be from Zymomonas sp. and in some further embodiments, from Zymomomas mobilis. In an embodiment, the pyruvate decarboxylase can be from Lactiplantibacillus sp., such as, for example, from Lactiplantibacillus pentosus. In an embodiment, the pyruvate decarboxylase comprises the amino acid sequence of SEQ ID NO: 15, is a variant of the amino acid sequence of SEQ ID NO: 15 having pyruvate decarboxylase activity or is a fragment of the amino acid sequence of SEQ ID NO: 15 having pyruvate decarboxylase activity. In yet another embodiment, the pyruvate decarboxylase can be encoded by a nucleic acid molecule having the nucleic acid sequence of SEQ ID NO: 16 or 17 be a degenerate sequence encoding the amino acid sequence of SEQ ID NO: 15. In an embodiment, more than one heterologous nucleic acid molecules encoding a pyruvate decarboxylase are incorporated in the recombinant bacterial host cell. In some embodiments, at least two heterologous nucleic acid molecules encoding a pyruvate decarboxylase are incorporated in the recombinant bacterial host cell. For example, the at least two heterologous nucleic acid molecules encoding a pyruvate decarboxylase can be incorporated at two different loci and each of the expression of the pyruvate decarboxylase gene is under the control of different promoters. The one or more polypeptides in the third metabolic pathway can include a polypeptide having alcohol dehydrogenase activity, such as, for example an alcohol dehydrogenase (EC 1.1.1.1 class). Alcohol dehydrogenase are involved in the conversion of acetaldehyde and NADH into ethanol and NAD⁺. In some embodiments, the alcohol dehydrogenase is an iron-containing alcohol dehydrogenase. The alcohol dehydrogenase that can be expressed in the bacterial host cell includes, but is not limited to, ADH4 from Saccharomyces cerevisiae, ADHB from Zymonas mobilis, FUCO from Escherichia coli, ADHE from Escherichia coli, ADH1 from Clostridium acetobutylicum, ADH1 from Entamoeba nuttalli, BDHA from Clostridium acetobutylicum, BDHB from Clostridium acetobutylicum, 4HBD from Clostridium kluyveri, DHAT from Citrobacter freundii or DHAT from Klebsiella pneumoniae. In an embodiment, the alcohol dehydrogenase can be ADHB from Zymonas mobilis (Gene ID: AHJ71151.1), Lactobacillus reuteri (Accession Number: KRK51011.1), Lactobacillus mucosae (Accession Number WP_048345394.1), Lactobacillus brevis (Accession Number WP_003553163.1) or Streptococcus thermophiles (Accession Number WP_113870363.1). In an embodiment, the alcohol dehydrogenase can be from Lactiplantibacillus sp., such as, for example, from Lactiplantibacillus pentosus. In some embodiments, the alcohol dehydrogenase can have the amino acid of SEQ ID NO: 18, be a variant of SEQ ID NO: 18 (having alcohol dehydrogenase activity) or a fragment of SEQ ID NO: 18 (having alcohol dehydrogenase activity). In some embodiments, the alcohol dehydrogenase can have the amino acid of SEQ ID NO: 55, be a variant of SEQ ID NO: 55 (having alcohol dehydrogenase activity) or a fragment of SEQ ID NO: 55 (having alcohol dehydrogenase activity). In some embodiments, the alcohol dehydrogenase can have the amino acid of SEQ ID NO: 31, be a variant of SEQ ID NO: 31 (having alcohol dehydrogenase activity) or a fragment of SEQ ID NO: 31 (having alcohol dehydrogenase activity). In some embodiments, the alcohol dehydrogenase can have the amino acid of SEQ ID NO: 33, be a variant of SEQ ID NO: 33 (having alcohol dehydrogenase activity) or a fragment of SEQ ID NO: 33 (having alcohol dehydrogenase activity). In some specific embodiments, the alcohol dehydrogenase can be encoded by a heterologous nucleic acid molecule comprising the nucleic acid sequence of SEQ ID NO: 32 or 34, be a variant of the nucleic acid sequence of SEQ ID NO: 32 or 34 (encoding a polypeptide having alcohol dehydrogenase activity) or be a fragment of the nucleic acid sequence of SEQ ID NO: 32 or 34 (encoding a polypeptide having alcohol dehydrogenase activity). In yet another embodiment, the alcohol dehydrogenase can be encoded by heterologous nucleic acid molecule having a degenerate sequence encoding SEQ ID NO: 18, 31, 33 or 55.

In some embodiments, the bacterial host cell can also includes one or more genetic modification reducing the expression or inactivating one or more genes encoding one or more polypeptides in a pentose phosphate pathway. Without wishing to be bound to theory, the presence of such one or more genetic modification limits the production of fructose-6-phosphate and ultimately the accumulation of the fructose-1,6-bisphosphate, a key regulator of glycolytic flux. This reduction/inactivation can be achieved, for example, by deleting in part or totally the one or more genes encoding one or more polypeptides in a pentose phosphate pathway. This can also be achieved, for example, by introducing one or more nucleic acid residues in the opening reading frames of the one or more genes encoding one or more polypeptides in a pentose phosphate pathway. The inactivation can be made in one or all copies of the targeted gene. Genes of the pentose phosphate pathway includes a gene encoding a polypeptide having transketolase activity (a transketolase for example) as well as a gene encoding a polypeptide having transaldolase activity (a transaldose for example). In an embodiment, the bacterial host cell comprises a reduction in the activity or an inactivation in a gene encoding a polypeptide having transketolase activity, an ortholog thereof or a paralog thereof. In another embodiment, the bacterial host cell comprises a reduction in the activity or an inactivation in a gene encoding a polypeptide having transaldolase activity, an ortholog thereof or a paralog thereof. In still another embodiment, the bacterial host cell comprises a reduction in the activity or an inactivation in a gene encoding a polypeptide having transketolase activity (including orthlogs and paralogs thereof) and a gene encoding a polypeptide having transaldolase activity (including orthologs and paralogs thereof).

Carbon catabolite repression, e.g., the lack of ability of the bacterial cell to utilize a substrate such as glycerol when glucose is available, may be present in the recombinant bacterial host cell of the present disclosure. For example, carbon catabolite repression may be present in recombinant bacterial cells which were inoculated in a fermentation medium comprising more than 12.5 mM of glucose. In some embodiments, the recombinant bacterial host cell can be selected for its ability to exhibit low or no carbon catabolite repression and/or can be further modified to reduce or inactivate carbon catabolite repression. In such embodiments, the bacterial host cell may be able to utilize glycerol, even though the glucose concentration of fermentation medium is higher than 12.5 mM. Reduction or inactivation of catabolite repression can be achieved by introducing a further genetic modification in the bacterial host cell. For example, this further genetic modification can result in reducing the expression or inactivating at least one gene involved or causing carbon catabolite repression. In some embodiments, this can be achieved by reducing the expression or inactivating a gene whose promoter includes one or more catabolite response elements (cre). In Lactiplantibacillus, genes having at least one or more cre, include, but are not limited to, the malE (maltose-binding periplasmic protein precursor, treR (trehalose operon transcriptional repressor), tktAB (transketolase), gnd (6-phosphogluconate dehydrogenase), serS (serine-tRNA ligase), pox (pyruvate oxidase), epsH (glycosyltransferase EpsH), yodC (NAD(P)H nitroreductase), and yxeP (hydrolase) genes. Alternatively or in combination, this can be achieved, for example, by reducing the expression or inactivating at least a gene encoding a polypeptide of the phosphoenolpyruvate-dependent phosphotransferase system (PTS). In some embodiments, the polypeptide of the PTS is a transporter. In some additional embodiments, the PTS transporter is, for example, the mannose PTS transporter. When the recombinant bacterial is a lactic acid bacteria (such as, for example, from the Lactiplantibacillus sp. or from Lactococcus sp.), the mannose PTS transporter is referred to as EIIABCD^{mannose} and can be encoded by the manI/ABCD genes (also referred to as the manII operon). In some embodiments, the recombinant bacterial host cell of the present disclosure have a native phosphoenolpyruvate-dependent phosphotransferase system enzyme I gene (pstI) as well as a functional PtsI protein.

In some embodiments, the genetic modification for decreasing the expression or inactivating a gene involved in carbon catabolite repression can be coupled with another genetic modification of a gene encoding a polypeptide involved in the glycolytic flux. The genetic modification is intended to reduce the glycolytic flux in the bacterial host cell. In some embodiments, such additional genetic modification can be a reduction in the expression or a deletion in a gene encoding a glucose permease (such as GlcU, and in some embodiments GlcU2), a maltose PTS transporter (such as encoded by mapT only or in combination with the entire mapTPE operon), a maltose/maltodextrin transporter (such as the mdxEFG genes encoded by the mdx operon), a kinase (such as, for exampled a glucokinase (GlcK)), and/or a transcription factor (such as, for example, a transcriptional repressor like REX).

In some specific embodiments, the bacterial host cell comprises a plurality of genetic modifications to reduce the expression or inactivate the genes encoding mannose PTS transporter, glcU2, mapTPE, mdxEFG and REX.

In some embodiments, the bacterial host cell can be further modified to inactivate one or more endogenous genes. In a specific embodiment, the bacterial host cell can be modified to as to decrease its lactate dehydrogenase activity. As used in the context of the present disclosure, the expression “lactate dehydrogenase” refer to an enzyme of the E.C. 1.1.1.27 class which is capable of converting (e.g., catalyzing) the conversion of pyruvic acid into lactate. The bacterial host cells can thus have one or more gene coding for a polypeptide having lactate dehydrogenase activity which is inactivated (via partial or total deletion of the gene). In bacteria, the ldh1, ldh2, ldh3 and ldh4 genes encode polypeptides having lactate dehydrogenase activity. Some bacteria may contain as many as six or more such genes (i.e., ldh5, ldh6, etc.). In an embodiment, at least one of the ldh1, ldh2, ldh3 and ldh4 genes, their corresponding orthologs and paralogs is inactivated in the bacterial host cell. In an embodiment, only one of the ldh genes is inactivated in the bacterial host cell. For example, in the bacterial host cell of the present disclosure, only the ldh1 gene can be inactivated. In another embodiment, at least two of the ldh genes are inactivated in the bacterial host cell. In another embodiment, only two of the ldh genes are inactivated in the bacterial host cell. In a further embodiment, at least three of the ldh genes are inactivated in the bacterial host cell. In a further embodiment, only three of the ldh genes are inactivated in the bacterial host cell. In a further embodiment, at least four of the ldh genes are inactivated in the bacterial host cell. In a further embodiment, only four of the ldh genes are inactivated in the bacterial host cell. In a further embodiment, at least five of the ldh genes are inactivated in the bacterial host cell. In a further embodiment, only five of the ldh genes are inactivated in the bacterial host cell. In a further embodiment, at least six of the ldh genes are inactivated in the bacterial host cell. In a further embodiment, only six of the ldh genes are inactivated in the bacterial host cell. In still another embodiment, all of the ldh genes are inactivated in the bacterial host cell. Some bacteria may contain lactate dehydrogenase which are specific for the D- or L-enantiomer of lactate (i.e., D-ldh and L-ldh). In some embodiments, at least one D-ldh gene is inactivated in the bacterial host cell. In some embodiments, at least one L-ldh/gene is inactivated in the bacterial host cell. In additional embodiments, both the D-ldh and the L-ldh genes are inactivated in the bacterial host cell. In specific embodiments, the D-ldh1, L-ldh1 and D-ldh2 genere are inactivated in the bacterial host cell.

In some embodiments, the bacterial host cell, especially in embodiments in which the bacterial host cell is a lactic acid bacterium host cell, can express a bacteriocin. In some embodiments, the bacterial host cell can have the intrinsic ability (e.g., an ability that is not conferred by the introduction of a heterologous nucleic acid molecule) to express and produce at least one bacteriocin (e.g., a native bacteriocin). In some embodiments, the bacterial host cell can comprises one or more genetic modification to express and produce one or more bacteriocin (in addition to the one it already expresses, if any). In such embodiment, the bacterial host cell will include one or more heterologous nucleic acid molecule encoding the bacteriocin and/or the polypeptide(s) associated with the immunity to the bacteriocin. The coding sequence for the bacteriocin and for the polypeptide(s) associated with the immunity to the further bacteriocin can be provided on the same or distinct heterologous nucleic acid molecules. The heterologous nucleic acid molecule(s) (which can be heterologous) can be integrated in the bacterial chromosome or be independently replicating from the bacterial chromosome.

Bacteriocins are known as a class of peptides and polypeptides exhibiting, as their biological activity, anti-bacterial properties. Bacteriocins can exhibit bacteriostatic or cytotoxic activity. Bacteriocin can be provided as a monomeric polypeptide, a dimer polypeptide (homo- and heterodimers) as well as a circular polypeptide. Since bacteriocin are usually expressed to be exported outside of the cell, they are usually synthesized as pro-polypeptides including a leader sequence, the latter being cleaved upon secretion. The bacteriocin of the present disclosure can be expressed using their native leader sequence or a heterologous leader sequence. It is known in the art that some bacteriocins are modified after being translated to include uncommon amino acids (such as lanthionine, methyllanthionine, didehydroalanine, and/or didehydroaminobutyric acid). The amino acid sequences provided herein for the different bacteriocins do not include such post-translational modifications, but it is understood that a bacterial host cell expressing a bacteriocin from a second heterologous nucleic acid molecule can produce a polypeptide which does not exactly match the amino acid sequence of encoded by its corresponding gene, but the exported bacteriocin can be derived from such amino acid sequences (by post-translational modification).

In other embodiments, the bacterial host cell can also lack the intrinsic ability to express one or more bacteriocin and can be genetically modified to express and produce one or more bacteriocin (e.g., a recombinant bacteriocin). In such embodiment, the bacterial host cell can comprise one or more heterologous nucleic acid molecule encoding the recombinant bacteriocin and its associated immunity polypeptide(s). The coding sequence for the recombinant bacteriocin and for the polypeptide(s) associated with the immunity to the recombinant bacteriocin can be provided on the same or distinct nucleic acid molecules. In some embodiments, the bacterial host cell can be genetically modified to express and produce more than one recombinant bacteriocin and associated immunity polypeptide(s). In such embodiment, the bacterial host cell will include one or more heterologous nucleic acid molecule encoding the additional recombinant bacteriocin and/or the polypeptide(s) associated with the immunity to the additional recombinant bacteriocin. The coding sequence for the recombinant bacteriocin and for the polypeptide(s) associated with the immunity to the recombinant bacteriocin can be provided on the same or distinct nucleic acid molecules. The nucleic acid molecule(s) (which can be heterologous) can be integrated in the bacterial chromosome or be independently replicating from the bacterial chromosome.

In some embodiments, the bacterial host cell will be cultured in the presence of a bacteriocin it does not express (natively or in a recombinant fashion). For example, the biomass can be supplemented with a purified and exogenous source of a bacteriocin. In such embodiment, the bacterial host cell can be genetically modified to express and produce a polypeptide conferring immunity to the bacteriocin present in the biomass. In such embodiment, the bacterial host cell will include one or more heterologous nucleic acid molecule encoding a bacteriocin immunity polypeptide(s). When more than one type of bacteriocins are present in the biomass, the coding sequence for the polypeptide(s) associated with the immunity of each bacteriocin can be provided on the same or distinct nucleic acid molecules. In such embodiments, the bacterial host cell can be genetically modified to express and produce more than one associated bacteriocin immunity polypeptide. In such embodiment, the bacterial host cell will include one or more heterologous nucleic acid molecule encoding the additional polypeptide(s) associated with the immunity to each the bacteriocin present in the biomass. The coding sequence for the polypeptide(s) associated with the immunity to the bacteriocin(s) can be provided on the same or distinct nucleic acid molecules. Such heterologous nucleic acid molecule(s) can be integrated in the bacterial chromosome or be independently replicating from the bacterial chromosome.

In some embodiments, the at least one bacteriocin comprises one or more bacteriocin from Gram-negative bacteria. The bacteriocin from Gram-negative bacteria which can be used also or in combination with one or more additional bacteriocin. Bacteriocins from Gram-negative bacteria include, but are not limited to, microcins, colicin-like bacteriocins and tailocins. In some embodiments, the at least one bacteriocin comprises one or more bacteriocin from Gram-positive bacteria. The bacteriocin from Gram-positive bacteria which can be used also or in combination with one or more additional bacteriocin. Bacteriocins from Gram-positive bacteria include, but are not limited to, class I bacteriocins (such as, for example nisin A and/or nisin Z), class II bacteriocins, including class IIa (such as, for example, pediocin) and IIb (such as, for example, brochocin for example) bacteriocins, class III bacteriocins, class IV bacteriocins and circular bacteriocins (such as, for example, gassericin). Known bacteriocins include, but are not limited to, acidocin, actagardine, agrocin, alveicin, aureocin, aureocin A53, aureocin A70, bisin, carnocin, carnocyclin, caseicin, cerein, circularin A, colicin, curvaticin, divercin, duramycin, enterocin, enterolysin, epidermin/gallidermin, erwiniocin, gardimycin, gassericin A, glycinecin, halocin, haloduracin, klebicin, lactocin S, lactococcin, lacticin, leucoccin, lysostaphin, macedocin, mersacidin, mesentericin, microbisporicin, microcin S, mutacin, nisin A, nisin Z, paenibacillin, planosporicin, pediocin, pentocin, plantaricin, pneumocyclicin, pyocin, reutericin 6, sakaci, salivaricin, sublancin, subtilin, sulfolobicin, tasmancin, thuricin 17, trifolitoxin, variacin, vibriocin, warnericin and warnerin.

In a specific embodiment, the bacteriocin present in the biomass, expressed by the bacterial host cell or encoded by the heterologous nucleic acid molecule can be a Gram-positive class I bacteriocin. The Gram-positive class I bacteriocin can be the only bacteriocin expressed in the bacterial host cell or it can be expressed with one or more further bacteriocin. For example, nisin can be the only bacteriocin present in the biomass or produced by the bacterial host cell. In another example, nisin can be in combination with pediocin and brochocin in the biomass or expressed by the recombinant host bacterial cell. In some embodiments, the Gram-positive class I bacteriocin can be nisin A, nisin Z, nisin J, nisin H, nisin Q and/or nisin U. Nisin is a bacteriocin natively produced by some strains of Lactococcus lactis. Nisin is a relatively broad-spectrum bacteriocin effective against many Gram-positive organisms as well as spores.

In embodiments in which the bacterial host cell produces nisin as the bacteriocin or in which nisin is present in the biomass, the bacterial host cell can possess the machinery for making nisin or can be genetically engineered to express the machinery for making nisin. Polypeptides involved in the production and/or the regulation of production of nisin include, but are not limited to NisA, NisZ, NisJ, NisH, NisQ, NisB, NisT, NisC, NisP, NisR and/or NisK. The one or more polypeptides involved in the production and/or the regulation of production of nisin can be located on the same or a distinct nucleic acid molecule as the one encoding nisin.

In embodiments in which the bacterial host cell produces nisin as the bacteriocin or in which nisin is present in the biomass, the bacterial host cell possesses immunity against nisin or can be genetically engineered to gain immunity against nisin. A polypeptide known to confer immunity or resistance against nisin is NisI. Additional polypeptides involved in conferring immunity against nisin include, without limitation, NisE (which is a nisin transporter), NisF (which is a nisin transporter) and NisG (which is a nisin permease). As such, the second heterologous nucleic acid molecule can further encode NisE, NisF and/or NisG. The one or more polypeptides involved in the conferring immunity against nisin can be located on the same or on a distinct nucleic acid molecule as the one encoding nisin and/or the polypeptides involved in the production and/or the regulation of production of nisin.

In a specific embodiment, the bacteriocin present in the biomass or expressed by the bacterial host cell can be a Gram-positive class II bacteriocin. The Gram-positive class II bacteriocin can be the only bacteriocin expressed in the bacterial host cell or it can be expressed with one or more further bacteriocin. Gram-positive class II bacteriocins include two subgroups: class IIA and class IIB bacteriocins. In a specific example, the Gram-positive class IIA bacteriocin can be, without limitation, pediocin (also referred to as the PedA polypeptide).

In embodiments in which the bacterial host cell produces pediocin as the bacteriocin or in which pediocin is present in the biomass, the bacterial host cell can possess the machinery for making and regulating pediocin production or can be genetically engineered to express the machinery for making and regulating pediocin production. A polypeptide known to confer immunity or resistance against pediocin is PedB. As such, the bacterial host cell can express PedB or be genetically engineered to express PedB. In some embodiments, the heterologous nucleic acid molecule can further encode PedB (which can be present on the same nucleic acid molecule encoding PedA or a distinct one).

In a specific example, the Gram-positive class IIB bacteriocin can be, without limitation, brochocin. Brochocin is an heterodimer comprising a BrcA polypeptide and a BrcB polypeptide.

In embodiments in which the bacterial host cell produces brochocin as the bacteriocin or in which brochocin is present in the biomass, the bacterial host cell possesses immunity against brochocin. A polypeptide known to confer immunity or resistance against brochocin is BrcI. As such, the bacterial host cell can express BrcI or be genetically engineered to express BrcI. In some embodiments, the heterologous nucleic acid molecule can further encode BrcI (which can be present on the same nucleic acid molecule encoding BrcA/BrcB or a distinct one).

In embodiments in which the bacteriocin present in the biomass, expressed by the bacterial host cell is a Gram-positive class II bacteriocin, the bacterial host cell can express a native non-sec dependent secretory machinery and/or include one or more heterologous nucleic acid molecules encoding a native non-sec dependent secretory machinery for exporting the Gram-positive class II bacteriocin. An exemplary component of a non-sec dependent secretory machinery for exporting the Gram-positive class II bacteriocin is PedC (which can also be referred to as BrcD) which can have, in some additional embodiments, GenBank Accession Number WP_005918571, be a variant of GenBank Accession Number WP_005918571 having disulfide isomerase activity or be a fragment of GenBank Accession Number WP_005918571 having disulfide isomerase activity. A further exemplary component of a non-sec dependent secretory machinery for exporting the Gram-positive class II bacteriocin is PedD (which can also be referred to as PapD) which can have, in some additional embodiments, Uniprot Accession Number P36497.1, be a variant of Uniprot Accession Number P36497.1 having ATP-binding and transporting activity or be a fragment of Uniprot Accession Number P36497.1 having ATP-binding and transporting activity.

In some embodiments, the Gram-positive class II bacteriocin, its variants and its fragments can be associated with a sec-dependent leader peptide so as to facilitate its transport outside the bacterial host cell.

In a specific example, the Gram-positive cyclic bacteriocin can be gasserin. In such embodiment, the bacterial host cell is capable of expressing gasserin which can be expressed from the heterologous nucleic acid molecule.

In embodiments in which the bacterial host cell produces gasserin as the bacteriocin or in which gasserin is present in the culture medium, the bacterial host cell can possess the machinery for making or for regulating the production of gasserin or can be genetically engineered to express the machinery for making or for regulating the production of gasserin. Polypeptides involved in the machinery for making gasserin include, without limitations, GaaT (which is a gasserin transporter) and GaaE (which is a gasserin permease). As such, the heterologous nucleic acid molecule can further encode GaaT and/or GaaE (which can be on the same or on a different nucleic acid molecule than the one encoding gasserin).

In embodiments in which the bacterial host cell produces gasserin as the bacteriocin or in which gasserin is present in the biomass, the bacterial host cell possesses immunity against gasserin or can be genetically engineered to gain immunity against gasserin. A polypeptide known to confer immunity or resistance against gasserin is GaaI. As such, the heterologous nucleic acid molecule can further encode GaaI (which can be on the same or on a different nucleic acid molecule than the one encoding gasserin, GaaT or GaaE).

In embodiments in which the biomass comprises one or more antibiotic, it is important that the viability or the growth of the bacterial host cell is not reduced or slowed due to the presence of such antibiotic. As such, in some embodiments, the bacterial host cell can include one or more further nucleic acid molecule encoding one or more polypeptide involved in conferring resistance to the antibiotic(s) present in the biomass. Alternatively or in combination, the bacterial host cell can be made more resistant towards the antibiotic(s) present in the biomass by being submitted (prior to the fermentation) to an adaptation process. During an adaptation process, the bacterial host cell is submitted to increasing concentrations of the antibiotic for which resistance is sought.

In an embodiment, the bacterial host cell comprises one or more genes conferring resistance to a beta lactam, such as penicillin. In another embodiment, the bacterial host cell comprises one or more genes conferring resistance to streptogramin, such as virginiamycin. In another embodiment, the bacterial host cell comprises one or more genes conferring resistance to aminoglycoside, such as streptomycin. In yet a further embodiment, the bacterial host cell comprises one or more genes conferring resistance to a macrolide, such as, for example, erythromycin. In still another embodiment, the bacterial host cell comprises one or more genes conferring resistance to a polyether, such as monensin. In an embodiment, the bacterial host cell is adapted to become more resistant to a beta lactam, such as penicillin. In another embodiment, the bacterial host cell is adapted to become more resistant to streptogramin, such as virginiamycin. In another embodiment, the bacterial host cell com is adapted to become more resistant to aminoglycoside, such as streptomycin. In yet a further embodiment, the bacterial host cell is adapted to become more resistant to a macrolide, such as, for example, erythromycin. In still another embodiment, the bacterial host cell is adapted to become more resistant to a polyether, such as monensin.

The bacterial host cell described herein can be provided as a combination with the yeast cell described herein. In such combination, the bacterial host cell can be provided in a distinct container from the yeast cell. The bacterial host cell can be provided as a cell concentrate. The cell concentrate comprising the bacterial host cell can be obtained, for example, by propagating the bacterial host cells in a culture medium and removing at least one components of the medium comprising the propagated bacterial host cell. This can be done, for example, by dehydrating, filtering (including ultra-filtrating) and/or centrifuging the medium comprising the propagated bacterial host cell. In an embodiment, the bacterial host cell is provided as a frozen concentrate in the combination.

The bacterial host cell of the present disclosure can be provided in a composition comprising starch or a starch derivative. In some embodiments, the composition can also include a fermenting yeast or a yeast host cell.

In some embodiments, the bacterial host cell can be provided in a frozen form or a dried form (a lyophilized form for example).

### Fermenting Yeasts and Yeast Host Cell

The bacterial host cell of the present disclosure is used in combination with a recombinant yeast cell to convert the biomass into ethanol. In the context of the present disclosure, the recombinant yeast cell is considered to be a fermenting yeast cell because it is capable of converting the biomass into ethanol. The fermenting yeast cell can be a wild-type native yeast cell or a can be recombinant yeast host cell. In some embodiments, the yeasts can be provided from a population comprising both a wild-type native yeast cell and a recombinant yeast host cell.

Suitable fermenting yeasts and recombinant yeast host cells can be, for example, from the genus Saccharomyces, Kluyveromyces, Arxula, Debaryomyces, Candida, Pichia, Phaffia, Schizosaccharomyces, Hansenula, Kloeckera, Schwanniomyces or Yarrowia. Suitable yeast species can include, for example, S. cerevisiae, S. bulderi, S. barnetti, S. exiguus, S. uvarum, S. diastaticus, K. lactis, K. marxianus or K. fragilis. In some embodiments, the yeast is selected from the group consisting of Saccharomyces cerevisiae, Schizosaccharomyces pombe, Candida albicans, Pichia pastoris, Pichia stipitis, Yarrowia lipolytica, Hansenula polymorpha, Phaffia rhodozyma, Candida utilis, Arxula adeninivorans, Debaryomyces hansenii, Debaryomyces polymorphus, Schizosaccharomyces pombe and Schwanniomyces occidentalis. In one particular embodiment, the yeast is Saccharomyces cerevisiae. In some embodiments, the host cell can be an oleaginous yeast cell. For example, the oleaginous yeast host cell can be from the genus Blakeslea, Candida, Cryptococcus, Cunninghamella, Lipomyces, Mortierella, Mucor, Phycomyces, Pythium, Rhodosporidum, Rhodotorula, Trichosporon or Yarrowia. In some alternative embodiments, the host cell can be an oleaginous microalgae host cell (e.g., for example, from the genus Thraustochytrium or Schizochytrium). In an embodiment, the fermenting yeast or recombinant yeast host cell is from the genus Saccharomyces and, in some embodiments, from the species Saccharomyces cerevisiae.

The recombinant yeast host cell of the present disclosure has a metabolic pathway (referred to as the fourth metabolic pathway) comprising one or more (fourth) polypeptides for producing glycerol. The recombinant yeast host cell can have the intrinsic ability to produce glycerol (e.g., a native fourth metabolic pathway) and, in some embodiments, be selected based on this intrinsic ability. In some embodiments, the recombinant yeast host cell is capable, during a permissive fermentation of a corn mash to produce at least 0.25, 0.30, 0.35, 0.40, 0.45, 0.50, 0.55, 0.60, 0.65, 0.70, 0.75, 0.80, 0.85, 0.90, 1% or more of glycerol. Alternatively or in combination, the recombinant yeast host cell can be engineered to increase the activity in one or more fourth polypeptide in the fourth metabolic pathway (e.g., a heterologous fourth metabolic pathway). The increased in activity can be caused at least in part by introducing of one or more genetic modifications in a parental yeast host cell to obtain the recombinant yeast host cell. As such, the activity of the one or more fourth polypeptides of the recombinant yeast host cell is considered “increased” because it is higher than the activity of the one or more fourth polypeptides in the parental yeast host cell (e.g., prior to the introduction of the one or more genetic modifications). The one or more genetic modifications is not limited to a specific modification provided that it does increase the activity, and in some embodiments, the expression of the one or more fourth polypeptides and ultimately the production of glycerol. For example, the one or more genetic modifications can include the addition of a promoter to increase the expression of the one or more (native) fourth polypeptide. Alternatively or in addition, the one or more genetic modifications can include the introduction of one or more copies of a gene(s) encoding the one or more fourth (heterologous) polypeptides in the recombinant yeast host cell.

In some embodiments, the one or more fourth polypeptides for producing glycerol include, without limitation, a polypeptide having glycerol-3-phosphate dehydrogenase (GPD) activity and/or a polypeptide having glycerol-3-phosphate phosphatase (GPP) activity. In an embodiment, the yeast host cell comprises a polypeptide having glycerol-3-phosphate dehydrogenase activity. In another embodiment, the yeast host cell comprises a polypeptide having glycerol-3-phosphate phosphatase activity. In still another embodiment, the yeast host cell comprises a polypeptide having glycerol-3-phosphate dehydrogenase activity and a polypeptide having glycerol-3-phosphate phosphatase activity.

Polypeptides having glycerol-3-phosphate dehydrogenase activity include, without limitation, glycerol-3-phosphate dehydrogenases (E.C. Number 1.1.1.8) such as glycerol-3-phosphate dehydrogenase 1 (referred to as GPD1) and glycerol-3-phosphate dehydrogenase 2 (referred to as GPD2). The yeast host cell of the present disclosure can include (native or heterologous) GPD1, GPD2 or both.

Polypeptides having glycerol-3-phosphate phosphatase activity include, without limitation glycerol-3-phosphate phosphatases (E.C. Number 3.1.3.21) such as glycerol-3-phosphate phosphatase 1 (referred to GPP1) and glycerol-3-phosphate phosphatase 2 (GPP2). The yeast host cell of the present disclosure can include (native or heterologous) GPP1, GPP2 or both.

In yet another embodiment, the yeast host cell does not bear a genetic modification in its native genes for producing glycerol and includes its native genes coding for the GPP/GDP proteins.

The yeast host cell of the present disclosure can express the NAD-dependent glycerol-3-phosphate dehydrogenase GPD1 polypeptide or a GPD1 gene ortholog. GPD1 genes encoding the GPD1 polypeptide include, but are not limited to Saccharomyces cerevisiae Gene ID: 851539, Schizosaccharomyces pombe Gene ID: 2540547, Schizosaccharomyces pombe Gene ID: 2540455, Neurospora crassa Gene ID: 3873099, Candida albicans Gene ID: 3643924, Scheffersomyces stipitis Gene ID: 4840320, Spathaspora passalidarum Gene ID: 18874668, Trichoderma reesei Gene ID: 18482691, Nectria haematococca Gene ID: 9668637, Candida dubliniensis Gene ID: 8046432, Chlamydomonas reinhardtii Gene ID: 5716580, Brassica napus Gene ID: 106365675, Chlorella variabilis Gene ID: 17355036, Brassica napus Gene ID: 106352802, Mus musculus Gene ID: 14555, Homo sapiens Gene ID: 2819, Rattus norvegicus Gene ID: 60666, Sus scrofa Gene ID: 100153250, Gallus gallus Gene ID: 426881, Bos taurus Gene ID: 525042, Xenopus tropicalis Gene ID: 448519, Pan troglodytes Gene ID: 741054, Canis lupus familiaris Gene ID: 607942, Callorhinchus milii Gene ID: 103188923, Columba livia Gene ID: 102088900, Macaca fascicularis Gene ID: 101865501, Myotis brandtii Gene ID: 102257341, Heterocephalus glaber Gene ID: 101702723, Nannospalax galili Gene ID: 103746543, Mustela putorius furo Gene ID: 101681348, Callithrix jacchus Gene ID: 100414900, Labrus bergylta Gene ID: 109980872, Monopterus albus Gene ID: 109969143, Castor canadensis Gene ID: 109695417, Paralichthys olivaceus Gene ID: 109635348, Bos indicus Gene ID: 109559120, Hippocampus comes Gene ID: 109507993, Rhinolophus sinicus Gene ID: 109443801, Hipposideros armiger Gene ID: 109393253, Crocodylus porosus Gene ID: 109324424, Gavialis gangeticus Gene ID: 109293349, Panthera pardus Gene ID: 109249099, Cyprinus carpio Gene ID: 109094445, Scleropages formosus Gene ID: 108931403, Nanorana parkeri Gene ID: 108789981, Rhinopithecus bieti Gene ID: 108543924, Lepidothrix coronata Gene ID: 108509436, Pygocentrus nattereri Gene ID: 108444060, Manis javanica Gene ID: 108406536, Cebus capucinus imitator Gene ID: 108316082, Ictalurus punctatus Gene ID: 108255083, Kryptolebias marmoratus Gene ID: 108231479, Miniopterus natalensis Gene ID: 107528262, Rousettus aegyptiacus Gene ID: 107514265, Coturnix japonica Gene ID: 107325705, Protobothrops mucrosquamatus Gene ID: 107302714, Parus major Gene ID: 107215690, Marmota marmota marmota Gene ID: 107148619, Gekko japonicus Gene ID: 107122513, Cyprinodon variegatus Gene ID: 107101128, Acinonyx jubatus Gene ID: 106969233, Poecilia latipinna Gene ID: 106959529, Poecilia mexicana Gene ID: 106929022, Calidris pugnax Gene ID: 106891167, Sturnus vulgaris Gene ID: 106863139, Equus asinus Gene ID: 106845052, Thamnophis sirtalis Gene ID: 106545289, Apteryx australis mantelli Gene ID: 106499434, Anser cygnoides domesticus Gene ID: 106047703, Dipodomys ordii Gene ID: 105987539, Clupea harengus Gene ID: 105897935, Microcebus murinus Gene ID: 105869862, Propithecus coquereli Gene ID: 105818148, Aotus nancymaae Gene ID: 105709449, Cercocebus atys Gene ID: 105580359, Mandrillus leucophaeus Gene ID: 105527974, Colobus angolensis palliatus Gene ID: 105507602, Macaca nemestrina Gene ID: 105492851, Aquila chrysaetos canadensis Gene ID: 105414064, Pteropus vampyrus Gene ID: 105297559, Camelus dromedarius Gene ID: 105097186, Camelus bactrianus Gene ID: 105076223, Esox lucius Gene ID: 105016698, Bison bison bison Gene ID: 105001494, Notothenia coriiceps Gene ID: 104967388, Larimichthys crocea Gene ID: 104928374, Fukomys damarensis Gene ID: 04861981, Haliaeetus leucocephalus Gene ID: 104831135, Corvus cornix cornix Gene ID: 104683744, Rhinopithecus roxellana Gene ID: 104679694, Balearica regulorum gibbericeps Gene ID: 104630128, Tinamus guttatus Gene ID: 104575187, Mesitornis unicolor Gene ID: 104539793, Antrostomus carolinensis Gene ID: 104532747, Buceros rhinoceros silvestris Gene ID: 104501599, Chaetura pelagica Gene ID: 104385595, Leptosomus discolor Gene ID: 104353902, Opisthocomus hoazin Gene ID: 104326607, Charadrius vociferus Gene ID: 104284804, Struthio camelus australis Gene ID: 104144034, Egretta garzetta Gene ID: 104132778, Cuculus canorus Gene ID: 104055090, Nipponia nippon Gene ID: 104011969, Pygoscelis adeliae Gene ID: 103914601, Aptenodytes forsteri Gene ID: 103894920, Serinus canaria Gene ID: 103823858, Manacus vitellinus Gene ID: 103760593, Ursus maritimus Gene ID: 103675473, Corvus brachyrhynchos Gene ID: 103613218, Galeopterus variegatus Gene ID: 103598969, Equus przewalskii Gene ID: 103546083, Calypte anna Gene ID: 103536440, Poecilia reticulata Gene ID: 103464660, Cynoglossus semilaevis Gene ID: 103386748, Stegastes partitus Gene ID: 103355454, Eptesicus fuscus Gene ID: 103285288, Chlorocebus sabaeus Gene ID: 103238296, Orycteropus afer afer Gene ID: 103194426, Poecilia formosa Gene ID: 103134553, Erinaceus europaeus Gene ID: 103118279, Lipotes vexillifer Gene ID: 103087725, Python bivittatus Gene ID: 103049416, Astyanax mexicanus Gene ID: 103021315, Balaenoptera acutorostrata scammoni Gene ID: 103006680, Physeter catodon Gene ID: 102996836, Panthera tigris altaica Gene ID: 102961238, Chelonia mydas Gene ID: 102939076, Peromyscus maniculatus bairdii Gene ID: 102922332, Pteropus alecto Gene ID: 102880604, Elephantulus edwardii Gene ID: 102844587, Chrysochloris asiatica Gene ID: 102825902, Myotis davidii Gene ID: 102754955, Leptonychotes weddellii Gene ID: 102730427, Lepisosteus oculatus Gene ID: 102692130, Alligator mississippiensis Gene ID: 102576126, Vicugna pacos Gene ID: 102542115, Camelus ferus Gene ID: 102507052, Tupaia chinensis Gene ID: 102482961, Pelodiscus sinensis Gene ID: 102446147, Myotis lucifugus Gene ID: 102420239, Bubalus bubalis Gene ID: 102395827, Alligator sinensis Gene ID: 102383307, Latimeria chalumnae Gene ID: 102345318, Pantholops hodgsonii Gene ID: 102326635, Haplochromis burtoni Gene ID: 102295539, Bos mutus Gene ID: 102267392, Xiphophorus maculatus Gene ID: 102228568, Pundamilia nyererei Gene ID: 102192578, Capra hircus Gene ID: 102171407, Pseudopodoces humilis Gene ID: 102106269, Zonotrichia albicollis Gene ID: 102070144, Falco cherrug Gene ID: 102047785, Geospiza fortis Gene ID: 102037409, Chinchilla lanigera Gene ID: 102014610, Microtus ochrogaster Gene ID: 101990242, Ictidomys tridecemlineatus Gene ID: 101955193, Chrysemys picta Gene ID: 101939497, Falco peregrinus Gene ID: 101911770, Mesocricetus auratus Gene ID: 101824509, Ficedula albicollis Gene ID: 101814000, Anas platyrhynchos Gene ID: 101789855, Echinops telfairi Gene ID: 101641551, Condylura cristata Gene ID: 101622847, Jaculus jaculus Gene ID: 101609219, Octodon degus Gene ID: 101563150, Sorex araneus Gene ID: 101556310, Ochotona princeps Gene ID: 101532015, Maylandia zebra Gene ID: 101478751, Dasypus novemcinctus Gene ID: 101446993, Odobenus rosmarus divergens Gene ID: 101385499, Tursiops truncatus Gene ID: 101318662, Orcinus orca Gene ID: 101284095, Oryzias latipes Gene ID: 101154943, Gorilla gorilla Gene ID: 101131184, Ovis aries Gene ID: 101119894, Felis catus Gene ID: 101086577, Takifugu rubripes Gene ID: 101079539, Saimiri boliviensis Gene ID: 101030263, Papio anubis Gene ID: 101004942, Pan paniscus Gene ID: 100981359, Otolemur garnettii Gene ID: 100946205, Sarcophilus harrisii Gene ID: 100928054, Cricetulus griseus Gene ID: 100772179, Cavia porcellus Gene ID: 100720368, Oreochromis niloticus Gene ID: 100712149, Loxodonta africana Gene ID: 100660074, Nomascus leucogenys Gene ID: 100594138, Anolis carolinensis Gene ID: 100552972, Meleagris gallopavo Gene ID: 100542199, Ailuropoda melanoleuca Gene ID: 100473892, Oryctolagus cuniculus Gene ID: 100339469, Taeniopygia guttata Gene ID: 100225600, Pongo abelii Gene ID: 100172201, Ornithorhynchus anatinus Gene ID: 100085954, Equus caballus Gene ID: 100052204, Mus musculus Gene ID: 100198, Xenopus laevis Gene ID: 399227, Danio rerio Gene ID: 325181, Danio rerio Gene ID: 406615, Melopsittacus undulatus Gene ID: 101872435, Ceratotherium simum simum Gene ID: 101408813, Trichechus manatus latirostris Gene ID: 101359849 and Takifugu rubripes Gene ID: 101071719).

The yeast host cells of the present disclosure can express the NAD-dependent glycerol-3-phosphate dehydrogenase GPD2 polypeptide or a GPD2 gene ortholog. GPD2 genes encoding the GPD2 polypeptide include, but are not limited to Mus musculus Gene ID: 14571, Homo sapiens Gene ID: 2820, Saccharomyces cerevisiae Gene ID: 854095, Rattus norvegicus Gene ID: 25062, Schizosaccharomyces pombe Gene ID: 2541502, Mus musculus Gene ID: 14380, Danio rerio Gene ID: 751628, Caenorhabditis elegans Gene ID: 3565504, Mesocricetus auratus Gene ID: 101825992, Xenopus tropicalis Gene ID: 779615, Macaca mulatta Gene ID: 697192, Bos taurus Gene ID: 504948, Canis lupus familiaris Gene ID: 478755, Cavia porcellus Gene ID: 100721200, Gallus gallus Gene ID: 424321, Pan troglodytes Gene ID: 459670, Oryctolagus cuniculus Gene ID: 100101571, Candida albicans Gene ID: 3644563, Xenopus laevis Gene ID: 444438, Macaca fascicularis Gene ID: 102127260, Ailuropoda melanoleuca Gene ID: 100482626, Cricetulus griseus Gene ID: 100766128, Heterocephalus glaber Gene ID: 101715967, Scheffersomyces stipitis Gene ID: 4838862, Ictalurus punctatus Gene ID: 108273160, Mustela putorius furo Gene ID: 101681209, Nannospalax galili Gene ID: 103741048, Callithrix jacchus Gene ID: 100409379, Lates calcarifer Gene ID: 108873068, Nothobranchius furzeri Gene ID: 07384696, Acanthisitta chloris Gene ID: 103808746, Acinonyx jubatus Gene ID: 106978985, Alligator mississippiensis Gene ID: 102562563, Alligator sinensis Gene ID: 102380394, Anas platyrhynchos, Anolis carolinensis Gene ID: 100551888, Anser cygnoides domesticus Gene ID: 106043902, Aotus nancymaae Gene ID: 105719012, Apaloderma vittatum Gene ID: 104281080, Aptenodytes forsteri Gene ID: 103893867, Apteryx australis mantelli Gene ID: 106486554, Aquila chrysaetos canadensis Gene ID: 105412526, Astyanax mexicanus Gene ID: 103029081, Austrofundulus limnaeus Gene ID: 106535816, Balaenoptera acutorostrata scammoni Gene ID: 103019768, Balearica regulorum gibbericeps, Bison bison bison Gene ID: 104988636, Bos indicus Gene ID: 109567519, Bos mutus Gene ID: 102277350, Bubalus bubalis Gene ID: 102404879, Buceros rhinoceros silvestris Gene ID: 104497001, Calidris pugnax Gene ID: 106902763, Callorhinchus milii Gene ID: 103176409, Calypte anna Gene ID: 103535222, Camelus bactrianus Gene ID: 105081921, Camelus dromedarius Gene ID: 105093713, Camelus ferus Gene ID: 102519983, Capra hircus Gene ID: 102176370, Cariama cristata Gene ID: 104154548, Castor canadensis Gene ID: 109700730, Cebus capucinus imitator Gene ID: 108316996, Cercocebus atys Gene ID: 105576003, Chaetura pelagica Gene ID: 104391744, Charadrius vociferus Gene ID: 104286830, Chelonia mydas Gene ID: 102930483, Chinchilla lanigera Gene ID: 102017931, Chlamydotis macqueenii Gene ID: 104476789, Chlorocebus sabaeus Gene ID: 103217126, Chrysemys picta Gene ID: 101939831, Chrysochloris asiatica Gene ID: 102831540, Clupea harengus Gene ID: 105902648, Colius striatus Gene ID: 104549356, Colobus angolensis palliatus Gene ID: 105516852, Columba livia Gene ID: 102090265, Condylura cristata Gene ID: 101619970, Corvus brachyrhynchos, Coturnix japonica Gene ID: 107316969, Crocodylus porosus Gene ID: 109322895, Cuculus canorus Gene ID: 104056187, Cynoglossus semilaevis Gene ID: 103389593, Dasypus novemcinctus Gene ID: 101428842, Dipodomys ordii Gene ID: 105996090, Echinops telfairi Gene ID: 101656272, Egretta garzetta Gene ID: 104135263, Elephantulus edwardii Gene ID: 102858276, Eptesicus fuscus Gene ID: 103283396, Equus asinus Gene ID: 106841969, Equus caballus Gene ID: 100050747, Equus przewalskii Gene ID: 103558835, Erinaceus europaeus Gene ID: 103114599, Eurypyga helias Gene ID: 104502666, Falco cherrug Gene ID: 102054715, Falco peregrinus Gene ID: 101912742, Felis catus Gene ID: 101089953, Ficedula albicollis Gene ID: 101816901, Fukomys damarensis Gene ID: 104850054, Fundulus heteroclitus Gene ID: 105936523, Galeopterus variegatus Gene ID: 103586331, Gavia stellata Gene ID: 104250365, Gavialis gangeticus Gene ID: 109301301, Gekko japonicus Gene ID: 107110762, Geospiza fortis Gene ID: 102042095, Gorilla gorilla Gene ID: 101150526, Haliaeetus albicilla Gene ID: 104323154, Haliaeetus leucocephalus Gene ID: 104829038, Haplochromis burtoni Gene ID: 102309478, Hippocampus comes Gene ID: 109528375, Hipposideros armiger Gene ID: 109379867, Ictidomys tridecemlineatus Gene ID: 101965668, Jaculus jaculus Gene ID: 101616184, Kryptolebias marmoratus Gene ID: 108251075, Labrus bergylta Gene ID: 109984158, Larimichthys crocea Gene ID: 104929094, Latimeria chalumnae Gene ID: 102361446, Lepidothrix coronata Gene ID: 108501660, Lepisosteus oculatus Gene ID: 102691231, Leptonychotes weddellii Gene ID: 102739068, Leptosomus discolor Gene ID: 104340644, Lipotes vexillifer Gene ID: 103074004, Loxodonta africana Gene ID: 100654953, Macaca nemestrina Gene ID: 105493221, Manacus vitellinus Gene ID: 103757091, Mandrillus leucophaeus Gene ID: 105548063, Manis javanica Gene ID: 108392571, Marmota marmota marmota Gene ID: 107136866, Maylandia zebra Gene ID: 101487556, Mesitornis unicolor Gene ID: 104545943, Microcebus murinus Gene ID: 105859136, Microtus ochrogaster Gene ID: 101999389, Miniopterus natalensis Gene ID: 107525674, Monodelphis domestica Gene ID: 100014779, Monopterus albus Gene ID: 109957085, Myotis brandtii Gene ID: 102239648, Myotis davidii Gene ID: 102770109, Myotis lucifugus Gene ID: 102438522, Nanorana parkeri Gene ID: 108784354, Nestor notabilis Gene ID: 104399051, Nipponia nippon Gene ID: 104012349, Nomascus leucogenys Gene ID: 100590527, Notothenia coriiceps Gene ID: 104964156, Ochotona princeps Gene ID: 101530736, Octodon degus Gene ID: 101591628, Odobenus rosmarus divergens Gene ID: 101385453, Oncorhynchus kisutch Gene ID: 109870627, Opisthocomus hoazin Gene ID: 104338567, Orcinus orca Gene ID: 101287409, Oreochromis niloticus Gene ID: 100694147, Ornithorhynchus anatinus Gene ID: 100081433, Orycteropus afer afer Gene ID: 103197834, Oryzias latipes Gene ID: 101167020, Otolemur garnettii Gene ID: 100966064, Ovis aries Gene ID: 443090, Pan paniscus Gene ID: 100970779, Panthera pardus Gene ID: 109271431, Panthera tigris altaica Gene ID: 102957949, Pantholops hodgsonii Gene ID: 102323478, Papio anubis Gene ID: 101002517, Paralichthys olivaceus Gene ID: 109631046, Pelodiscus sinensis Gene ID: 102454304, Peromyscus maniculatus bairdii Gene ID: 102924185, Phaethon lepturus Gene ID: 104624271, Phalacrocorax carbo Gene ID: 104049388, Physeter catodon Gene ID: 102978831, Picoides pubescens Gene ID: 104296936, Poecilia latipinna Gene ID: 106958025, Poecilia mexicana Gene ID: 106920534, Poecilia reticulata Gene ID: 103473778, Pongo abelii Gene ID: 100452414, Propithecus coquereli Gene ID: 105807399, Protobothrops mucrosquamatus Gene ID: 107289584, Pseudopodoces humilis Gene ID: 102109711, Pterocles gutturalis Gene ID: 104461236, Pteropus alecto Gene ID: 102879110, Pteropus vampyrus Gene ID: 105291402, Pundamilia nyererei Gene ID: 102200268, Pygocentrus nattereri Gene ID: 108411786, Pygoscelis adeliae Gene ID: 103925329, Python bivittatus Gene ID: 103059167, Rhincodon typus Gene ID: 109920450, Rhinolophus sinicus Gene ID: 109445137, Rhinopithecus bieti Gene ID: 108538766, Rhinopithecus roxellana Gene ID: 104654108, Rousettus aegyptiacus Gene ID: 107513424, Saimiri boliviensis Gene ID: 101027702, Salmo salar Gene ID: 106581822, Sarcophilus harrisii Gene ID: 100927498, Scleropages formosus Gene ID: 108927961, Serinus canaria Gene ID: 103814246, Sinocyclocheilus grahami Gene ID: 107555436, Sorex araneus Gene ID: 101543025, Stegastes partitus Gene ID: 103360018, Struthio camelus australis Gene ID: 104138752, Sturnus vulgaris Gene ID: 106861926, Sugiyamaella lignohabitans Gene ID: 30033324, Sus scrofa Gene ID: 397348, Taeniopygia guttata Gene ID: 100222867, Takifugu rubripes Gene ID: 101062218, Tarsius syrichta Gene ID: 103254049, Tauraco erythrolophus Gene ID: 104378162, Thamnophis sirtalis Gene ID: 106538827, Tinamus guttatus Gene ID: 104572349, Tupaia chinensis Gene ID: 102471148, Tursiops truncatus Gene ID: 101330605, Ursus maritimus Gene ID: 103659477, Vicugna pacos Gene ID: 102533941, Xiphophorus maculatus Gene ID: 102225536, Zonotrichia albicollis Gene ID: 102073261, Ciona intestinalis Gene ID: 100183886, Meleagris gallopavo Gene ID: 100546408, Trichechus manatus latirostris Gene ID: 101355771, Ceratotherium simum simum Gene ID: 101400784, Melopsittacus undulatus Gene ID: 101871704, Esox lucius Gene ID: 10502249 and Pygocentrus nattereri Gene ID: 108411786. In an embodiment, the GPD2 polypeptide is encoded by Saccharomyces cerevisiae Gene ID: 854095.

The yeast host cell of the present disclosure can express the glycerol-1-phosphatase 1 (GPP1) polypeptide or a GPP1 gene ortholog/paralog. GPP1 genes encoding the GPP1 polypeptide include, but are not limited to Saccharomyces cerevisiae Gene ID: 854758, Arabidopsis thaliana Gene ID: 828690, Scheffersomyces stipitis Gene ID: 4836794, Chlorella variabilis Gene ID: 17352997, Solanum tuberosum Gene ID: 102585195, Homo sapiens Gene ID: 7316, Millerozyma farinosa Gene ID: 14521241, 14520178, 1451927 and 14518181, Sugiyamaella lignohabitans Gene ID: 30035078, Candida dubliniensis Gene ID: 8046759.

The yeast host cell of the present disclosure can express the glycerol-1-phosphatase GPP2 polypeptide or a GPP2 gene ortholog/paralog. GPP2 genes encoding the GPP2 polypeptide include, but are not limited to Saccharomyces cerevisiae Gene ID: 856791, Sugiyamaella lignohabitans Gene ID: 30035078, Arabidopsis thaliana Gene ID: 835849, Nicotiana attenuata Gene ID: 109234217, Candida albicans Gene ID: 3640236, Candida glabrata Gene ID: 2891433, 2891243 and 2889223.

In some embodiments, the recombinant yeast host cell can include a reduction in activity or an inactivation in one or more genes encoding one or more polypeptides for producing glycerol. In some embodiments, the recombinant yeast host cell that has been engineered to include a reduction in activity or an inactivation is capable, during a permissive fermentation of a corn mash to produce at least 0.25, 0.30, 0.35, 0.40, 0.45, 0.50, 0.55, 0.60, 0.65, 0.70, 0.75, 0.80, 0.85, 0.90, 1% or more of glycerol. The reduction in activity or the inactivation can be engineered in one or more genes encoding one or more polypeptides for producing glycerol. In the context of the present disclosure, the recombinant yeast host cell does not include an inactivation in both GPD1 and GPD2.

Optionally, the recombinant yeast host cell can also include a reduction in activity or an inactivation in one or more genes encoding one or more polypeptides capable of catabolizing glycerol. This features favors the accumulation of glycerol for utilization by the bacterial host cell. Polypeptides capable of catabolizing glycerol include, without limitation, a polypeptide having glycerol dehydrogenase activity (a glycerol dehydrogenase for example) and/or a polypeptide having dihydroxyacetone kinase activity (a dihydroxyacetone kinase for example). Therefore, the recombinant yeast host cell of the present disclosure can include a genetic modification to reduce the expression of or inactivate a gene encoding a polypeptide having glycerol dehydrogenase activity (a gene encoding a glycerol dehydrogenase for example), an ortholog thereof or a paralog thereof. The recombinant yeast host cell of the present disclosure can include a genetic modification to reduce the expression of or inactivate a gene encoding a polypeptide having dihydroxyacetone kinase activity (a gene encoding a dihydroxyacetone kinase for example), an ortholog thereof or a paralog thereof. The recombinant yeast host cell of the present disclosure can include a genetic modification to reduce the expression of or inactivate a gene encoding a polypeptide having glycerol dehydrogenase activity and of a gene encoding a polypeptide having dihydroxyacetone kinase activity.

In some embodiments, the recombinant yeast host cell can have a genetic modification for increasing the activity of one or more native and/or heterologous polypeptides to limit the export of glycerol outside the cell or favor import glycerol inside the recombinant yeast host cell. This can be achieved, for example, by reducing the activity (and in some embodiment inactivating) of a polypeptide involved in the export of glycerol (FPS1 for example) and/or by increasing the activity of a polypeptide involved in the import of glycerol (STL1 for example). In some embodiments, the recombinant yeast host cell that has been engineered is capable, during a permissive fermentation of a corn mash to produce at least 0.25, 0.30, 0.35, 0.40, 0.45, 0.50, 0.55, 0.60, 0.65, 0.70, 0.75, 0.80, 0.85, 0.90, 1% or more of glycerol. For example, when the recombinant yeast host cell is engineered to increase the activity of a polypeptide involved in the importation of glycerol, the genetic modification can comprise including a heterologous promoter which increases the expression (and ultimately the activity) of the native polypeptide capable of importing glycerol. In still another example, the genetic recombination can cause a mutation in the coding sequence of the polypeptide that function to import glycerol which increases the activity of the mutated polypeptide (when compared to the native polypeptide). In yet another example, in an embodiment in which the one or more protein is a heterologous protein, the genetic modification can comprise introducing one or more copies of a heterologous nucleic acid molecule to increase the expression (and ultimately the activity) of the heterologous polypeptide to increase the import of glycerol.

An exemplary polypeptide capable of functioning to import glycerol is the glucose-inactivated glycerol/proton symporter STL1. The native function of the STL1 polypeptide is the uptake of glycerol from the extracellular environment. STL1 is a member of the Sugar Porter Family which is part of the Major Facilitator Superfamily (MFS). STL1 transports glycerol by proton symport meaning that the glycerol and protons are cotransported through STL1 into the cell. In S. cerevisiae, STL1's expression and glycerol uptake is typically repressed when carbon sources such as glucose are available. When the cells undergo high osmotic shock, STL1 is expressed in order to help deal with the osmotic shock by transporting the osmoprotectant glycerol into the cell and increasing the intracellular glycerol concentration. In the context of the present disclosure, the protein functioning to import glycerol can be the STL1 polypeptide, a variant of the STL1 polypeptide, a fragment of the STL1 polypeptide or a polypeptide encoded by a STL1 gene ortholog/paralog.

The heterologous polypeptide functioning to import glycerol can be encoded by a STL1 gene. The STL1 polypeptide is natively expressed in yeasts and fungi, therefore the heterologous protein functioning to import glycerol can be derived from yeasts and fungi. STL1 genes encoding the STL1 protein include, but are not limited to, Saccharomyces cerevisiae Gene ID: 852149, Candida albicans Gene ID 3703976, Kluyveromyces lactis Gene ID: 2896463, Ashbya gossypii Gene ID: 4620396, Eremothecium sinecaudum Gene ID: 28724161, Torulaspora delbrueckii Gene ID: 11505245, Lachancea thermotolerans Gene ID: 8290820, Phialophora attae Gene ID: 28742143, Penicillium digitatum Gene ID: 26229435, Aspergillus oryzae Gene ID: 5997623, Aspergillus fumigatus Gene ID: 3504696, Talaromyces atroroseus Gene ID: 31007540, Rasamsonia emersonii Gene ID: 25315795, Aspergillus flavus Gene ID: 7910112, Aspergillus terreus Gene ID: 4322759, Penicillium chrysogenum Gene ID: 8310605, Alternaria alternata Gene ID: 29120952, Paraphaeosphaeria sporulosa Gene ID: 28767590, Pyrenophora tritici-repentis Gene ID: 6350281, Metarhizium robertsii Gene ID: 19259252, Isaria fumosorosea Gene ID: 30023973, Cordyceps militaris Gene ID: 18171218, Pochonia chlamydosporia Gene ID: 28856912, Metarhizium majus Gene ID: 26274087, Neofusicoccum parvum Gene ID: 19029314, Diplodia corticola Gene ID: 31017281, Verticillium dahliae Gene ID: 20711921, Colletotrichum gloeosporioides Gene ID: 18740172, Verticillium albo-atrum Gene ID: 9537052, Paracoccidioides lutzii Gene ID: 9094964, Trichophyton rubrum Gene ID: 10373998, Nannizzia gypsea Gene ID: 10032882, Trichophyton verrucosum Gene ID: 9577427, Arthroderma benhamiae Gene ID: 9523991, Magnaporthe oryzae Gene ID: 2678012, Gaeumannomyces graminis var. tritici Gene ID: 20349750, Togninia minima Gene ID: 19329524, Eutypa lata Gene ID: 19232829, Scedosporium apiospermum Gene ID: 27721841, Aureobasidium namibiae Gene ID: 25414329, Sphaerulina musiva Gene ID: 27905328 as well as Pachysolen tannophilus GenBank Accession Numbers JQ481633 and JQ481634, Saccharomyces paradoxus STL1 (and can have, for example, the amino acid sequence of SEQ ID NO: 57, be a variant thereof or a fragment thereof) and Millerozyma farinosa (and can have, for example, the amino acid sequence of SEQ ID NO: 58, be a variant thereof or a fragment thereof). In an embodiment, the STL1 protein is encoded by Saccharomyces cerevisiae Gene ID: 852149 and can have, for example, the amino acid sequence of SEQ ID NO: 59 (a variant thereof or a fragment thereof).

The FPS1 polypeptide is an exemplary polypeptide which functions to export glycerol. The FPS1 polypeptide is a channel protein located in the plasma membrane that controls the accumulation and release of glycerol in yeast osmoregulation. As such, the modification can include reducing or inactivating the expression of the gene encoding the FPS1 polypeptide, optionally during glycolytic conditions.

In the context of the present disclosure, the recombinant yeast host cell has a (fifth) metabolic pathway for generating acetate. The metabolic pathway for generating acetate comprises one or more fifth polypeptides for generating acetate. In some embodiments, the yeast host cell of the present disclosure is capable to produce at least 10.0, 12.5, 15.0, 17.5, 20.0, 22.5, 25.0, 27.5, 30.0, 32.5, 35.0, 37.5, 40.0, 42.5, 45.0, 47.5, 50 mM or more of acetate during a permissive fermentation of a corn mash. In an embodiment, the one or more fifth polypeptides comprises a polypeptide having phosphoketolase activity. The yeast host cell can be engineered to provide or increase its phosphoketolase activity (e.g., a heterologous phosphoketolase activity). When the phosphoketolase activity is engineered, the increased in phosphoketolase activity can be caused at least in part by introducing of one or more genetic modifications in a native yeast host cell to obtain the recombinant yeast host cell. In an example, the phosphoketolase activity of the recombinant yeast host cell is considered “increased” because it is higher than the phosphoketolase activity of the native yeast host cell (e.g., prior to the introduction of the one or more genetic modifications). The one or more genetic modifications are not limited to a specific modification provided that it does increase phosphoketolase activity. For example, the one or more genetic modifications can include the addition of a promoter to increase the expression of the one or more (native) first polypeptides having phosphoketolase activity. Alternatively or in addition, the one or more genetic modifications can include the introduction of one or more copies of a gene encoding the one or more first (heterologous) polypeptide having phosphoketolase activity in the recombinant yeast host cell.

As used in the context of the present disclosure, a polypeptide having phosphoketolase activity is capable of converting (e.g., catalyzing) xylulose-5-phosphate (and in some embodiments fructose-6-phosphate) into acetyl phosphate, D-glyceraldehyde 3-phosphate and water (E.C. 4.1.2.9 and 4.1.2.22). The yeast host cell of the present disclosure can include a native or a heterologous polypeptide having phosphoketolase activity (a phosphoketolase for example). In some embodiments, the polypeptide having phosphoketolase activity is a single-specificity phosphoketolase (e.g., it catabolizes either xylulose-5-phosphate or fructose-6-phosphate). In some embodiments, the polypeptide having phosphoketolase activity is a dual-specificity phosphoketolase (e.g., it can catabolize xylulose 5-phosphate and fructose-6-phosphate). In some embodiments, the polypeptide having phosphoketolase activity can also exhibit phosphatase activity. In some embodiments, the phosphoketolase (PHK) is derived from a genus selected from the group consisting of Aspergillus, Neurospora, Lactobacillus, Lactiplantibacillus, Bifidobacterium, Leuconostoc, Oenococcus, and Penicillium. In some embodiments, the PHK is from Bifidobacterium adolescentis (and can have, for example, the amino acid sequence of SEQ ID NO: 1, be a variant thereof or be a fragment thereof). In some embodiments, the PHK is from Bifidobacterium bifidum (and can have, for example, the amino acid sequence of SEQ ID NO: 65, be a variant thereof or be a fragment thereof). In some embodiments, the PHK is from Bifidobacterium gallicium (and can have, for example, the amino acid sequence of SEQ ID NO: 66, be a variant thereof or be a fragment thereof). In some embodiments, the PHK is from Bifidobacterium animalis (and can have, for example, the amino acid sequence of SEQ ID NO: 67, be a variant thereof or be a fragment thereof). In some embodiments the PHK is from Aspergillus niger (and can have, for example, the amino acid sequence of SEQ ID NO: 62, be a variant thereof or be a fragment thereof). In some embodiments, the PHK is from Aspergillus nidulans (and can have, for example, the amino acid sequence of SEQ ID NO: 71, be a variant thereof or be a fragment thereof). In some embodiments, the PHK is from Aspergillus clavatus (and can have, for example, the amino acid sequence of SEQ ID NO: 72). In some embodiments, the PHK is from Neurospora crassa (and can have, for example, the amino acid sequence of SEQ ID NO: 63, be a variant thereof or be a fragment thereof). In some embodiments, the PHK is from Lactobacillus casei (and can have, for example, the amino acid sequence of SEQ ID NO: 64, be a variant thereof or be a fragment thereof). In some embodiments, the PHK is from Lactobacillus paracasei. In some embodiment, the PHK is from Lactobacillus acidophilus (and can have, for example, the amino acid of SEQ ID NO: 69, be a variant thereof or be a fragment thereof). In some embodiments, the PHK is from Lactiplantibacillus pentosus (and can have, for example, the amino acid sequence of SEQ ID NO: 3, 5 or 68, be a variant thereof or be a fragment thereof). In some embodiments, the PHK is from Penicillium chrysogenum (and can have, for example, the amino acid sequence of SEQ ID NO: 70, be a variant thereof or be a fragment thereof). In some embodiments, the PHK is from Leuconostoc mesenteroides (and can have, for example, the amino acid sequence of SEQ ID NO: 73, be a variant thereof or be a fragment thereof). In some embodiments, the PHK is from Oenococcus oeni (and can have, for example, the amino acid sequence of SEQ ID NO: 74, be a variant thereof or be a fragment thereof).

In some embodiments, the recombinant yeast host cell comprises a further metabolic pathway (which can be engineered) to convert acetate into ethanol. This further engineered metabolic pathway can include an acetyl-coA synthase (ACS). Acetyl-coA synthases (ACS) are enzymes catalyzing the conversion of acetate into acetyl-coA and are classified in the Enzyme Commission Number class 6.2.1.1. As such, the recombinant yeast host cell can include (or be genetically engineered to include) an acetyl-coA synthase, an acetyl-coA synthase variant, an acetyl-coA synthase fragment or be encoded by a gene ortholog/paralog of the gene encoding the acetyl-coA synthase. Exemplary polypeptides having acetyl-coA synthase activity can be encoded, for example by one of the following genes Saccharomyces cerevisiae Gene ID: 850846, Arabidopsis thaliana Gene ID: 837082, Solanum lycopersicum Gene ID: 606304, Sugiyamaella lignohabitans Gene ID: 30035839 and 30034559, Triticum aestivum Gene ID: 543237, Scheffersomyces stipitis Gene ID: 4840021, Volvox carteri f. nagariensis Gene ID: 9624764, Chlamydomonas reinhardtii Gene ID: 5725731 and Candida albicans Gene ID: 3644710. In an embodiment, the polypeptide having acetyl-coA synthase activity is an ACS2 polypeptide (derived from Saccharomyces cerevisiae for example) that can have the amino acid sequence of SEQ ID NO: 56, a variant thereof, a fragment thereof or a polypeptide encoded by an ACS2 gene ortholog/paralog.

In some embodiments, the recombinant yeast host cell can also includes one or more genetic modification reducing the expression or inactivating one or more genes encoding one or more polypeptides in a pentose phosphate pathway. Alternatively, the yeast host cell can be selected based on the fact that it lacks activity in its pentose phosphate pathway. The presence of such one or more genetic modification/absence of activity in the pentose phosphate pathway favors the conversion of acetate into ethanol. This can be achieved, for example, by deleting or inactivating one or more genes encoding a polypeptide in the pentose phosphate pathway, by mutating the coding sequence of the one or more polypeptides in the pentose phosphate pathway to decrease its activity (when compared to the native polypeptide).

An exemplary polypeptide of the pentose phosphate pathway capable of functioning to convert xylose into ethanol is a transketolase (TKL). Transketolases catalyze the conversion of D-xylulose-5-phophate and aldose erythrose-4-phosphate into fructose 6-phosphate and glyceraldehyde-3-phosphate as well as the conversion of D-xylulose-5-phosphate and D-ribose-5-phosphate into sedoheptulose-7-phosphate and glyceraldehyde-3-phosphate. Transketolases are classified in the Enzyme Commission Number class 2.2.1.1. The polypeptide having transketolase activity can be native or heterologous to the recombinant yeast host cell. As such, the one or more polypeptides in the pentose phosphate pathway can be a transketolase, a transketolase variant, a transketolase fragment or be encoded by a gene ortholog/paralog of the gene encoding the transketolase.

A further exemplary polypeptide of the pentose phosphate pathway is a transaldolase (TAL), such as, for example a sedoheptulose-7-phosphate:D-glyceraldehyde-3-phosphate transaldolase. Transaldolases catalyze the conversion of sedoheptulose 7-phosphate and glyceraldehyde 3-phosphate into erythrose 4-phosphate and fructose 6-phosphate and are classified in the Enzyme Commission Number class 2.2.1.2.

A further exemplary polypeptide of the pentose phosphate pathway capable is a ribose-5-phosphate ketol-isomerase (RKI). Ribose-5-phosphate ketol-isomerases catalyze the conversion between ribose-5-phosphate and ribulose-5-phosphate and are classified in the Enzyme Commission Number class 5.3.1.6.

Yet another exemplary polypeptide of the pentose phosphate pathway is a ribulose-phosphate 3-epimerase (RPE). Ribulose-phosphate 3-epimerases catalyze the conversion of conversion between D-ribulose 5-phosphate and D-xylulose 5-phosphate and are classified in the Enzyme Commission Number class 5.1.3.1.

The yeast host cell of the present disclosure can optionally include one or more further genetic modification allowing the expression of a heterologous saccharolytic enzyme. As used in the context of the present disclosure, a “saccharolytic enzyme” can be any enzyme involved in carbohydrate digestion, metabolism and/or hydrolysis, including amylases, cellulases, hemicellulases, cellulolytic and amylolytic accessory enzymes, inulinases, levanases, and pentose sugar utilizing enzymes. amylolytic enzyme. In an embodiment, the saccharolytic enzyme is an amylolytic enzyme. As used herein, the expression “amylolytic enzyme” refers to a class of enzymes capable of hydrolyzing starch or hydrolyzed starch. Amylolytic enzymes include, but are not limited to alpha-amylases (EC 3.2.1.1, sometimes referred to fungal alpha-amylase, see below), maltogenic amylase (EC 3.2.1.133), glucoamylase (EC 3.2.1.3), glucan 1,4-alpha-maltotetraohydrolase (EC 3.2.1.60), pullulanase (EC 3.2.1.41), iso-amylase (EC 3.2.1.68) and amylomaltase (EC 2.4.1.25). In an embodiment, the one or more amylolytic enzymes can be an alpha-amylase from Aspergillus oryzae, a maltogenic alpha-amylase from Geobacillus stearothermophilus, a glucoamylase from Saccharomycopsis fibuligera, a glucan 1,4-alpha-maltotetraohydrolase from Pseudomonas saccharophila, a pullulanase from Bacillus naganoensis, a pullulanase from Bacillus acidopullulyticus, an iso-amylase from Pseudomonas amyloderamosa, and/or amylomaltase from Thermus thermophilus. Some amylolytic enzymes have been described in WO2018/167670 and are incorporated herein by reference.

For example, the yeast host cell can bear one or more genetic modifications allowing for the production of a heterologous glucoamylase. Many microbes produce an amylase to degrade extracellular starches. In addition to cleaving the last α(1-4) glycosidic linkages at the non-reducing end of amylose and amylopectin, yielding glucose, γ-amylase will cleave α(1-6) glycosidic linkages. The heterologous glucoamylase can be derived from any organism. In an embodiment, the heterologous protein is derived from a γ-amylase, such as, for example, the glucoamylase of Saccharomyces fibuligera (e.g., encoded by the glu 0111 gene). Examples of yeast host cells bearing such second genetic modifications are described in U.S. Pat. Nos. 10,385,345 and 11,332,728 both herewith incorporated in their entirety.

The yeast host cell described herein can be provided as a combination with the bacterial host cell described herein. In such combination, the yeast host cell can be provided in a distinct container from the bacterial host cell. The yeast host cell can be provided as a cell concentrate. The cell concentrate comprising the yeast host cell can be obtained, for example, by propagating the yeast host cells in a culture medium and removing at least one components of the medium comprising the propagated yeast host cell. This can be done, for example, by dehydrating, filtering (including ultra-filtrating) and/or centrifuging the medium comprising the propagated yeast host cell. In an embodiment, the yeast host cell is provided as a cream in the combination.

The yeast host cell of the present disclosure can be provided in a composition comprising starch. The composition can optionally also comprises the bacterial host cell described herein.

### Process of Using the Combination

The combination of the host cells described herein can be used to convert a biomass which comprises hexoses into ethanol. Broadly, the processes comprise contacting the yeast host cell (also referred to, in some embodiments, as a fermenting yeast) and the bacterial host cell with the biomass under conditions to allow the conversion of at least in part of the biomass into ethanol. The biomass comprises hexoses, which include but are not limited to, glucose. The biomass can comprise or be derived from starch (which can be, in some embodiments, be provided from corn).

The combination of the host cells described herein can be used to reduce the emission of greenhouse gases, such as CO₂, during the bioconversion of a biomass into alcohol. In some embodiments, the process can achieve a reduction in at least 1, 2, 3, 4, 5, 5, 6, 7, 8, 10, 11, 12, 13, 14, 15, 16, 17, 18, 19, 20, 21, 22, 23, 24, 25, 26 or 27% or higher of CO₂ when compared to a corresponding control process conducted in the absence of the combination (with a wild-type yeast for example).

The process of the present disclosure can be used to increase the fermentation during the bioconversion of a biomass into ethanol. In some embodiments, the process can achieve an increase in at least 1, 1.1, 1.2, 1.3, 1.4, 1.5, 1.6, 1.7, 1.8, 1.9, 2, 2.1, 2.2, 2.3, 2.4, 2.5, 3, 3.5, 4, 4.5, 5, 5.5, 6, 6.5, 7, 7.5, 8, 8.5, 9, 9.5, 10, 15, 20, 25, 30, 35, 40, 45, 50% or higher of ethanol yield when compared to a corresponding control process conducted in the absence of the bacterial host cell (with a fermenting yeast only for example).

Broadly, the processes comprise contacting the yeast host cell (also referred to, in some embodiments, as a fermenting yeast) and the bacterial host cell with the biomass under conditions to allow the conversion of at least in part of the biomass into ethanol. In the process of the present disclosure, biomass can first be contacted with the yeast host and then with the bacterial host cells. In such embodiment, the bacterial host cells can be contacted with the fermented biomass once a certain level of glucose has been achieved (such as, for example, once the biomass has been depleted, at least partly, from glucose). In some embodiments, the bacterial host cell is contacted with a fermentation medium having a glucose concentration equal to or less than 12.5 mM to avoid carbon catabolite repression. In some alternative embodiments, the bacterial host cell is contact with a fermentation medium having a glucose concentration higher than 12.5 mM. Alternatively, the biomass can first be contacted with the bacterial host cells and then with the yeast host cells. Also, in some embodiments, both the yeast host cells and the bacterial host cells can be contacted simultaneously with the biomass.

The biomass that can be fermented with the combination of host cells described herein includes any type of biomass known in the art and described herein. For example, the biomass can include, but is not limited to, starch, sugar and lignocellulosic materials comprising lignocellulosic fibers. Starch materials can include, but are not limited to, mashes such as corn, wheat, rye, barley, rice, or milo. The starch present in the biomass can be totally or in part in a raw form or in a gelatinized form. When the biomass comprises or is derived from corn, it can include a corn mash. Sugar materials can include, but are not limited to, sugar beets, artichoke tubers, sweet sorghum, molasses or cane. The terms “lignocellulosic material”, “lignocellulosic substrate” and “cellulosic biomass” mean any type of biomass comprising cellulose, hemicellulose, lignin, or combinations thereof, such as but not limited to woody biomass, forage grasses, herbaceous energy crops, non-woody-plant biomass, agricultural wastes and/or agricultural residues, forestry residues and/or forestry wastes, paper-production sludge and/or waste paper sludge, waste-water-treatment sludge, municipal solid waste, corn fiber from wet and dry mill corn ethanol plants and sugar-processing residues. The terms “hemicellulosics”, “hemicellulosic portions” and “hemicellulosic fractions” mean the non-lignin, non-cellulose elements of lignocellulosic material, such as but not limited to hemicellulose (i.e., comprising mannan, glucomannan and galactoglucomannan), pectins (e.g., homogalacturonans, rhamnogalacturonan I and II, and xylogalacturonan) and proteoglycans (e.g., arabinogalactan-protein). In some embodiments, the biomass can include and/or be supplemented with citric acid (especially when acetic acid or acetate is the first metabolic product).

In a non-limiting example, the lignocellulosic material can include, but is not limited to, woody biomass, such as recycled wood pulp fiber, sawdust, hardwood, softwood, and combinations thereof; grasses, such as switch grass, cord grass, rye grass, reed canary grass, miscanthus, or a combination thereof; sugar-processing residues, such as but not limited to sugar cane bagasse; agricultural wastes, such as but not limited to rice straw, rice hulls, barley straw, corn cobs, cereal straw, wheat straw, canola straw, oat straw, oat hulls, and corn fiber; stover, such as but not limited to soybean stover, corn stover; succulents, such as but not limited to, agave; and forestry wastes, such as but not limited to, recycled wood pulp fiber, sawdust, hardwood (e.g., poplar, oak, maple, birch, willow), softwood, or any combination thereof. Lignocellulosic material may comprise one species of fiber; alternatively, lignocellulosic material may comprise a mixture of fibers that originate from different lignocellulosic materials. Other lignocellulosic materials are agricultural wastes, such as cereal straws, including wheat straw, barley straw, canola straw and oat straw; corn fiber; stovers, such as corn stover and soybean stover; grasses, such as switch grass, reed canary grass, cord grass, and miscanthus; or combinations thereof.

Substrates for cellulose activity assays can be divided into two categories, soluble and insoluble, based on their solubility in water. Soluble substrates include cellodextrins or derivatives, carboxymethyl cellulose (CMC), or hydroxyethyl cellulose (HEC). Insoluble substrates include crystalline cellulose, microcrystalline cellulose (Avicel), amorphous cellulose, such as phosphoric acid swollen cellulose (PASC), dyed or fluorescent cellulose, and pretreated lignocellulosic biomass. These substrates are generally highly ordered cellulosic material and thus only sparingly soluble.

It will be appreciated that suitable lignocellulosic material may be any feedstock that contains soluble and/or insoluble cellulose, where the insoluble cellulose may be in a crystalline or non-crystalline form. In various embodiments, the lignocellulosic biomass comprises, for example, wood, corn, corn stover, sawdust, bark, molasses, sugarcane, leaves, agricultural and forestry residues, grasses such as switchgrass, ruminant digestion products, municipal wastes, paper mill effluent, newspaper, cardboard or combinations thereof.

Paper sludge is also a viable feedstock for lactate or acetate production. Paper sludge is solid residue arising from pulping and paper-making, and is typically removed from process wastewater in a primary clarifier. The cost of disposing of wet sludge is a significant incentive to convert the material for other uses, such as conversion to ethanol. Processes provided by the present invention are widely applicable. Moreover, the saccharification and/or fermentation products may be used to produce ethanol or higher value added chemicals, such as organic acids, aromatics, esters, acetone and polymer intermediates.

The fermentation process can be performed at temperatures of at least about 25° C., about 28° C., about 30° C., about 31° C., about 32° C., about 33° C., about 34° C., about 35° C., about 36° C., about 37° C., about 38° C., about 39° C., about 40° C., about 41° C., about 42° C., or about 50° C. In some embodiments, the process can be conducted at temperatures above about 30° C., about 31° C., about 32° C., about 33° C., about 34° C., about 35° C., about 36° C., about 37° C., about 38° C., about 39° C., about 40° C., about 41° C., about 42° C., or about 50° C.

In some embodiments, prior to fermentation, a step of liquefying starch can be included. The liquefaction of starch can be performed at a temperature of between about 70° C.-105° C. to allow for proper gelatinization and hydrolysis of the starch. In an embodiment, the liquefaction occurs at a temperature of at least about 70° C., 75° C., 80° C., 85° C., 90° C., 95° C., 100° C. or 105° C. Alternatively or in combination, the liquefaction occurs at a temperate of no more than about 105° C., 100° C., 95° C., 90° C., 85° C., 80° C., 75° C. or 70° C. In yet another embodiment, the liquefaction occurs at a temperature between about 80° C. and 85° C. (which can include a thermal treatment spike at 105° C.). In some embodiments, the recombinant bacterial host cell of the present disclosure is absent during the liquefaction step and is introduced to a liquefied biomass which has been cooled.

In some embodiments, the process can be used to produce ethanol at a particular rate. For example, in some embodiments, ethanol is produced at a rate of at least about 0.1 mg per hour per liter, at least about 0.25 mg per hour per liter, at least about 0.5 mg per hour per liter, at least about 0.75 mg per hour per liter, at least about 1.0 mg per hour per liter, at least about 2.0 mg per hour per liter, at least about 5.0 mg per hour per liter, at least about 10 mg per hour per liter, at least about 15 mg per hour per liter, at least about 20.0 mg per hour per liter, at least about 25 mg per hour per liter, at least about 30 mg per hour per liter, at least about 50 mg per hour per liter, at least about 100 mg per hour per liter, at least about 200 mg per hour per liter, at least about 300 mg per hour per liter, at least about 400 mg per hour per liter, at least about 500 mg per hour per liter, at least about 600 mg per hour per liter, at least about 700 mg per hour per liter, at least about 800 mg per hour per liter, at least about 900 mg per hour per liter, at least about 1 g per hour per liter, at least about 1.5 g per hour per liter, at least about 2 g per hour per liter, at least about 2.5 g per hour per liter, at least about 3 g per hour per liter, at least about 3.5 g per hour per liter, at least about 4 g per hour per liter, at least about 4.5 g per hour per liter, at least about 5 g per hour per liter, at least about 5.5 g per hour per liter, at least about 6 g per hour per liter, at least about 6.5 g per hour per liter, at least about 7 g per hour per liter, at least about 7.5 g per hour per liter, at least about 8 g per hour per liter, at least about 8.5 g per hour per liter, at least about 9 g per hour per liter, at least about 9.5 g per hour per liter, at least about 10 g per hour per liter, at least about 10.5 g per hour per liter, at least about 11 g per hour per liter, at least about 11.5 g per hour per liter, at least about 12 g per hour per liter, at least about 12.5 g per hour per liter, at least about 13 g per hour per liter, at least about 13.5 g per hour per liter, at least about 14 g per hour per liter, at least about 14.5 g per hour per liter or at least about 15 g per hour per liter.

During fermentation, the pH of the fermentation medium can be equal to or below 5.5, 5.4, 5.3, 5.2, 5.1, 5.0, 4.9, 4.8, 4.7, 4.6, 4.5, 4.4, 4.3, 4.2, 4.1, 4.0 or lower. In an embodiment, the pH of the fermentation medium (during fermentation) is between 4.0 and 5.5.

In the process described herein, it is possible to add an exogenous source (e.g., to dose) of an enzyme to facilitate saccharification or improve fermentation yield. As such, the process can comprise including one or more dose of one or more exogenous enzyme during the saccharification and/or the fermentation step. The exogenous enzyme can be provided in a purified form or in combination with other enzymes (e.g., a cocktail). In the context of the present disclosure, the term “exogenous” refers to a characteristic of the enzyme, namely that it has not been produced during the saccharification or the fermentation step, but that it was produced prior to the saccharification or the fermentation step. The exogenous enzyme that can be used during the saccharification/fermentation process can include, without limitation, an alpha-amylase, a glucoamylase, a protease, a phytase, a pullulanase, a cellulase, a xylanase, a trehalase, or any combination thereof.

In the process described herein, it is possible to add a nitrogen source (usually urea or ammonia) to facilitate saccharification or improve fermentation yield. As such, the process can comprise including one or more amount of the nitrogen source prior to or during the saccharification and/or the fermentation step.

Ethanol production can be measured using any method known in the art. For example, the quantity of ethanol in fermentation samples can be assessed using HPLC analysis. Many ethanol assay kits are commercially available that use, for example, alcohol oxidase enzyme based assays.

The processes of the present disclosure can include, in some embodiments, measuring the amount of metabolites (such as hexoses, and/or glycerol for example) present in the biomass (prior to, during and/or after the fermentation of the biomass). In some additional embodiments, the processes of the present disclosure can include distilling ethanol from the fermented biomass.

The present invention will be more readily understood by referring to the following examples which are given to illustrate the invention rather than to limit its scope.

### Example I—Biomass Comprising Acetate

TABLE 1
Description of the Lb. pentosus, Lb. plantarum and
S. cerevisiae strains used in Example I. pNH256 corresponds
to a control plasmid which does not encode and cannot express
the GLDA polypeptide of SEQ ID NO: 7. pNH256::gldA corresponds
to a plasmid encoding the GLDA polypeptide of SEQ ID NO: 7
(and thus allows the expression of the GLDA polypeptide).

| | | |
|---|---|---|
| | Parental | Genetic modifications |
| Name | cell | introduced |

Bacteria

| M17486 | This corresponds to a non-genetically modified Lb. pentosus | |
|---|---|---|
| | strain having the glycerol dehydrogenation pathway | |
| M27722 | M17486 | Cured of native plasmid DNA |
| M28318 | M27722 | ΔL-Idh1Δ |
| | | PDC having the amino acid |
| | | sequence of SEQ ID NO: 15 |
| | | ADHB having the amino acid |
| | | sequence of SEQ ID NO: 18 |
| M28635 | M28318 | ΔD-Idh1 |
| M28636 | | |
| M28637 | | |
| M29047 | M27722 | Cloning vector pNH256* |
| M17482 | This corresponds to a non-genetically modified Lb. plantarum | |
| | strain lacking the glycerol dehydrogenation pathway | |
| M29041 | M17482 | Cloning vector pNH256 |
| M29044 | M17482 | pNH256::gldA |

S. cerevisiae

| M2390 | None - this is a wild-type strain | |
|---|---|---|
| M19346 | M2390 | 1 (one) copie/genome of PHK |
| | | having the amino acid |
| | | sequence of SEQ ID NO: 1 |
| | | (encoded by the nucleic acid |
| | | sequence of SEQ ID NO: 2) |
| M20048 | M2390 | 2 (two) copies/genome of |
| | | PHK having the amino acid |
| | | sequence of SEQ ID NO: 1 |
| | | (encoded by the nucleic acid |
| | | sequence of SEQ ID NO: 2) |

It was first determined if the expression of a heterologous bi-functional phosphoketolase in Saccharomyces cerevisiae could increase acetate production. One (in S. cerevisiae strain M19346) or two copies (in S. cerevisiae strain M20048) of a nucleic acid molecule encoding the Bifidobacterium adolescensis phosphoketolase (having the amino acid sequence of SEQ ID NO: 1 and being encoded by a nucleic acid molecule having the nucleic acid sequence of SEQ ID NO: 2) was introduced per haploid genome. A corn mash fermentation was conducted with these strains and the amount of various metabolites, including acetate, was determined and compared to the parental strain M2390 (a wild-type, non-genetically modified strain). Briefly, the yeast strains were grown overnight and used to inoculated (0.05 g of drycell weight/L) a liquefied corn mash comprising 32.5% total solids (TS) and supplemented with 30 ppm of urea and 0.6 AGU/gTS of an exogenous glucoamylase. The fermentations were conducted at a temperature of 33° C. for the first 18 h and at a temperature of 31° C. for the remainder of the fermentation (18 h-52 h). As shown in Table 2, S. cerevisiae strains expressing the heterologous PHK (M19346 and M20048) were able to generate more acetate than an corresponding wild-type control (M2390).

TABLE 2
Metabolite profile (g/L) of parental S. cerevisiae
strain M2390 as well as S. cerevisiae strains
M19346 and M20048 after corn mash fermentation.

| Strain | Glucose | Glycerol | Acetate | Ethanol |
|---|---|---|---|---|
| M2309 | 0.8 | 12.3 | 0.8 | 146.64 |
| M19346 | 3.8 | 9.3 | 2.9 | 145.34 |
| M20048 | 41.9 | 8.2 | 4.7 | 122.35 |

It was then determined if various Lactiplantibacillus pentosus strains were able to co-metabolize acetate and glycerol. Different levels (2.5 to 80 mM) of acetate were added to a chemically defined medium (mCDM) spiked with 1% maltodextrin, and 150 mM glycerol. The bacterial strain was cultured at 33° C. for 72 h and the metabolites generated were characterized by HPLC. As shown in Table 3, the addition of increasing levels of acetate resulted in increased glycerol utilization and ethanol production. As expected, for every increase in mM of ethanol formed, there is approximately a 2-fold increase in mM glycerol consumed. The data presented in Table 3 suggests that the presence of acetate enables very efficient glycerol metabolism by Lb. pentosus strain M27722.

TABLE 3
Net metabolites (in mM) obtained before and after
culture of M27722 in a chemically defined medium
comprising increase amounts of acetate.

| | Added acetate (mM) | | | | | |
|---|---|---|---|---|---|---|
| Metabolite | 2.5 | 5 | 10 | 20 | 40 | 80 |
| Glycerol | −40.1 | −46.2 | −55.9 | −66.9 | −89.1 | −103.7 |
| Acetate | 2.5 | −1.0 | −19.3 | −22.0 | −24.8 | −30.7 |
| Ethanol | 12.7 | 14.8 | 19.4 | 26.8 | 35.2 | 40.8 |

Lb. pentosus strain M27722 was modified to inactivate its native lactate dehydrogenase gene L-ldh1 and allow for the expression of a Zymonas mobilis pyruvate decarboxylase and a Zymonas mobilis acetylating dehydrogenase to generate Lb. pentosus strain M28318 (Table 1). Lb. pentosus strain M28318 was further modified to inactivate its native D-ldh1 gene to generate Lb. pentosus strains M28635, M28636 and M28637 (Table 1). The strains were cultured at 33° C. in a chemically modified medium (pH=6.0) supplemented with 4.7 mM maltotriose, 150 mM glycerol and 75 mM acetate for 72 h. The amount of some of the metabolites obtained are presented in Table 4.

TABLE 4
Metabolite profile (g/L) of Lb. pentosus strains
M27722, M28318, M28635, M28636 and M28637 after 72
h of culture in a chemically defined medium medium.

| | M27722 | M28318 | M28635 | M28636 | M28637 |
|---|---|---|---|---|---|
| Lactate | 154.3 | 76.9 | 0.2 | 0.5 | 0.6 |
| Ethanol | 52. | 151.1 | 207.0 | 208.5 | 207.8 |
| Glycerol | −121.6 | −143.1 | −142.1 | −142.2 | −142.2 |
| Acetate | −43.8 | −53.2 | −49.9 | −50.1 | −50.3 |
| Maltotriose | −3.4 | −3.4 | −3.4 | −3.4 | −3.4 |
| Acetoin | −0.2 | 0.2 | 5.2 | 4.9 | 4.9 |
| Formate | 0.8 | 3.7 | 8.7 | 9.0 | 8.5 |

As is shown in Table 4, Lb. pentosus strains M28635, M28636 and M28637 produced almost no lactic acid (0.2-0.6 mM) from maltotriose. Instead, the sugar was converted almost exclusively to ethanol (>207 mM). Like the parent Lb. pentosus strains M27722 and M28318, Lb. pentosus strains M28635, M28636 and M28637 continued to efficiently utilize glycerol, but converted it to ethanol instead of lactic acid (Table 4).

A strain of Lb. plantarum (M17482) was isolated and characterized as lacking activity in the glycerol dehydrogenation pathway. It was modified with an empty plasmid (to generate Lb. plantarum M29041 (pNH256*), see Table 1) or with a plasmid encoding the GLDA polypeptide having the amino acid sequence of SEQ ID NO: 7 (to generate Lb. plantarum M29044 (pNH256*::gldA), see Table 1). The ability of Lb. plantarum strains M29041 and M29044 to utilize glycerol was compared to strain M27722 (pNH256*). The bacterial cells were cultured in the chemically defined medium (mCDM) supplemented with 4.17 mM glucose, 150 mM glycerol, 75 mM acetate and 1 mg/ml erythromycin (for plasmid vector maintenance) at 33° C. for 48 h. Metabolites concentrations were determined prior to and after the bacterial cell culture. As shown in Table 5, Lb. plantarum strain M29041 was not able to utilize glycerol. However, Lb. plantarum strain M29044, capable of expressing the GLDA polypeptide, was able to utilize glycerol just as well as Lb. plantarum strain M29047.

TABLE 5
Metabolite profile (g/L) of Lb. plantarum strains M29041, M29044 or
Lb. pentosus M29047 after 48 h of culture in a synthetic medium.

| | Net Metabolites (mM) | | | | | |
|---|---|---|---|---|---|---|
| Strain | Lactate | Glycerol | Acetate | Ethanol | Maltotriose | Citrate |
| M29041 | 1.98 | −0.34 | 0.89 | −0.04 | −1.92 | −1.70 |
| M29044 | 13.40 | −10.04 | −2.32 | 2.21 | −1.93 | −1.76 |
| M29047 | 13.29 | −9.94 | −2.30 | 2.18 | −1.93 | −1.80 |

Saccharomyces cerevisiae strain M19346 was inoculated (0.3 g of dry cell weight/L) in 30 mL of thin stillage (pH 5.0) supplemented with 1% glucose. The fermentation medium was held at a temperature of 33° C. for 24 h to consume some of the glucose. Lactiplantibacillus pentosus strain M28635 was then inoculated (1×10⁸ colony forming units) in the fermentation medium which was further supplemented with 0.5% w/v maltodextrin. The fermentation medium was further held at a temperature of 33° C. for an additional 72 h (e.g., for a total fermentation time of 96 h). The results of this fermentation, presented in Table 6, showed glycerol metabolism by the bacterium dramatically increased ethanol titer and that acetate is consumed in this process.

TABLE 6
Profiles of metabolites (in mM) obtained by fermenting thin stillage with
Saccharomyces cerevisiae strain M19346 and Lactiplantibacillus pentosus strain M28635

| | Metabolite Concentration (mM) | | | | | |
|---|---|---|---|---|---|---|
| Sampling Time | Glucose | Glycerol | Acetate | Citrate | Malate | Ethanol |
| Blank (t = 0) | 72 | 175.3 | 19.5 | 3.4 | 24.7 | 26 |
| After yeast fermentation | 0.8 | 182.6 | 23.0 | 3.5 | 29.5 | 145 |
| (t = 24 h) | | | | | | |
| After yeast-bacterial | 0.17 | 127.6 | 9.3 | 0.4 | 2.8 | 258.7 |
| co-fermentation (t = 96 h) | | | | | | |
| Change due to bacteria: | −0.63 | −55 | −13.7 | −3.1 | −26.7 | +113.7 |

### Example II—Improvement of Glycerol Utilization in the Presence of Glucose

TABLE 7
Description of the Lb. pentosus and S. cerevisiae strains used in Example
II. The GLDA/DHAKLM expression cassette refers to a single operon comprising
GDLA (having the amino acid sequence of SEQ ID NO: 7 encoded by the nucleic
acid molecule having the nucleic acid sequence of SEQ ID NO: 80), DHAK (having
the amino acid sequence of SEQ ID NO: 9 encoded by the nucleic acid molecule
having the nucleic acid sequence of SEQ ID NO: 81), DHAL (having the amino
acid sequence of SEQ ID NO: 11 encoded by the nucleic acid molecule having
the nucleic acid sequence of SEQ ID NO: 82), and DHAM (having the amino
acid sequence of SEQ ID NO: 13 encoded by the nucleic acid molecule having
the nucleic acid sequence of SEQ ID NO: 83).

| | | |
|---|---|---|
| Name | Parental cell | Genetic modifications introduced |

Lb. pentosus

| M17486 | This corresponds to a non-genetically modified Lb. pentosus | |
|---|---|---|
| | strain having the glycerol dehydrogenation pathway | |
| M27722 | M17486 | Cured of native plasmid DNA |
| M29097 | M27722 | ΔptsI |
| M29248 | M27722 | ΔglcU |
| M30124 | M27722 | ΔmanII |
| M32263 | M27722 | Δglk1 |
| M30466 | M29248 | ΔglcU |
| | | ΔmanII |
| M28316 | M27722 | ΔL-Idh1 |
| | | One copy/genome of PDC having the amino acid |
| | | sequence of SEQ ID NO: 15 encoded by the nucleic |
| | | acid sequences of SEQ ID NO: 16 |
| | | One copy/genome of ADHB having the amino acid |
| | | sequence of SEQ ID NO: 18 encoded respectively by |
| | | the nucleic acid sequences of SEQ ID NO: 19 |
| M30365 | M28316 | ΔL-Idh1 |
| | | ΔmanII |
| | | One copy/genome of PDC having the amino acid |
| | | sequence of SEQ ID NO: 15 encoded by the nucleic |
| | | acid sequences of SEQ ID NO: 16 |
| | | One copy/genome of ADHB having the amino acid |
| | | sequence of SEQ ID NO: 18 encoded respectively by |
| | | the nucleic acid sequences of SEQ ID NO: 19 |
| M30778 | M2722 | ΔL-Idh1 |
| | | ΔD-Idh1 |
| | | ΔmanII |
| | | 2 (two) copies/genome of PDC having the amino acid |
| | | sequence of SEQ ID NO: 15 encoded by the nucleic |
| | | acid sequences of SEQ ID NO: 16 and 17 |
| | | 2 (two) copies/genome of ADHB having the amino acid |
| | | sequence of SEQ ID NO: 18 encoded respectively by |
| | | the nucleic acid sequences of SEQ ID NO: 19 and 20 |
| M31817 | M30778 | ΔL-Idh1 |
| | | ΔD-Idh1 |
| | | ΔmanII |
| | | ΔglcU |
| | | 2 (two) copies/genome of PDC having the amino acid |
| | | sequence of SEQ ID NO: 15 encoded by the nucleic |
| | | acid sequences of SEQ ID NO: 16 and 17 |
| | | 2 (two) copies/genome of ADHB having the amino acid |
| | | sequence of SEQ ID NO: 18 encoded respectively by |
| | | the nucleic acid sequences of SEQ ID NO: 19 and 20 |
| M32580 | M30778 | ΔL-Idh1 |
| | | ΔD-Idh1 |
| | | ΔmanII |
| | | ΔglcU |
| | | Δglk1 |
| | | 2 (two) copies/genome of PDC having the amino acid |
| | | sequence of SEQ ID NO: 15 encoded by the nucleic |
| | | acid sequences of SEQ ID NO: 16 and 17 |
| | | 2 (two) copies/genome of ADHB having the amino acid |
| | | sequence of SEQ ID NO: 18 encoded respectively by |
| | | the nucleic acid sequences of SEQ ID NO: 19 and 20 |
| M33528 | M31817 | ΔL-Idh1 |
| | | ΔD-Idh1 |
| | | ΔmanII |
| | | ΔglcU |
| | | ΔmapTPE |
| | | 2 (two) copies/genome of PDC having the amino acid |
| | | sequence of SEQ ID NO: 15 encoded by the nucleic |
| | | acid sequences of SEQ ID NO: 16 and 17 |
| | | 2 (two) copies/genome of ADHB having the amino acid |
| | | sequence of SEQ ID NO: 18 encoded respectively by |
| | | the nucleic acid sequences of SEQ ID NO: 19 and 20 |
| M33823 | M33528 | ΔL-Idh1 |
| | | ΔD-Idh1 |
| | | ΔmanII |
| | | ΔglcU |
| | | ΔmapTPE |
| | | ΔmdxEFG |
| | | 2 (two) copies/genome of PDC having the amino acid |
| | | sequence of SEQ ID NO: 15 encoded by the nucleic |
| | | acid sequences of SEQ ID NO: 16 and 17 |
| | | 2 (two) copies/genome of ADHB having the amino acid |
| | | sequence of SEQ ID NO: 18 encoded respectively by |
| | | the nucleic acid sequences of SEQ ID NO: 19 and 20 |
| M35605 | M34439 | ΔL-Idh1 |
| | | ΔD-Idh1 |
| | | ΔmanII |
| | | ΔglcU |
| | | ΔmapTPE |
| | | ΔmdxEFG |
| | | ΔD-Idh2 |
| | | 2 (two) copies/enome of PDC having the amino acid |
| | | sequence of SEQ ID NO: 15 encoded by the nucleic |
| | | acid sequences of SEQ ID NO: 16 and 17 |
| | | 2 (two) copies/genome of ADHB having the amino acid |
| | | sequence of SEQ ID NO: 18 encoded respectively by |
| | | the nucleic acid sequences of SEQ ID NO: 19 and 20 |
| | | GLDA/DHAKLM expression cassette |
| M36538 | M35605 | ΔL-Idh1 |
| | | ΔD-Idh1 |
| | | ΔmanII |
| | | ΔglcU |
| | | ΔmapTPE |
| | | ΔmdxEFG |
| | | ΔD-Idh2 |
| | | Δrex |
| | | 2 (two) copies/genome of PDC having the amino acid |
| | | sequence of SEQ ID NO: 15 encoded by the nucleic |
| | | acid sequences of SEQ ID NO: 16 and 17 |
| | | 2 (two) copies/genome of ADHB having the amino acid |
| | | sequence of SEQ ID NO: 18 encoded respectively by |
| | | the nucleic acid sequences of SEQ ID NO: 19 and 20 |
| | | GLDA/DHAKLM expression cassette |

Culture in chemically defined medium. The base medium used for all conditions was mCDM with 150 mM glycerol and 75 mM acetate. Carbon sources provided in the base medium were glucose (50 and 12.5 mM), maltose (50 mM), maltotriose (4.17 mM), and blends of the two carbohydrates at different concentration (50 mM glucose/4.17 mM maltotriose and 12.5 mM glucose/4.17 mM maltotriose). Samples were harvested at the end point fermentation of 48 h or 72 h for HPLC analysis.

Deletion of pts in Lb. pentosus strain M29097 (del_ptsI) had no detectable effect on growth in chemically defined medium with glucose or maltotriose as the source of carbohydrate but showed a severe growth defect in media with other PTS substrates such as trehalose or cellobiose (data not shown). Metabolite analysis showed that Lb. pentosus strain M29097 strain completely lost its ability to utilize glycerol in all tested conditions (Table 9).

TABLE 9
Net metabolites (in g/L) obtained from culturing Lb. pentosus strains M27722,
and M29097 (del_ptsl) in a defined medium containing different amounts of
carbohydrates (50 mM glucose, 12.5 mM glucose, 4.17 mM maltotriose, 50 mM glucose
and 4.17 mM maltotriose, or 12.5 mM glucose and 4.17 mM maltotriose).

| | Net Metabolites (g/L) | | | | | | |
|---|---|---|---|---|---|---|---|
| Medium | Strain | Glucose | Maltotriose | Lactate | Glycerol | Acetate | Ethanol |
| 50 mM | M27722 | −9.4 | 0 | 9.2 | −1.2 | 0.4 | 0.2 |
| glucose | M29097 | −9.2 | 0 | 7.4 | −0.1 | 0.8 | 0 |
| 12.5 mM | M27722 | −2.4 | 0 | 9.7 | −7.3 | −1.5 | 1.5 |
| glucose | M29097 | −2.4 | 0 | 1.7 | −0.2 | 0.9 | −0.1 |
| 4.17 mM | M27722 | −0.1 | −1.8 | 12.3 | −10.15 | −2.4 | 2.2 |
| maltotriose | M29097 | −0.1 | −1.8 | 1.4 | −0.1 | 1.1 | −0.1 |
| 50 mM | M27722 | −9.6 | −1.6 | 11.5 | −1.5 | 0.2 | 0.2 |
| glucose + | M29097 | −9.5 | −1.8 | 9.7 | −0.1 | 0.6 | −0.1 |
| 4.17 mM | | | | | | | |
| maltotriose | | | | | | | |
| 12.5 mM | M27722 | −2.5 | −1.8 | 13.0 | −8.8 | −2.1 | 1.9 |
| glucose + | M29097 | −2.5 | −1.8 | 3.5 | 0 | 1.0 | −0.1 |
| 4.17 mM | | | | | | | |
| maltotriose | | | | | | | |

Deletion of glcU in Lb. pentosus strain M29248 (del_glcU) had virtually no effect on growth (not shown) or metabolic profile compared to the parental control (Table 10).

TABLE 10
Net metabolites (in g/L) obtained from culturing Lb. pentosus strains
M27722, and M29248 (del_glcU) in a chemically defined medium containing
different amounts of carbohydrates (50 mM glucose, or 50 mM maltose).

| | Net Metabolites (g/L) | | | | | | |
|---|---|---|---|---|---|---|---|
| Medium | Strain | Glucose | Maltose | Lactate | Glycerol | Acetate | Ethanol |
| 50 mM | M27722 | −9.4 | −0.1 | 9.4 | −1.15 | 0.3 | 0.1 |
| glucose | M29248 | −9.4 | −0.1 | 8.6 | −0.8 | 0.3 | 0 |
| 50 mM | M27722 | 0.2 | −16.5 | 17.9 | −0.9 | 0.1 | 0 |
| maltose | M29248 | 0.2 | −16.0 | 17.5 | −1.0 | 0.1 | 0 |

Deletion of EIIABCD^{mannose} in Lb. pentosus strain M30124 increased its ability to consume glycerol in the presence of glucose (Table 11). When 50 mM of glucose was present, parental strain M27722 was able to utilize less than 10 mM glycerol, while strain M30124 consumed more than 60 mM glycerol (Table 11).

TABLE 11
Net metabolites (in mM) obtained from culturing
Lb. pentosus strains M27722 and M30124 in
a defined medium containing 50 mM glucose.

| | Net Metabolites (mM) | | | | |
|---|---|---|---|---|---|
| Strain | Glucose | Lactate | Glycerol | Acetate | Ethanol |
| M27722 | −49.0 | 150.5 | −9.5 | 3.1 | 3.3 |
| M30124 | −48.2 | 236.7 | −63.2 | −25.6 | 31.0 |

When glucose was the main carbon source, the end-product metabolites from the single ΔGlcU mutant (M29248) were similar to the parent stain M27722 (Tables 10 and 12). In contrast, the single ΔMan strain showed higher glycerol consumption (Table 11 and 12). In addition, disruption of Man in ΔGlcU background (in strain M30466), further enhanced glycerol utilization compared to the single mutant ΔMan (Table 12).

TABLE 12
Net metabolites (in mM) obtained from culturing Lb. pentosus
strains M27722, M29248 (del_glcU), and M30466 (del_glcU and
del_PTSmanII) in a defined medium containing 50 mM glucose.

| | Net Metabolites (mM) | | | | |
|---|---|---|---|---|---|
| Strain | Glucose | Glycerol | Lactate | Acetate | Ethanol |
| M27722 | −49.0 | −9.5 | 150.5 | 3.1 | 3.3 |
| M29248 | −49.1 | −5.0 | 145.0 | 5.7 | 0.7 |
| M30466 | −45.9 | −92.6 | 275.1 | −41.9 | 47.0 |

Deletion of glk1 in Lb. pentosus strain M32263 improved glycerol utilization in the presence of glucose (Table 13).

TABLE 13
Net metabolites (in g/L) obtained from culturing Lb. pentosus
strains M27722, M29097 (del_ptsl), and M32263 (del_glk1)
in a defined medium containing 50 mM glucose.

| | Net Metabolites (g/L) | | | | |
|---|---|---|---|---|---|
| Strain | Glucose | Glycerol | Lactate | Acetate | Ethanol |
| M27722 | −8.2 | −1.1 | 12.7 | 0.2 | 0.1 |
| M29097 | −9.2 | −0.4 | 10.3 | 0.3 | −0.2 |
| M32263 | −5.6 | −12.6 | 23.6 | −3.2 | 2.7 |

As indicated in Table 7, some Lb. pentosus strains were designed to generate ethanol and included further genetic modifications to determine if they are able to utilize glycerol in the presence of glucose. As shown in Table 14, Lb. pentosus strains M30778, M31817, and M32580 were all able to utilize glycerol as well as generate ethanol. As shown in Table 15, Lb. pentosus strains M31817, M33528, and M33823 were also able to utilize glycerol as well as generate ethanol.

TABLE 14
Net metabolites (in g/L) obtained from culturing
Lb. pentosus strains M30778, M31817, and M32580
in a defined medium containing 50 mM glucose.

| | Net Metabolites (g/L) | | | | |
|---|---|---|---|---|---|
| Strain | Glucose | Glycerol | Lactate | Acetate | Ethanol |
| M30778 | −7.8 | −12.8 | 1.0 | −3.5 | 11.4 |
| M31817 | −7.8 | −12.8 | 1.3 | −3.0 | 11.6 |
| M32580 | −7.6 | −13.2 | 1.1 | −2.7 | 11.9 |

TABLE 15
Net metabolites (in g/L) obtained from culturing Lb. pentosus
strains M30778, M31817, M33528, and M33823 for 48 h in a chemically
defined medium spiked with 150 mM glycerol, 75 mM acetate, 25
g/L glucose, 50 g/L maltose, and 125 g/L maltodextrin..

| | Net Metabolites (g/L) | | | | | | | |
|---|---|---|---|---|---|---|---|---|
| Strain | Glucose | Maltose | Glycerol | Lactate | Acetate | Citrate | Ethanol | C4s |
| M30778 | −23.5 | −31.3 | 0.4 | 6.2 | −2.9 | −1.1 | 0.1 | 0.2 |
| M31817 | −8.8 | −41.1 | −0.5 | 7.2 | −3.0 | −1.1 | 17.9 | 3.3 |
| M33528 | −23.2 | −8.4 | −6.1 | 4.8 | −3.9 | −1.1 | 19.1 | 2.9 |
| M33823 | −24.2 | −10.3 | −10.2 | 4.1 | −4.5 | −1.1 | 18.2 | 2.4 |

It was then determined if the deletion of the maltose or the maltodextrin operon could increase glycerol utilization while maintaining ethanol production in commercial corn mashes. Commercial mashes were obtained and where either used “as is” or spiked with 150 mM glycerol and 75 mM acetate. Samples were incubated at 33° C. with shaking (150 rpm) to avoid sedimentation. As shown in Table 16, strains M33528 and M33823 improved glycerol utilization while allowing ethanol production.

TABLE 16
Net metabolites (g/L) obtained from culturing Lb. pentosus strains
M30778, M31817, M33528, and M33823 in commercial corn mashes for 48 h.

| | | | | | | | |
|---|---|---|---|---|---|---|---|
| Strain | Glucose | DP2 | DP4+ | Lactate | Glycerol | Acetate | Ethanol |

Fermentation in non-spiked commercial mash

| | | | | | | | |
|---|---|---|---|---|---|---|---|
| None (T0) | 15.7 | 30.2 | 68.1 | 0.9 | 4.4 | 0.3 | 4.2 |
| M30778 | −15.7 | −30.0 | −9.8 | 0.3 | 0.3 | 0.02 | 23.8 |
| M31817 | −10.5 | −30.1 | −0.7 | 2.8 | 0.4 | −0.2 | 20.4 |
| M33528 | −15.7 | −5.6 | −58.2 | 2.9 | −0.6 | −0.2 | 19.7 |
| M33823 | −15.2 | −6.7 | −3.6 | 2.8 | −0.8 | −0.2 | 11.8 |

Fermentation in spiked commercial mash

| | | | | | | | |
|---|---|---|---|---|---|---|---|
| None (T0) | 24.8 | 47.4 | 82.3 | 0.9 | 14.3 | 5.2 | 3.9 |
| M30778 | −21.4 | −41.2 | −2.7 | 0.3 | 0.1 | −3.6 | 26.1 |
| M31817 | −7.6 | −33.9 | +6.1 | 4.1 | −0.9 | −3.2 | 14.8 |
| M33528 | −22.2 | −2.8 | −2.7 | 5.5 | −3.6 | −3.0 | 16.4 |
| M33823 | −21.9 | −3.3 | 19.2 | 5.5 | −3.9 | −2.9 | 13.5 |

REX is a conserved transcriptional repressor that monitors the intracellular redox state of the cell by sensing NAD⁺:NADH. In many bacteria, REX represses fermentative metabolism. It was then sought to determine if the deletion of the native rex gene could further improve glycerol utilization.

Strains M32605 and M36538 were cultured, at 3300 for 24 h, in a chemically defined medium (CDM) either (i) spiked with glucose (25 g/L), glycerol 13.8 g/L) and acetate (4.4 g/L) or (ii) glucose (25 g/L), glycerol 13.8 g/L), acetate (4.4 g/L), maltose (50 g/L) and maltotriose (125 g/L).

The metabolites generated were measured using HPLC. As shown in Table 17, the deletion of the native rex gene increased both ethanol production and glycerol utilization in strain M36538 (when compared to strain M32605).

TABLE 17
Net metabolites (in g/L) obtained from culturing Lb. pentosus strains M32605
and M36538 in a chemically defined medium containing (i) spiked with glucose (25
g/L), glycerol 13.8 g/L) and acetate (4.4 g/L) or (ii) glucose (25 g/L), glycerol
13.8 g/L), acetate (4.4 g/L), maltose (50 g/L) and maltotriose (125 g/L).

| | | | | | | | | |
|---|---|---|---|---|---|---|---|---|
| | | Lactic | | Acetic | | | | |
| Strain | Glucose | acid | Glycerol | acid | Ethanol | DP4+ | DP3 | DP2 |

Fermentation in non-spiked chemically defined medium

| | | | | | | | | |
|---|---|---|---|---|---|---|---|---|
| None (T0) | 19.58 | 1.19 | 4.11 | 0.59 | 3.30 | 72.20 | 36.94 | 24.72 |
| M35605 | −17.85 | 0.71 | −2.47 | −0.21 | 13.77 | −1.27 | 0.70 | −1.82 |
| M36538 | −18.61 | 0.72 | −2.63 | −0.20 | 15.51 | −9.44 | 0.85 | −2.61 |

Fermentation in spiked chemically defined medium

| | | | | | | | | |
|---|---|---|---|---|---|---|---|---|
| None (T0) | 20.23 | 1.18 | 14.46 | 5.07 | 3.25 | 71.42 | 37.14 | 24.69 |
| M35605 | −16.55 | 0.59 | −6.89 | −3.23 | 15.03 | 0.69 | 1.02 | −0.07 |
| M36538 | −19.44 | 0.65 | −9.02 | −4.05 | 19.99 | 5.15 | 0.76 | −4.53 |

### Example III—Glycerol Utilization Lb. paracasei

TABLE 18
Description of the Lacticaseibacillus paracasei strains used in Example
III. The GLDA/DHAKLM expression cassette refers to a single operon comprising
GDLA (having the amino acid sequence of SEQ ID NO: 7 encoded by the nucleic
acid molecule having the nucleic acid sequence of SEQ ID: 87), DHAK (having
the amino acid sequence of SEQ ID NO: 84 encoded by the nucleic acid
molecule having the nucleic acid sequence of SEQ ID: 88), DHAL (having
the amino acid sequence of SEQ ID NO: 85 encoded by the nucleic acid
molecule having the nucleic acid sequence of SEQ ID: 89), and DHAM (having
the amino acid sequence of SEQ ID NO: 86 encoded by the nucleic acid
molecule having the nucleic acid sequence of SEQ ID: 90).

| Name | Parental cell | Genetic modifications introduced |
|---|---|---|
| M17005 | This corresponds to a non-genetically modified Lb. paracasei strain | |
| M21285 | M17005 | ΔL-Idh1 |
| | | PDC having the amino acid sequence of SEQ ID NO: 15 |
| | | ADHB having the amino acid sequence of SEQ ID NO: 18 |
| M22114 | M21285 | ΔL-Idh1 |
| | | PDC having the amino acid sequence of SEQ ID NO: 15 |
| | | ADHB having the amino acid sequence of SEQ ID NO: 18 |
| M34998 | M22114 | ΔL-Idh1 |
| | | ΔglcU |
| | | PDC having the amino acid sequence of SEQ ID NO: 15 |
| | | ADHB having the amino acid sequence of SEQ ID NO: 18 |
| M35937 | M34998 | ΔL-Idh1 |
| | | ΔglcU |
| | | ΔmanII |
| | | PDC having the amino acid sequence of SEQ ID NO: 15 |
| | | ADHB having the amino acid sequence of SEQ ID NO: 18 |
| | | GLDA/DHAKLM expression cassette |

It was further determined if glycerol utilization could be further improved in an Lb. paracasei host. Strains M34998 and M35937 were modified to provide a cassette for the generation of ethanol (PDC and ADHB), and to have a reduced flux in the glucose, maltose or maltodextrin catabolism via gene knock-outs (ΔglcU, ΔmanII). Strain M35937 was further modified to provide a cassette for the utilization of glycerol (GLDA and DHAKLM). Strains M34998 and M35937 were cultured, at 3300 and under shaking conditions to avoid sedimentation, in a non-spiked commercial corn mash or a commercial corn mash spinked with 2.5 g/L trehalose, 10.55 g/L glycerol, and 3.6 g/L potassium acetate for 48 h. Strain M35937 was able to utilize glycerol while producing glycerol (Table 19).

TABLE 19
Net metabolites (in g/L) obtained from culturing Lb. paracasei strains
M34998 and M35937 in a non-spiked or spiked commercial mash.

| | | | | | | | | |
|---|---|---|---|---|---|---|---|---|
| | | Lactic | | Acetic | | | | |
| Strain | Glucose | acid | Glycerol | acid | Ethanol | DP4+ | DP3 | DP2 |

Fermentation in non-spiked commercial mash

| | | | | | | | | |
|---|---|---|---|---|---|---|---|---|
| None (T0) | 14.98 | 0.20 | 4.07 | 0.25 | 2.65 | 0.00 | 28.35 | 34.87 |
| M22114 | −13.67 | 0.66 | −0.21 | 0.52 | 9.88 | 0.00 | 1.05 | −3.09 |
| M34998 | −13.78 | 0.82 | −0.25 | 0.50 | 9.87 | 0.00 | 1.68 | −3.88 |
| M35937 | −9.92 | 1.14 | −3.77 | −0.19 | 11.79 | 0.00 | 1.26 | −5.70 |

Fermentation in spiked commercial mash

| | | | | | | | | |
|---|---|---|---|---|---|---|---|---|
| None (T0) | 14.99 | 0.19 | 14.86 | 2.36 | 2.58 | 0.00 | 28.19 | 34.74 |
| M22114 | −13.81 | 0.78 | 0.10 | 0.52 | 9.54 | 0.00 | 0.84 | −4.82 |
| M34998 | −13.81 | 0.87 | 0.24 | 0.53 | 9.83 | 0.00 | 1.74 | −5.09 |
| M35937 | −10.25 | 1.19 | −8.73 | −1.81 | 15.02 | 0.00 | 1.33 | −6.42 |

While the invention has been described in connection with specific embodiments thereof, it will be understood that the scope of the claims should not be limited by the preferred embodiments set forth in the examples, but should be given the broadest interpretation consistent with the description as a whole.

### REFERENCES

Tang C T, Ruch F E Jr, Lin C C. Purification and properties of a nicotinamide adenine dinucleotide-linked dehydrogenase that serves an Escherichia coli mutant for glycerol catabolism. J Bacteriol. 1979 October; 140(1):182-7.

What is claimed is:

1. A combination for making ethanol from a biomass comprising hexoses, the combination comprising a yeast host cell and a bacterial host cell, wherein:
the bacterial host cell has:
a first metabolic pathway comprising one or more first polypeptides for converting acetate into ethanol;
a second metabolic pathway comprising one or more second polypeptides for the conversion of glycerol into dihydroxyacetone-phosphate; and
a third metabolic pathway comprising one or more third heterologous polypeptides for converting pyruvate into ethanol; and
the yeast host cell has:
a fourth metabolic pathway comprising one or more fourth polypeptides for producing glycerol; and
a fifth metabolic pathway comprising one or more fifth polypeptides for generating acetate.

2. The combination of claim 1, wherein the hexoses comprise glucose and/or the biomass comprises or is derived from corn.

3. The combination of claim 1, wherein the one or more first polypeptides comprises:
one or more native or heterologous enzymes for converting acetate into acetyl-CoA; and/or
one or more native or heterologous enzymes for converting acetyl-CoA into acetaldehyde, and optionally acetaldehyde into ethanol.

4. The combination of claim 3, wherein:
the one or more native or heterologous enzymes for converting acetate into acetyl-CoA comprise:
a polypeptide having an acetate kinase (ACK) activity;
a polypeptide having phosphotransacetylase (PTA) activity; and/or
a polypeptide having acetyl coA-synthetase (ACS) activity; and/or
the one or more native or heterologous enzymes for converting acetyl-CoA into acetaldehyde, and optionally acetaldehyde into ethanol comprise:
a polypeptide having acetaldehyde-dehydrogenase (AADH) activity;
a polypeptide having an alcohol dehydrogenase (ADH) activity); and/or
a polypeptide having a bifunctional acetaldehyde/alcohol dehydrogase (ADHE) activity.

5. The combination of claim 1, wherein the second metabolic pathway is for the dehydrogenation of glycerol.

6. The combination of claim 5, wherein the one or more second polypeptides comprise:
a native or heterologous polypeptide having glycerol dehydrogenase (GLDA) activity or a combination of the native and the heterologous polypeptides having GLDA activity,
a native or heterologous polypeptide having an ATP-dependent dihydroxyacetone kinase (DAK) activity, and/or
a native or heterologous polypeptide having a PEP-dependent dihydroxyacetone kinase (DHAKLM) activity or a combination of a native and a heterologous polypeptides having DHAKLM activity.

7. The combination of claim 1, wherein the one of or more third heterologous polypeptides comprise:
a native or heterologous polypeptide having pyruvate decarboxylase (PDC) activity, and/or
a native or heterologous polypeptide having alcohol dehydrogenase (ADH) activity.

8. The combination of claim 1, wherein the bacterial host cell is a lactic acid bacterium.

9. The combination of claim 8, wherein the bacterial host cell is from Lactiplantibacillus sp. or from Lacticaseibacillus sp.

10. The combination of claim 8, wherein the bacterial host cell has a decreased lactate dehydrogenase activity and optionally at least one inactivated native gene coding for a lactate dehydrogenase.

11. The combination of claim 1, wherein the bacterial host cell includes at least one genetic modification:
for reducing carbon catabolite repression, when compared to a control bacterial host cell lacking the at least one genetic modification; or
for decreasing the expression or inactivating at least one gene encoding a polypeptide involved in a glycolytic flux

12. The combination of claim 11, wherein:
the at least one genetic modification for reducing carbon catabolite repression comprises a genetic modification decreasing the expression or inactivating a gene encoding a phosphoenolpyruvate-dependent phosphotransferase (PTS) transporter; and/or
the at least one gene encoding the polypeptide involved in a glycolytic flux comprises a gene encoding a glucose permease, a maltose/maltodextrin transporter, a kinase and/or a transcription factor, optionally wherein the transcription factor is REX.

13. The combination of claim 1, wherein the one or more fourth polypeptides comprise:
a native or heterologous polypeptide having glycerol-3-phosphate dehydrogenase activity, and/or
a native or heterologous polypeptide having glycerol-3-phosphate phosphatase activity.

14. The combination of claim 1, the one or more fifth polypeptides for generating acetate comprise:
one or more native or heterologous polypeptides having phosphoketolase activity, wherein the phosphoketolase has single specificity or dual specificity and optionally exhibits a phosphatase activity.

15. The combination of claim 1, wherein the yeast host cell is from Saccharomyces sp.

16. A bacterial host cell for making ethanol from a biomass comprising hexoses, the bacterial host cell comprising:
a first metabolic pathway comprising one or more first polypeptides for converting acetate into ethanol;
a second metabolic pathway comprising one or more second polypeptides for the converting glycerol into dihydroxyacetone phosphate; and
a third metabolic pathway comprising one or more third heterologous polypeptides for converting pyruvate into ethanol.

17. A composition comprising the combination claim 1 and a biomass comprising hexoses.

18. A process for converting a biomass comprising hexoses into ethanol, the process comprising contacting the biomass with the combination of claim 1 under a condition to allow the conversion of at least a part of the biomass into ethanol.

19. A process for reducing the emission of CO₂ during the conversion of a biomass into ethanol, the process comprising contacting the biomass with the combination of claim 1 under a condition to allow the conversion of at least a part of the biomass into ethanol, wherein the reduction in the emission of CO₂ is observed when comparing a process performed in the absence of the bacterial host cell.

20. A process for improving the fermentation yield during the conversion of a biomass comprising hexoses into ethanol, the process comprising contacting the biomass with the combination of claim 1 under a condition to allow the conversion of at least a part of the biomass into ethanol, wherein the improvement in the fermentation yield is observed compared to a control process performed in the absence of the bacterial host cell.
